(19) United States
(12) Patent Application Publication
(10) Pub. No.: US 2026/0248971 A1
(43) Pub. Date: Aug. 27, 2026

(54) RADIOTRACER FOR POSITRON EMISSION TOMOGRAPHY (PET) IMAGING OF ALPHA1A ADRENOCEPTORS IN HUMAN BRAIN

(71) Applicants: University of Washington, Seattle, WA (US); UNITED STATES GOVERNMENT AS REPRESENTED BY THE DEPARTMENT OF VETERANS AFFAIRS, Washington, DC (US)

(72) Inventors: Eric Petrie, Seattle, WA (US); John R. Grierson, Seattle, WA (US); Kenneth T. Stout, Seattle, WA (US); Garth Terry, Seattle, WA (US)

(73) Assignees: University of Washington, Seattle, WA (US); UNITED STATES GOVERNMENT AS REPRESENTED BY THE DEPARTMENT OF VETERANS AFFAIRS, Washington, DC (US)

(21) Appl. No.: 19/382,654

(22) Filed: Nov. 7, 2025

Related U.S. Application Data
(60) Provisional application No. 63/762,486, filed on Feb. 24, 2025.

(51) Int. Cl. A61K 51/04; A61B 6/40; A61B 6/50

(52) U.S. Cl. CPC A61K 51/0459; A61B 6/4057; A61B 6/501; A61K 2123/00

(57) ABSTRACT

Radiolabeled compounds and methods of use thereof of Formula (I) or a pharmaceutically acceptable salt thereof, wherein: R¹ is a C₁-C₆ alkyl; R² is H, a C₁-C₆ alkyl, or a C₁-C₆ haloalkyl; or R¹ and R² together form a 5- or 6-membered cycloalkyl ring, a substituted or unsubstituted thiophenyl, or a substituted or unsubstituted phenyl; R³ is H, a C₁-C₆ alkyl, a C₁-C₆ haloalkyl, a C₁-C₆ hydroxyl alkyl, a C₃-C₆ cycloalkyl, a C₁-C₆ alkylnitrile, or an C₁-C₆ alkylcycloalkyl; R⁴ is alkyl or haloalkyl; R⁵ is H or halide; and L is a 2 to 10 carbon linking group; provided that: when R¹ is Me, R² is Me, R³ is H, R⁴ is —OCH₂CF₃, and L is —CH₂—CH₂—CH₂—, R⁵ is not H; and when R¹ is Me, R² is H, R³ is H, R⁴ is —OCH₂CF₃, and L is —CH₂—CH₂—CH₂—, R⁵ is not H.

### CROSS-REFERENCE TO RELATED APPLICATION

This application claims the benefit of U.S. Provisional Application No. 63/762,486, filed Feb. 24, 2025, the entire disclosure of which is hereby incorporated by reference.

### STATEMENT OF GOVERNMENT LICENSE RIGHTS

This invention was made with government support under Grant No. W81XWH20C0116, awarded by the Department of Defense. The government has certain rights in the invention.

### BACKGROUND

Excessive noradrenergic activity in central nervous system (CNS) stress-response networks is arguably the best described neurobiological contribution to the pathophysiology of posttraumatic stress disorder (PTSD). Prazosin, a drug that prevents noradrenaline from binding to α_{1A}-α_{1B}-, and α_{1D}-adrenergic receptors (aka, adrenoceptors; ARs), has been shown to robustly reduce PTSD-related nightmares and sleep disturbance in active-duty service members and in three of four trials involving combat Veterans with chronic PTSD. However, its effects in active-duty service members and Veterans are heterogeneous. In active-duty service members, the response to prazosin was found to be most robust in those with higher pretreatment systolic blood pressure. Furthermore, although prazosin is non-sedating and non-addictive, its treatment of PTSD symptoms is complicated by a short pharmacokinetic half-life, requiring administration several times per day; the possible development of orthostatic hypotension following initial doses; and significant pharmacodynamic variability, such that determining the effective dose for individual patients requires dose adjustment over time. These potential complications may explain why the majority of Veterans with PTSD receive sub-optimal doses of prazosin.

These findings indicate a need to (1) identify in advance the active-duty service members and Veterans whose PTSD symptoms will respond best to prazosin and (2) accelerate the development of PTSD medications that regulate CNS noradrenergic stress-response networks and are (a) more broadly effective, (b) active for a longer duration, (c) less likely to cause initial-dose hypotension, and (d) have a more consistent and predictable dose-effect relationship. Such efforts are hampered by a current inability to noninvasively assess CNS α₁-AR and noradrenergic stress-response network activity in living human brain.

The present disclosure addresses these and other long-felt and unmet needs in the art.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In an aspect, the present disclosure provides a method for assessment of a treatment protocol for a subject with a medical condition that is treatable by targeting an al-AR receptor, the method comprising: screening the subject for prediction of responsiveness of the subject to an α1-AR-targeted drug, wherein the screening comprises: administering an effective amount of a radiolabeled compound of Formula (I), or a pharmaceutical salt thereof, to the subject, wherein Formula (I) is

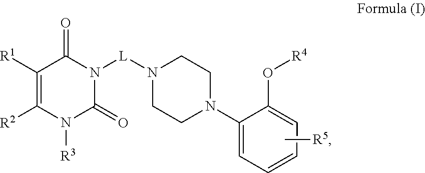

wherein: R¹ is a C₁-C₆ alkyl or a C₁-C₆ haloalkyl; R² is H, a C₁-C₆ alkyl, or a C₁-C₆ haloalkyl; or R¹ and R² together form a 5- or 6-membered cycloalkyl ring, a substituted or unsubstituted thiophenyl, or a substituted or unsubstituted phenyl; R³ is H, a C₁-C₆ alkyl, a C₁-C₆ haloalkyl, a C₁-C₆ hydroxyl alkyl, a C₃-C₆ cycloalkyl, a C₁-C₆ alkylnitrile, or an C₁-C₆ alkylcycloalkyl; R⁴ is C₁-C₆ alkyl or C₁-C₆ haloalkyl; R⁵ is H or halide; and L is a 2 to 10 carbon linking group; provided that: when R¹ is Me, R² is Me, R³ is H, R⁴ is —OCH₂CF₃, and L is —CH₂—CH₂—CH₂—, R⁵ is not H; and when R¹ is Me, R² is H, R³ is H, R⁴ is —OCH₂CF₃, and L is —CH₂—CH₂—CH₂—, R⁵ is not H; and radio-imaging at least a portion of a site of an α1-AR receptor in a subject, wherein a resultant radiograph corresponds to a predicted responsiveness of the subject to the radiolabeled compound, or a pharmaceutically acceptable salt thereof, and administering an effective amount of the α1-AR-targeted drug to the subject based on the predicted responsiveness, wherein the α1-AR-targeted drug is a non-radiolabeled derivative of the radiolabeled compound of Formula (I).

In some embodiments, R¹ is methyl or isopropyl.

In some embodiments, R² is H or Me.

In some embodiments, R² is CH₂F, CHF₂, or CF₃.

In some embodiments, R¹ and R² together form

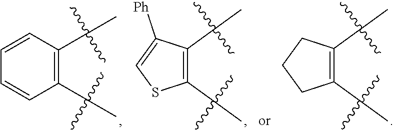 [SMILES: *c1ccccc1* ; *c1scc(-c2ccccc2)c1* ; *C1=C(*)CCC1]

In some embodiments, R³ is H, CH₂CN, methyl, ethyl, propyl, or isopropyl.

In some embodiments, R³ is fluoromethyl, fluoroethyl, or fluoropropyl.

In some embodiments, R³ is hydroxyethyl or hydroxypropyl.

In some embodiments, R³ is cyclopropyl or methyl cyclopropyl.

In some embodiments, R⁴ is Me or CH₂CF₃.

In some embodiments, R⁵ is F.

In some embodiments, L is —(CH₂)ₙ, wherein n=2 or 3.

In some embodiments, the radiolabeled compound of Formula (I) is selected from the group consisting of:

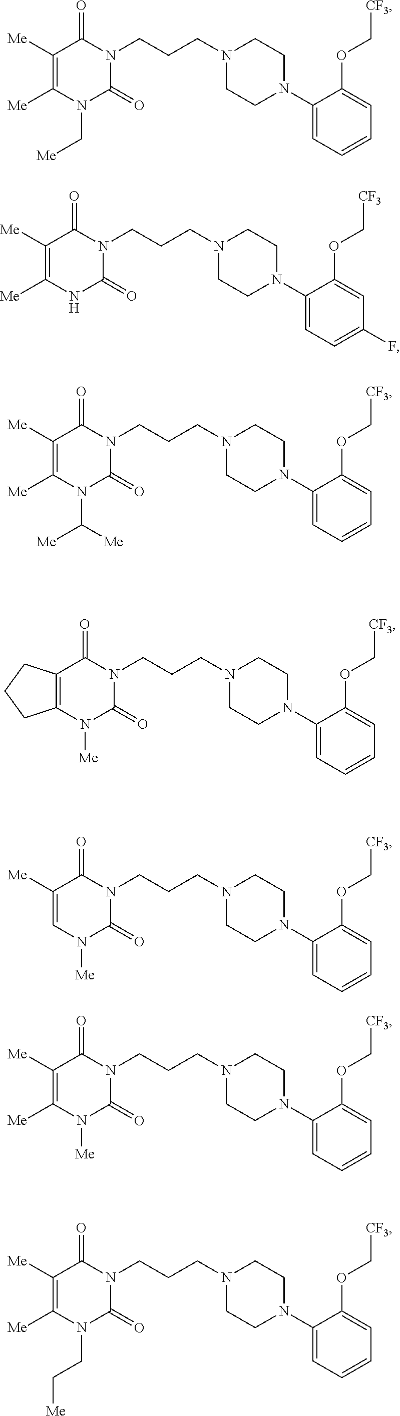

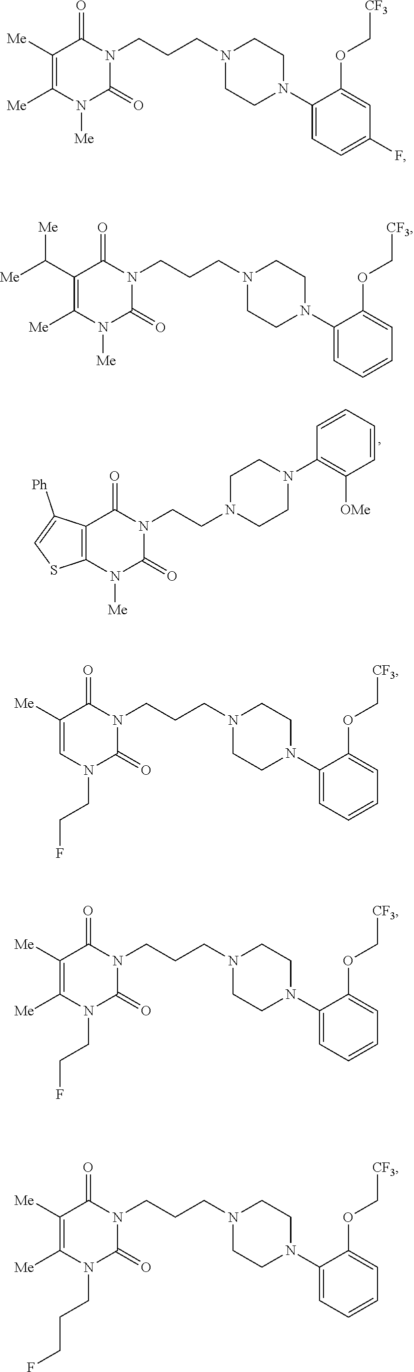

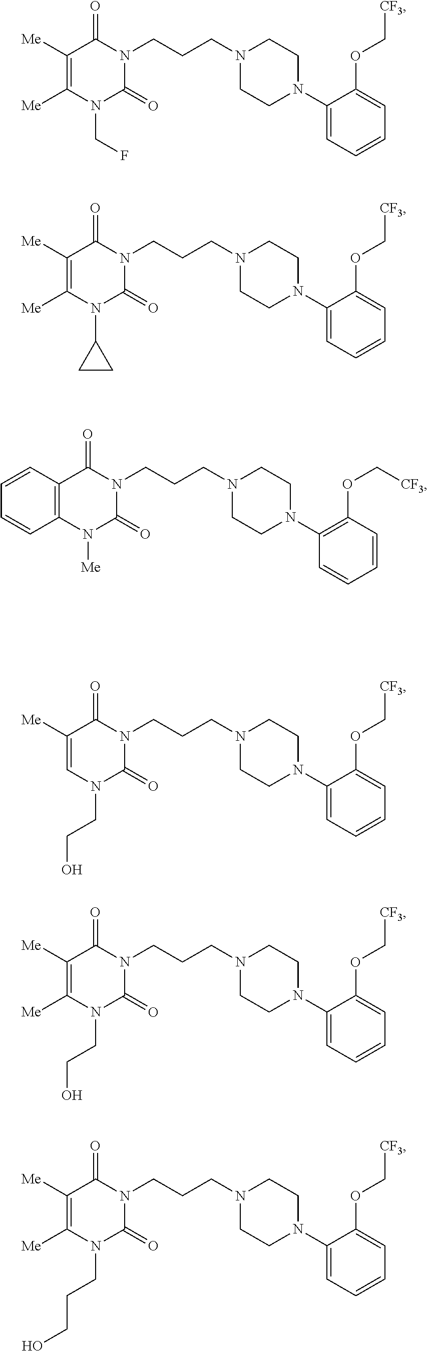 [SMILES: Cc1c(C)n(CF)c(=O)n(CCCN2CCN(c3ccccc3OCC(F)(F)F)CC2)c1=O ; Cc1c(C)n(C2CC2)c(=O)n(CCCN2CCN(c3ccccc3OCC(F)(F)F)CC2)c1=O ; Cn1c(=O)n(CCCN2CCN(c3ccccc3OCC(F)(F)F)CC2)c(=O)c2ccccc21 ; Cc1cn(CCO)c(=O)n(CCCN2CCN(c3ccccc3OCC(F)(F)F)CC2)c1=O ; Cc1c(C)n(CCO)c(=O)n(CCCN2CCN(c3ccccc3OCC(F)(F)F)CC2)c1=O ; Cc1c(C)n(CCCO)c(=O)n(CCCN2CCN(c3ccccc3OCC(F)(F)F)CC2)c1=O]

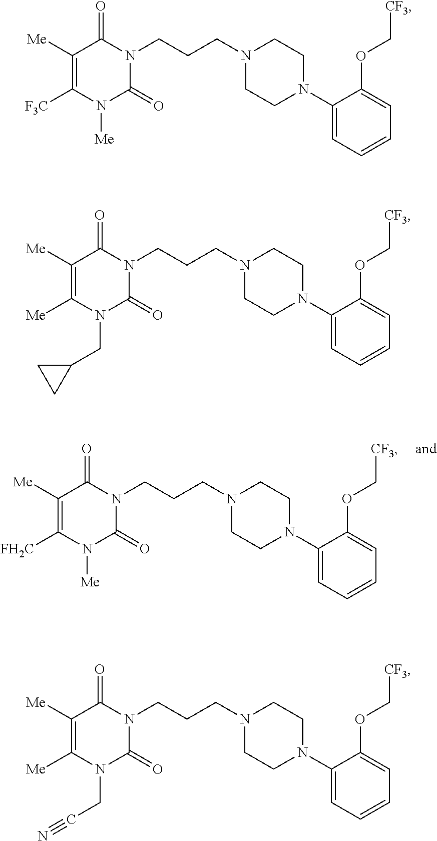

or pharmaceutically acceptable salts thereof.

In some embodiments, the radiolabeled compound comprises a carbon-11 (¹¹C), a carbon-14 (¹⁴C), a tritium (³H), or fluorine-18 (¹⁸F) radiolabel.

In some embodiments, a carbon of the alkyl, haloalkyl, hydroxyl alkyl, cycloalkyl, or alkyl-cycloalkyl group of R³ is ¹¹C.

In some embodiments, a halide of the haloalkyl group of R⁴ is ¹⁸F.

In some embodiments, a halide of the haloalkyl group of R² or a halide of the linear or branched haloalkyl group of R³ is ¹⁸F.

In some embodiments, the radio-imaging comprises positron emission tomography (PET) scanning.

In an aspect, the present disclosure provides a method for in vivo imaging of a tissue expressing an α1-AR receptor in a subject, the method comprising: administering an effective amount of a radiolabeled compound of Formula (I), or a pharmaceutical salt thereof, to the subject, wherein Formula (I) is

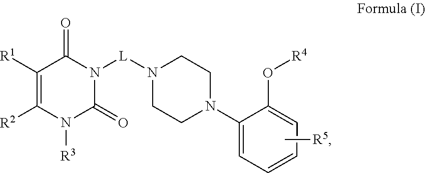

wherein: R¹ is a C₁-C₆ alkyl or a C₁-C₆ haloalkyl; R² is H, a C₁-C₆ alkyl, or a C₁-C₆ haloalkyl; or R¹ and R² together form a 5- or 6-membered cycloalkyl ring, a substituted or unsubstituted thiophenyl, or a substituted or unsubstituted phenyl; R³ is H, a C₁-C₆ alkyl, a C₁-C₆ haloalkyl, a C₁-C₆ hydroxyl alkyl, a C₃-C₆ cycloalkyl, a C₁-C₆ alkylnitrile, or an C₁-C₆ alkylcycloalkyl; R⁴ is C₁-C₆ alkyl or C₁-C₆ haloalkyl; R⁵ is H or halide; and L is a 2 to 10 carbon linking group; provided that: when R¹ is Me, R² is Me, R³ is H, R⁴ is —OCH₂CF₃, and L is —CH₂—CH₂—CH₂—, R⁵ is not H; and when R¹ is Me, R² is H, R³ is H, R⁴ is —OCH₂CF₃, and L is —CH₂—CH₂—CH₂—, R⁵ is not H; and radio-imaging at least a portion of the subject, wherein a position of a radio signal of a resultant radiograph corresponds to a site of the α1-AR receptor in the subject.

In an aspect, the present disclosure provides a radiolabeled compound of Formula (I)

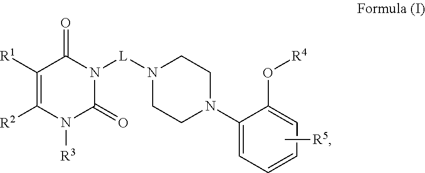

or a pharmaceutically acceptable salt thereof, wherein: R¹ is a C₁-C₆ alkyl or a C₁-C₆ haloalkyl; R² is H, a C₁-C₆ alkyl, or a C₁-C₆ haloalkyl; or R¹ and R² together form a 5- or 6-membered cycloalkyl ring, a substituted or unsubstituted thiophenyl, or a substituted or unsubstituted phenyl; R³ is H, a C₁-C₆ alkyl, a C₁-C₆ haloalkyl, a C₁-C₆ hydroxyl alkyl, a C₃-C₆ cycloalkyl, a C₁-C₆ alkylnitrile, or an C₁-C₆ alkylcycloalkyl; R⁴ is C₁-C₆ alkyl or C₁-C₆ haloalkyl; R⁵ is H or halide; and L is a 2 to 10 carbon linking group; provided that: when R¹ is Me, R² is Me, R³ is H, R⁴ is —OCH₂CF₃, and L is —CH₂—CH₂—CH₂—, R⁵ is not H; and when R¹ is Me, R² is H, R³ is H, R⁴ is —OCH₂CF₃, and L is —CH₂—CH₂—CH₂—, R⁵ is not H.

In some embodiments, R¹ is methyl or isopropyl.

In some embodiments, R² is H or Me.

In some embodiments, R² is CH₂F, CHF₂, or CF₃.

In some embodiments, R¹ and R² together form

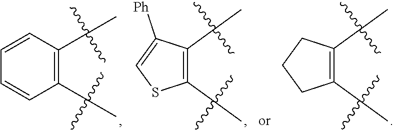 [SMILES: *c1ccccc1* ; *c1scc(-c2ccccc2)c1* ; *C1=C(*)CCC1]

In some embodiments, R³ is H, CH₂CN, methyl, ethyl, propyl, or isopropyl.

In some embodiments, R³ is fluoromethyl, fluoroethyl, or fluoropropyl.

In some embodiments, R³ is hydroxyethyl or hydroxypropyl.

In some embodiments, R³ is cyclopropyl or methyl cyclopropyl.

In some embodiments, R⁴ is Me or CH₂CF₃.

In some embodiments, R⁵ is F.

In some embodiments, L is —(CH₂)ₙ, wherein n=2 or 3.

In some embodiments, the radiolabeled compound of Formula (I) is selected from the group consisting of:

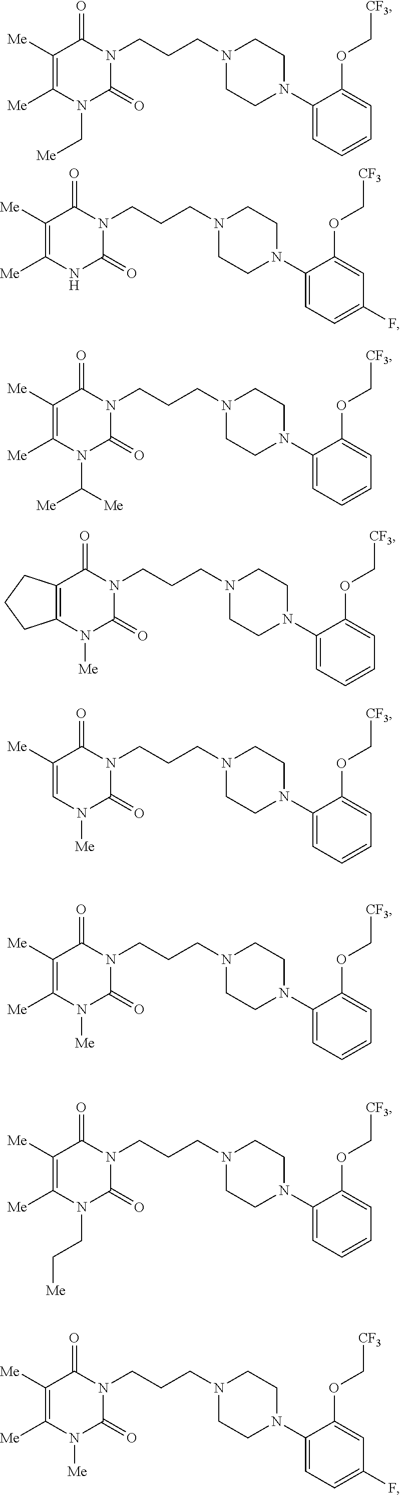

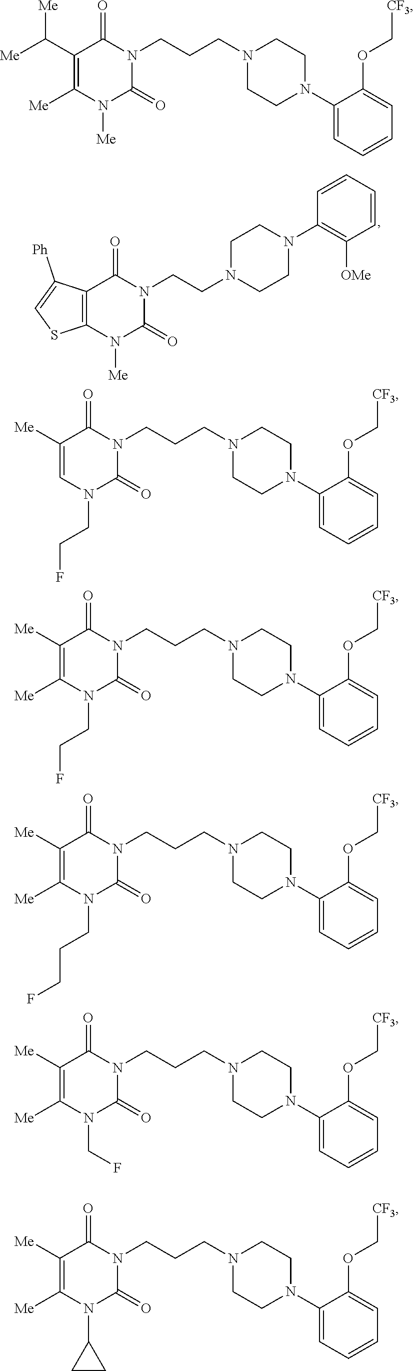

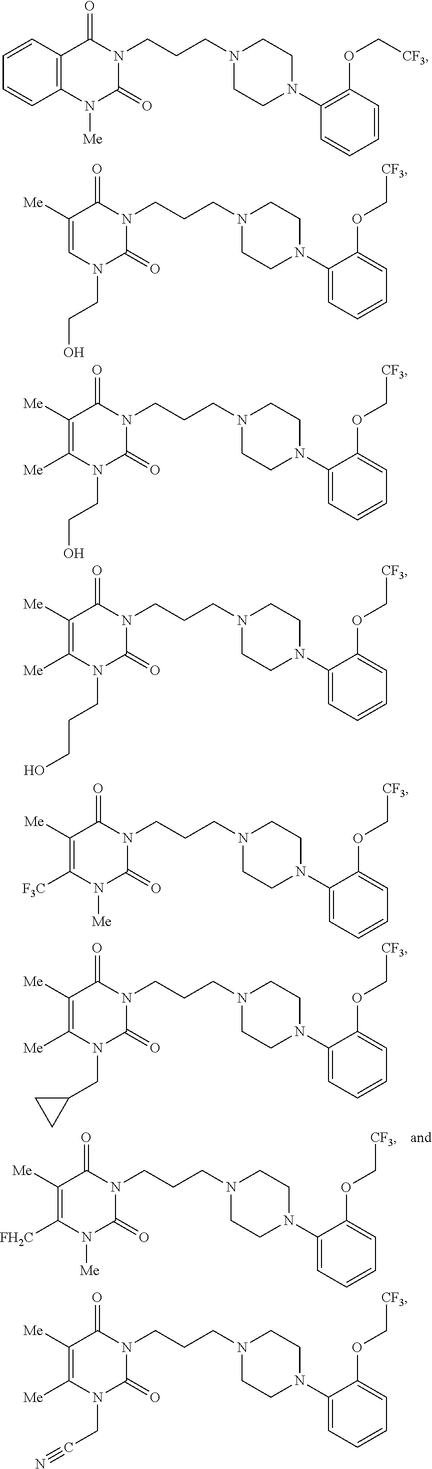

or pharmaceutically acceptable salts thereof.

In some embodiments, the radiolabeled compound comprises a carbon-11 (¹¹C), a carbon-14 (¹⁴C), a tritium (³H), or fluorine-18 (¹⁸F) radiolabel.

In some embodiments, a carbon of the alkyl, haloalkyl, hydroxyl alkyl, cycloalkyl, or alkyl-cycloalkyl group of R³ is ¹¹C.

In some embodiments, a halide of the haloalkyl group of R⁴ is ¹⁸F.

In some embodiments, a halide of the haloalkyl group of R² or a halide of the linear or branched haloalkyl group of R³ is ¹⁸F.

In an aspect, the present disclosure provides a pharmaceutical composition comprising any of the radiolabeled compounds described herein and a pharmaceutically acceptable carrier.

In an aspect, the present disclosure provides a method for synthesizing any of the radiolabeled compounds described herein, the method comprising Reaction I:

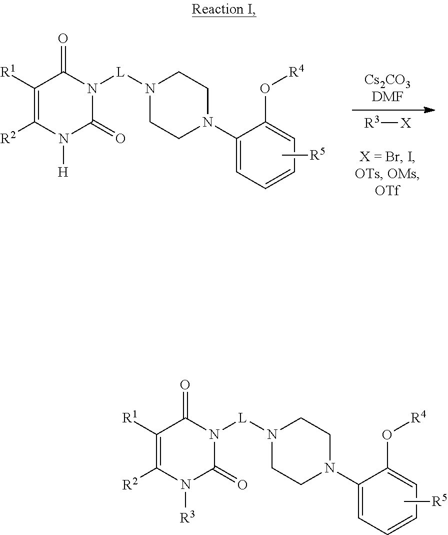

wherein R³ is a substituent selected from the group consisting of: a carbon-11 (¹¹C), carbon-14 (¹⁴C), or tritium (³H) labeled C₁-C₆ alkyl; a carbon-11 (¹¹C), carbon-14 (¹⁴C), tritium (³H), or fluorine-18 (¹⁸F) labeled C₁-C₆ haloalkyl; a carbon-11 (¹¹C), carbon-14 (¹⁴C), or tritium (³H) labeled C₁-C₆ hydroxyl alkyl; a carbon-11 (¹¹C), carbon-14 (¹⁴C), or tritium (³H) labeled C₃-C₆ cycloalkyl; a carbon-11 (¹¹C), carbon-14 (¹⁴C), or tritium (³H) labeled C₁-C₆ alkylnitrile; and a carbon-11 (¹¹C), carbon-14 (¹⁴C), or tritium (³H) labeled C₁-C₆ alkylcycloalkyl.

In an aspect, the present disclosure provides a method for predicting responsiveness of an α1-AR receptor a subject to an α1-AR-targeted drug, for assessment of a treatment protocol for a medical condition, the method comprising: administering an effective amount of any of the radiolabeled compounds of the present disclosure, or a pharmaceutically acceptable salt thereof, to the subject; and radio-imaging at least a portion of a site of an α1-AR receptor in the subject, wherein a resultant radiograph comprises a radioactivity detection, a nuclear imaging scan, or another imaging, and corresponds to a predicted positive responsiveness of the subject to the radiolabeled compound, or a pharmaceutically acceptable salt thereof.

In some embodiments, the medical condition is selected from the group consisting of: post-traumatic stress disorder (PTSD), post-traumatic headaches, behavioral dysfunction (agitation and/or aggression) in Alzheimer's disease, alcohol use disorder, urinary dysfunction, autonomic dysreflexia in patients with spinal cord injuries, congestive heart failure (CHF), and any combination thereof.

In some embodiments, the tissue is selected from the group consisting of: central nervous system tissue, cardiovascular system tissue, heart tissue, bladder tissue, prostate tissue, muscle tissue, adipose tissue, brown adipose tissue, and any combination thereof.

In some embodiments, the α1-AR-targeted drug is a α1A-AR-targeted drug, an α1B-AR-targeted drug, an α1D-AR-targeted drug, or any combination thereof.

In some embodiments, the α1-AR receptor is a α1A-AR receptor, an α1B-AR receptor, or an α1D-AR receptor.

### DESCRIPTION OF THE DRAWINGS

The foregoing aspects and many of the attendant advantages of this invention will become more readily appreciated as the same become better understood by reference to the following detailed description, when taken in conjunction with the accompanying drawings.

FIG. 1 depicts Formula (I), according to aspects of the present disclosure.

FIG. 2 depicts a reaction scheme for synthesizing a compound according to aspects of the present disclosure.

FIG. 3A depicts [C-11]ARMI in rat brain, demonstrating a radioactivity distribution consistent with that of reported and separately measured in vitro distribution of α_{1A}-AR. Time Activity Curves (TACs) reflect varying levels of radioactivity accumulation ordinal to known α_{1A}-AR distribution. Decreasing values over time suggests the radiotracer demonstrates reversible binding at the target.

FIG. 3B depicts [C-11]ARMI one hour after intraperitoneal (ip) injection of the P-glycoprotein (P-gp) efflux transporter inhibitor, tariquidar (20 mg/kg). Absence of change between baseline (FIG. 1A) and tariquidar blockade of P-gp (FIG. 1B) shows that [C-11]ARMI is not a substrate for the P-gp efflux transporter.

FIG. 3C depicts [C-11]ARMI one hour after ip injection of the subtype nonselective ai-AR antagonist, prazosin (30 mg/kg). Prazosin blocked radioactivity distribution and accumulation suggesting [C-11]ARMI is selectively binding to one or more subtypes of the ai-AR. This is also supported by the fact that prazosin reduced binding in all brain regions, reducing radioactivity to the same low level, demonstrating [C-11]ARMI has low non-specific binding (i.e., good specificity). Comparison of TACs in FIG. 3A and FIG. 3C implies the radiotracer has ~2/3 specific binding.

FIG. 3D depicts [C-11]ARMI one hour after ip injection of both tariquidar (20 mg/kg) and prazosin (30 mg/kg), further confirming the main finding of FIG. 3C by eliminating any potential confound by P-gp efflux transport of the radioligand or prazosin.

FIG. 4A depicts coronal, sagittal, and axial views (left to right) of [C-11]ARMI uptake and distribution under baseline conditions.

FIG. 4B depicts coronal, sagittal, and axial views of [C-11]ARMI uptake after inhibition of the dual P-glycoprotein (P-gp) and breast cancer resistance protein (BCRP) efflux transporters by ip injection of elacridar (7.5 mg/kg) one hour prior to radiotracer administration. Comparison of TACs (not shown) from baseline and elacridar-block scans is ~4%, reflecting an unsubstantial difference between conditions.

FIG. 5 depicts the inhibition of [³H]prazosin binding to human α_{1A}-ARs expressed in HEK cells by increasing concentrations of ARMI (triangles), compared to reference ai-AR antagonists prazosin (closed circles) and tamsulosin (open circles). Also shown is inhibition of binding of our previously disclosed 1A-AR antagonist, N-Me-RS100329 (squares). Inset shows expanded data for clarity. See Table 2 for associated Ki values, and Table 3 for selectivity screening at potential Off-target receptors. Values are means based on four (ARMI, prazosin) or three (tamsulosin) independent experiments with duplicate determinations.

FIG. 6A depicts coronal (upper left), sagittal (upper right), and axial (lower left) views of Compound 12 (with a [F-18]fluoroethyl substituent on the amine) uptake and distribution under baseline conditions.

FIG. 6B depicts TACs of Compound 12 (with a [F-18]fluoroethyl substituent on the amine) uptake and distribution under baseline conditions, which reflect varying levels of radioactivity accumulation ordinal to known α1A-AR distribution.

FIG. 6C depicts coronal (upper left), sagittal (upper right), and axial (lower left) views of Compound 12 (with a [F-18]fluoroethyl substituent on the amine) uptake and distribution under prazosin blocking conditions.

FIG. 6D depicts TACs of Compound 12 (with a [F-18]fluoroethyl substituent on the amine) uptake and distribution under prazosin blocking conditions. This demonstrates analogous concepts to that depicted in FIG. 3C. Here, the difference is 55%.

FIG. 6E depicts coronal (upper left), sagittal (upper right), and axial (lower left) views of Compound 12 (with a [F-18]fluoroethyl substituent on the amine) uptake and distribution under tariquidar blocking conditions.

FIG. 6F depicts TACs of Compound 12 (with a [F-18]fluoroethyl substituent on the amine) uptake and distribution under tariquidar blocking conditions. This demonstrates analogous concepts to FIG. 3B, and that there is no significant difference between the baseline and tariquidar.

### DETAILED DESCRIPTION

The α_{1A}-AR subtype may be involved in the pathophysiology of PTSD, and the present disclosure relates to a positron emission tomography (PET) radiotracer capable of in vivo imaging α_{1A}-ARs in human brain. Without wishing to be bound to any particular application, these PET radiotracers may help, for instance, to identify active-duty service members and Veterans whose PTSD symptoms respond well to prazosin, in addition to other patient populations. It would also allow in vivo screening of novel ax-AR-targeted drugs in human subjects, thereby expediting the development of novel medications with more desirable clinical properties, compared to prazosin, for the treatment of PTSD.

In an aspect, the present disclosure provides a radiolabeled compound of Formula (I)

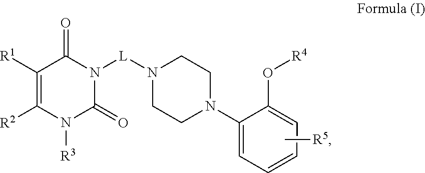

or a pharmaceutically acceptable salt thereof, wherein: R¹ is a C₁-C₆ alkyl or a C₁-C₆ haloalkyl; R² is H, a C₁-C₆ alkyl, or a C₁-C₆ haloalkyl; or R¹ and R² together form a 5- or 6-membered cycloalkyl ring, a substituted or unsubstituted thiophenyl, or a substituted or unsubstituted phenyl; R³ is H, a C₁-C₆ alkyl, a C₁-C₆ haloalkyl, a C₁-C₆ hydroxyl alkyl, a C₃-C₆ cycloalkyl, a C₁-C₆ alkylnitrile, or an C₁-C₆ alkylcycloalkyl; R⁴ is C₁-C₆ alkyl or C₁-C₆ haloalkyl; R⁵ is H or halide; and L is a 2 to 10 carbon linking group; provided that: when R¹ is Me, R² is Me, R³ is H, R⁴ is —OCH₂CF₃, and L is —CH₂—CH₂—CH₂—, R⁵ is not H; and when R¹ is Me, R² is H, R³ is H, R⁴ is —OCH₂CF₃, and L is —CH₂—CH₂—CH₂—, R⁵ is not H.

Chemical moieties referred to as univalent chemical moieties (e.g., alkyl, aryl, and the like) also encompass structurally permissible multivalent moieties, as understood by those skilled in the art. For example, while an “alkyl” moiety generally refers to a monovalent radical (e.g., CH₃CH₂—), in appropriate circumstances an “alkyl” moiety can also refer to a divalent radical (e.g., CH₂CH₂—, which is equivalent to an “alkylene” group). Similarly, under circumstances where a divalent moiety is required, those skilled in the art will understand that the term “aryl” refers to the corresponding divalent arylene group.

Terms used herein may be preceded and/or followed by a single dash, “—” or a double dash, “=”, to indicate the bond order of the bond between the named substituent and its parent moiety; a single dash indicates a single bond and a double dash indicates a double bond. In the absence of a single or double dash it is understood that a single bond is formed between the substituent and its parent moiety; further, substituents are intended to be read “left to right” unless a dash indicates otherwise. For example, C₁-C₆ alkoxycarbonyloxy and OC(O)C₁-C₆alkyl indicate the same functionality; similarly, arylalkyl and -alkylaryl indicate the same functionality.

All atoms are understood to have their normal number of valences for bond formation (e.g., 4 for carbon, 3 for N, 2 for 0, and 2, 4, or 6 for S, depending on the atom's oxidation state). On occasion a moiety can be defined, for example, as (A)ₐB, wherein a is 0 or 1. In such instances, when a is 0 the moiety is B and when a is 1 the moiety is AB.

Where a substituent can vary in the number of atoms or groups of the same kind (e.g., alkyl groups can be C₁, C₂, C₃, and the like), the number of repeated atoms or groups can be represented by a range (e.g., C₁-C₆ alkyl) which includes each and every number in the range and any and all sub ranges. For example, C₁-C₃ alkyl includes C₁, C₂, C₃, C₁₋₂, C₁₋₃, and C₂₋₃ alkyl.

The term “alkyl” as used herein, means a straight or branched chain hydrocarbon containing from 1 to 6 carbon atoms unless otherwise specified. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, and n-decyl. When an “alkyl” group is a linking group between two other moieties, then it may also be a straight or branched chain; examples include, but are not limited to, —CH₂—, —CH₂CH₂—, —CH₂CH₂CHC(CH₃)—, —CH₂CH(CH₂CH₃)CH₂—.

The term “cycloalkyl” as used herein, means a monocyclic or a bicyclic cycloalkyl ring system. Monocyclic ring systems are cyclic hydrocarbon groups containing from 3 to 8 carbon atoms, where such groups can be saturated or unsaturated, but not aromatic. In certain embodiments, cycloalkyl groups are fully saturated. Examples of monocyclic cycloalkyls include cyclopropyl, cyclobutyl, cyclopentyl, cyclopentenyl, cyclohexyl, cyclohexenyl, cycloheptyl, and cyclooctyl. Bicyclic cycloalkyl ring systems are bridged monocyclic rings or fused bicyclic rings. Bridged monocyclic rings contain a monocyclic cycloalkyl ring where two non-adjacent carbon atoms of the monocyclic ring are linked by an alkylene bridge of between one and three additional carbon atoms (i.e., a bridging group of the form —(CH₂)_{w}—, where w is 1, 2, or 3).

“Alkoxy” refers to an alkyl group, as defined herein, appended to the parent molecular moiety through an oxygen atom. Representative examples of alkoxy include, but are not limited to, methoxy, ethoxy, propoxy, 2-propoxy, butoxy, tert-butoxy, pentyloxy, and hexyloxy.

The term “thiophenyl,” as used herein, means a five-membered aromatic ring containing one sulfur atom and four carbon atoms.

“Halogen” refers to a chloro, bromo, fluoro or iodo atom radical. The term “halogen” also contemplates terms “halo” or “halide”.

The terms “haloalkyl”, “haloalkenyl” and “haloalkoxy” refer to an alkyl, alkenyl or alkoxy group, as the case may be, which is substituted with one or more halogen atoms.

The term “alkylcycloalkyl” as used herein, means a (CH₂)ₙ-cycloalkyl group, where n=1-6.

The term “alkylnitrile” as used herein, means a (CH₂)ₙ— CN group, where n=1-6.

The term “substituted” means that an atom or group of atoms formally replaces hydrogen as a “substituent” attached to another group. The term “substituted”, unless otherwise indicated, refers to any level of substitution, e.g., mono-, di-, tri-, tetra-, penta-, or higher substitution, where such substitution is permitted (e.g., results in a stable compound). The substituents are independently selected, and substitution may be at any chemically accessible position. It is to be understood that substitution at a given atom is limited by valency. The phrase “optionally substituted” means substituted or unsubstituted. The term “substituted” means that at least hydrogen atom is replaced with a substituent. A single divalent substituent, e.g., oxo, can replace two hydrogen atoms.

The compound or a pharmaceutically acceptable salt thereof of the present invention may be labeled with isotopes of hydrogen, for example, ²H. The deuterated isotopolog compound can be useful to help improve metabolic stability or determine or measure the effectiveness of the compound of the present invention, for example, by characterizing the site of action or mode of action, or binding affinity to a pharmacologically important site of action. Such ²H isotopically labeled compounds can generally be prepared by conventional techniques known to those skilled in the art.

As used herein, “stereoisomer” refers to compounds that differ in the chirality of one or more stereocenters. Stereoisomers include enantiomers and diastereomers.

As used herein, the term “pharmaceutically acceptable salt” refers to a salt that can be used as a medicament. When the compound or polyfunctional molecule disclosed herein has an acidic group, it can be reacted with a base to form a basic salt (also referred to as a “base addition salt”), and when it has a basic group, it can be reacted with an acid to form an acidic salt (also referred to as an “acid addition salt”). Examples of a “basic salts” include alkali metal salts such as sodium, potassium, and lithium salts; alkaline earth metal salts such as magnesium and calcium salts; organic base salts such as N-methylmorpholine, triethylamine, tributylamine, diisopropylethylamine, dicyclohexylamine, N-methylpiperidine, pyridine, 4-pyrrolidinopyridine, and picoline salts; and amino acid salts such as glycine, lysine, arginine, ornithine, glutamic acid, and aspartic acid salts. Examples of the “acidic salts” include inorganic acid salts such as hydrohalide (e.g., hydrofluoride, hydrochloride, hydrobromide, hydroiodide), nitrate, perchlorate, sulfate, and phosphate salts; lower alkanesulfonate salts (e.g., methanesulfonate, trifluoromethanesulfonate, ethanesulfonate), arylsulfonate (e.g., benzenesulfonate, p-toluenesulfonate salts), organic acid salts such as acetate, malate, fumarate, succinate, citrate, ascorbate, tartrate, oxalate, and maleate salts; and amino acid salts such as glycinate, lysinate, argininate, ornithinate, glutamate, and aspartate salts.

As used herein, the term “treat” refers to medical management of a disease, disorder, or condition of a subject (e.g., a human). Treatment can encompass any indicia of success in the treatment or amelioration of a disease or condition, including any parameter such as abatement, remission, diminishing of symptoms or making the disease or condition more tolerable to the subject, slowing in the rate of degeneration or decline, and/or making the degeneration less debilitating. The treatment or amelioration of symptoms can be based on objective or subjective parameters, including the results of an examination by a physician. Accordingly, the term “treating” includes the administration of the compounds and/or compositions of the present disclosure to alleviate, or to arrest or inhibit development of the symptoms or conditions associated with disease or condition. The term “therapeutically effective” refers to an amount of the compound or composition that results in a therapeutic effect and can be readily determined.

The compounds disclosed herein can be administered with one or more pharmaceutically acceptable carriers. Any suitable pharmaceutically acceptable carrier can be used with the compounds of the disclosure. The term “pharmaceutically acceptable” as used herein in reference to a material (e.g., carrier, excipient, or diluent) refers to a material that is compatible for use in a human subject. Such includes physiologically acceptable solutions or vehicles that are harmless or do not cause any significant specific or non-specific immune reaction to an organism or do not abrogate the biological activity of the active compound. For formulation of the composition into a liquid preparation, saline, sterile water, Ringer's solution, buffered physiological saline, albumin infusion solution, dextrose solution, maltodextrin solution, glycerol, ethanol, and mixtures thereof may be used as a pharmaceutically acceptable excipient or carrier. If necessary, other conventional additives such as thickeners, diluents, buffers, preservatives, surface active agents, antioxidants, and bacteriostatic agents may be added. Further, diluents, dispersants, surfactants, binders, and lubricants may be additionally added to the composition to prepare injectable formulations such as aqueous solutions, suspensions, and emulsions, oral formulations such as pills, capsules, granules, or tablets, or powdered formulations, and aerosolized formulations such as liquids or powders.

A thorough discussion of pharmaceutically acceptable excipients and salts is available in Remington's Pharmaceutical Sciences, 18th Edition (Easton, Pa.: Mack Publishing Company, 1990).

For example, sterile saline and phosphate-buffered saline at physiological pH can be used. Preservatives, stabilizers, dyes and even flavoring agents can be provided in the pharmaceutical composition. For example, sodium benzoate, sorbic acid and esters of p-hydroxybenzoic acid can be added as preservatives. Id. at 1449. In addition, antioxidants and suspending agents can be used. Id.

Additionally, auxiliary substances, such as wetting or emulsifying agents, biological buffering substances, surfactants, and the like, can be present in such vehicles. A biological buffer can be any solution which is pharmacologically acceptable, and which provides the formulation with the desired pH, i.e., a pH in the physiologically acceptable range. Examples of buffer solutions include saline, phosphate buffered saline, Tris buffered saline, Hank's buffered saline and the like.

Depending on the intended mode of administration, the pharmaceutical compositions can be in the form of solid, semi-solid or liquid dosage forms, such as, for example, tablets, suppositories, pills, capsules, powders, liquids, suspensions, creams, ointments, lotions or the like, preferably in unit dosage form suitable for single administration of a precise dosage. The compositions will include an effective amount of the selected drug in combination with a pharmaceutically acceptable carrier and, in addition, can include other pharmaceutical agents, adjuvants, diluents, buffers, and the like.

The disclosure includes a pharmaceutical composition comprising a compound of the disclosure including isomers, racemic or non-racemic mixtures of isomers, or pharmaceutically acceptable salts or solvates thereof together with one or more pharmaceutically acceptable carriers, and optionally other therapeutic and/or prophylactic ingredients.

In general, the compounds of the disclosure will be administered in a therapeutically effective amount by any of the accepted modes of administration. Suitable dosage ranges depend upon numerous factors such as the severity of the disease to be treated, the age and relative health of the subject, the potency of the compound used, the route and form of administration, the indication towards which the administration is directed, and the preferences and experience of the medical practitioner involved. One of ordinary skill in the art of treating such diseases will be able, without undue experimentation and in reliance upon personal knowledge and the disclosure of this application, to ascertain a therapeutically effective amount of the compounds of the disclosure for a given disease.

Thus, the compounds of the disclosure can be administered as pharmaceutical formulations including those suitable for nasal or parenteral (including intramuscular, intra-arterial, intrathecal, subcutaneous and intravenous) administration or in a form suitable for administration by inhalation or insufflation. The preferred manner of administration is intravenous.

For solid compositions, conventional nontoxic solid carriers include, for example, pharmaceutical grades of mannitol, lactose, starch, magnesium stearate, sodium saccharin, talc, cellulose, glucose, sucrose, magnesium carbonate, and the like. Liquid pharmaceutically administrable compositions can, for example, be prepared by dissolving, dispersing, and the like, an active compound as described herein and optional pharmaceutical adjuvants in an excipient, such as, for example, water, saline, aqueous dextrose, glycerol, ethanol, and the like, to thereby form a solution or suspension. If desired, the pharmaceutical composition to be administered can also contain minor amounts of nontoxic auxiliary substances such as wetting or emulsifying agents, pH buffering agents and the like, for example, sodium acetate, sorbitan monolaurate, triethanolamine sodium acetate, triethanolamine oleate, and the like. Actual methods of preparing such dosage forms are known, or will be apparent, to those skilled in this art for example, see Remington's Pharmaceutical Sciences, referenced above.

Parenteral formulations can be prepared in conventional forms, either as liquid solutions or suspensions, solid forms sinkable for solubilization or suspension in liquid prior to injection, or as emulsions. Preferably, sterile injectable suspensions are formulated according to techniques known in the art using suitable carriers, dispersing or wetting agents and suspending agents. The sterile injectable formulation can also be a sterile injectable solution or a suspension in a nontoxic parenterally acceptable diluent or solvent. Among the acceptable vehicles and solvents that can be employed are water, Ringer's solution and isotonic sodium chloride solution. In addition, sterile, fixed oils, fatty esters or polyols are conventionally employed as solvents or suspending media. In addition, parenteral administration can involve the use of a slow release or sustained release system such that a constant level of dosage is maintained.

Parenteral administration includes intraarticular, intravenous, intramuscular, intradermal, intraperitoneal, and subcutaneous routes, and include aqueous and nonaqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. Administration via certain parenteral routes can involve introducing the formulations of the disclosure into the body of a patient through a needle or a catheter, propelled by a sterile syringe or some other mechanical device such as a continuous infusion system. A formulation provided by the disclosure can be administered liming a syringe, injector, pump, or any other device recognized in the art for parenteral administration.

Preferably, sterile injectable suspensions are formulated according to techniques known in the art using suitable carriers, dispersing or wetting agents and suspending agents. The sterile injectable formulation can also be a sterile injectable solution or a suspension in a nontoxic parenterally acceptable diluent or solvent. Among the acceptable vehicles and solvents that can be employed are water, Ringer's solution and isotonic sodium chloride solution. In addition, suede, fixed oils, fatty esters or polyols are conventionally employed as solvents or suspending media. In addition, parenteral administration can involve the use of a slow release or sustained release system such that a constant level of dosage is maintained.

Preparations according to the disclosure for parenteral administration include sterile aqueous or non-aqueous solutions, suspensions, or emulsions. Examples of non-aqueous solvents or vehicles are propylene glycol, polyethylene glycol, vegetable oils, such as olive oil and corn oil, gelatin, and injectable organic esters such as ethyl oleate. Such dosage forms can also contain adjuvants such as preserving, wetting, emulsifying, and dispersing agents. They can be sterilized by, for example, filtration through a bacteria-retaining filter, by incorporating sterilizing agents into the composition, by irradiating the compositions, or by heating the compositions. They can also be manufactured using sterile water, or some other sterile injectable medium, immediately before use.

The formulations can optionally contain an isotonicity agent. The formulations preferably contain an isotonicity agent and glycerin is the most preferred isotonicity agent. The concentration of glycerin, when it is used, is in the range known in the art, such as for example, about 1 mg/mL to about 20 mg/mL. The pH of the parenteral formulations can be controlled by a buffering agent such as phosphate, acetate, TRIS or L-arginine. The concentration of the buffering agent is preferably adequate to provide buffering of the pH during storage to maintain the pH at a target pH±0.2 pH unit. The preferred pH is between about 7 and about 8 when measured at room temperature.

Other additives, such as a pharmaceutically acceptable solubilizers like Tween 20® (polyoxyethylene (20) sorbitan monolaurate), Tween 40® (polyoxyethylene (20) sorbitan monopalmitate), Tween 80® (polyoxyethylene (20) sorbitan monooleate), Pluronic F68® (polyoxyethylene polyoxypropylene block copolymers), and PEG (polyethylene glycol) can optionally be added to the formulation, and can be useful if the formulations will contact plastic materials. In addition, the parenteral formulations can contain various antibacterial and antifungal agents, for example, parabens, chlorobutanol, phenol, sorbic acid, thimerosal, and the like.

Sterile injectable solutions are prepared by incorporating one or more of the compounds of the disclosure in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. Thus, for example, a parenteral composition suitable for administration by injection is prepared by stirring 1.5% by weight of active ingredient in 10% by volume propylene glycol and water. The solution is made isotonic with sodium chloride and sterilized.

The pharmaceutical compositions of the disclosure can also be administered by nasal aerosol or inhalation. Such compositions are prepared according to techniques well known in the art of pharmaceutical formulation and can be prepared as solutions in saline, employing benzyl alcohol or other suitable preservatives, absorption promoters to enhance bioavailability, propellants such as fluorocarbons or nitrogen, and/or other conventional solubilizing or dispersing agents.

The compounds of the disclosure can be formulated for aerosol administration, particularly to the respiratory tract and including intranasal administration. The compound will generally have a small particle size for example of the order of 5 microns or less. Such a particle size can be obtained by means known in the art, for example by micronization. The active ingredient is provided in a pressurized pack with a suitable propellant such as a chlorofluorocarbon (CFC) for example dichlorodifluoromethane, trichlorofluoromethane, or dichlorotetrafluoroethane, carbon dioxide or other suitable gas. The aerosol can conveniently also contain a surfactant such as lecithin. The dosing can be controlled by a metered valve. Alternatively, the active ingredients can be provided in a form of a dry powder, for example a powder mix of the compound in a suitable powder base such as lactose, starch, starch derivatives such as hydroxypropylmethyl cellulose and polyvinylpyrrolidine (PVP). The powder carrier will form a gel in the nasal cavity. The powder composition can be presented in unit dose form, for example, in capsules or cartridges of e.g., gelatin or blister packs from which the powder can be administered by means of an inhaler.

A pharmaceutically or therapeutically effective amount of the composition will be delivered to the subject. The precise effective amount will vary from subject to subject and will depend upon the species, age, the subject's size and health, the nature and extent of the condition being treated, recommendations of the treating physician, and the therapeutics or combination of therapeutics selected for administration. Thus, the effective amount for a given situation can be determined by routine experimentation. For purposes of the disclosure, generally a therapeutic amount will be in the range of about 0.01 mg/kg to about 250 mg/kg body weight, more preferably about 0.1 mg/kg to about 10 mg/kg, in at least one dose. In larger mammals the indicated daily dosage can be from about 1 mg to 300 mg, one or more times per day, more preferably in the range of about 10 mg to 200 mg. The subject can be administered as many doses as is required to reduce and/or alleviate the signs, symptoms, or causes of the disorder in question, or bring about any other desired alteration of a biological system. When desired, formulations can be prepared with enteric coatings adapted for sustained or controlled release administration of the active ingredient.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself or it can be the appropriate number of any of these in packaged form.

The individual can be any animal, such as a mammal, bird, reptile, or fish. Exemplary mammalian categories include rodents, primates, canines, felines, ungulates, lagomorphs, and the like. For example, the individual can be a human, monkey, ape or other primate, mouse, rat or other rodent, dog, cat, pig, horse, cow, or rabbit, etc.

As used herein, the term “treatment” means providing an ameliorative, curative, or preventative effect on the disorder or condition. In some embodiments, treatment includes preventing the escalation or progression, or slowing the rate of escalation or progression, of the condition (as compared to no or other treatment). In the context of depression (more described below), treatment includes reversing or reducing neuroinflammation, reversing or reducing oxidative stress, promoting or increasing neurite outgrowth, and promoting neurogenesis. In an embodiment, treatment of depression includes decreasing anhedonia, decreasing despair, improving self-care, and improving motivational behavior.

In some embodiments, R¹ is methyl or isopropyl.

In some embodiments, R² is H or Me.

In some embodiments, R² is CH₂F, CHF₂, or CF₃.

In some embodiments, when R¹ and R² together form a 5- or 6-membered cycloalkyl ring, the cycloalkyl ring is unsubstituted. In some embodiments, when R¹ and R² together form a thiophenyl, the thiophenyl is substituted with a phenyl group. In some embodiments, when R¹ and R² together form a phenyl, the phenyl is unsubstituted.

In some embodiments, R¹ and R² together form

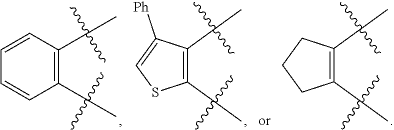 [SMILES: *c1ccccc1* ; *c1scc(-c2ccccc2)c1* ; *C1=C(*)CCC1]

In some embodiments, R³ is a C₁-C₆ alkyl, a C₁-C₆ haloalkyl, a C₁-C₆ hydroxyl alkyl, a C₃-C₆ cycloalkyl, a C₁-C₆ alkylnitrile, or an C₁-C₆ alkylcycloalkyl.

In some embodiments, R³ is H, CH₂CN, methyl, ethyl, propyl, or isopropyl.

In some embodiments, R³ is CH₂CN, methyl, ethyl, propyl, or isopropyl.

In some embodiments, R³ is fluoromethyl, fluoroethyl, or fluoropropyl.

In some embodiments, R³ is hydroxyethyl or hydroxypropyl.

In some embodiments, R³ is cyclopropyl or methyl cyclopropyl.

In some embodiments, R⁴ is Me or CH₂CF₃.

In some embodiments, R⁵ is F.

In some embodiments, the radiolabeled compound is selected from the group consisting of:

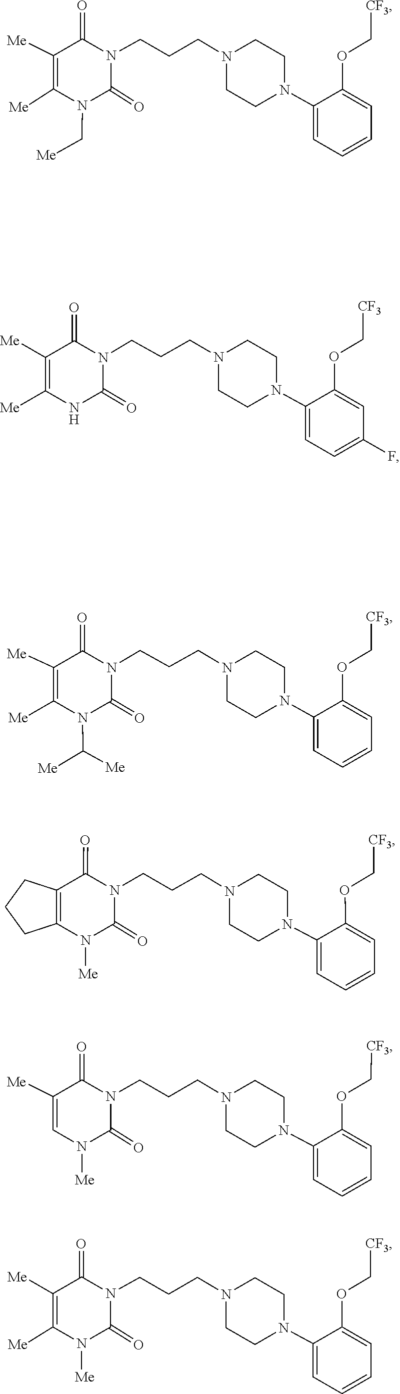 [SMILES: CCn1c(C)c(C)c(=O)n(CCCN2CCN(c3ccccc3OCC(F)(F)F)CC2)c1=O ; Cc1[nH]c(=O)n(CCCN2CCN(c3ccc(F)cc3OCC(F)(F)F)CC2)c(=O)c1C ; Cc1c(C)n(C(C)C)c(=O)n(CCCN2CCN(c3ccccc3OCC(F)(F)F)CC2)c1=O ; Cn1c2c(c(=O)n(CCCN3CCN(c4ccccc4OCC(F)(F)F)CC3)c1=O)CCC2 ; Cc1cn(C)c(=O)n(CCCN2CCN(c3ccccc3OCC(F)(F)F)CC2)c1=O ; Cc1c(C)n(C)c(=O)n(CCCN2CCN(c3ccccc3OCC(F)(F)F)CC2)c1=O]

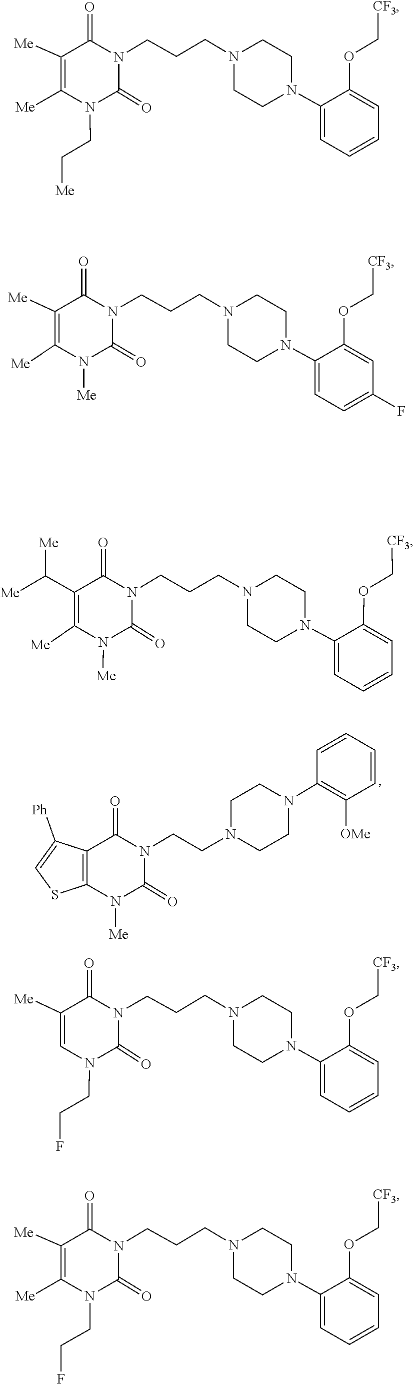

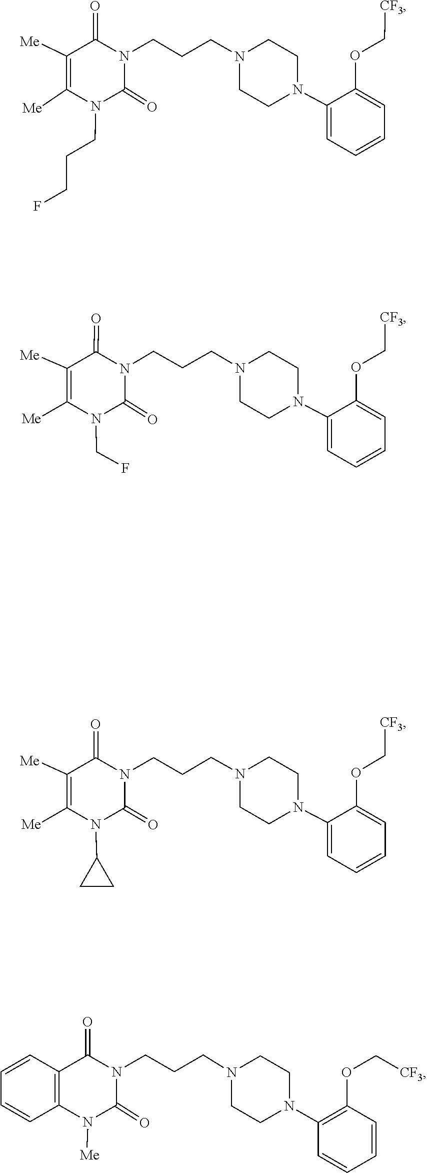 [SMILES: Cc1c(C)n(CCCF)c(=O)n(CCCN2CCN(c3ccccc3OCC(F)(F)F)CC2)c1=O ; Cc1c(C)n(CF)c(=O)n(CCCN2CCN(c3ccccc3OCC(F)(F)F)CC2)c1=O ; Cc1c(C)n(C2CC2)c(=O)n(CCCN2CCN(c3ccccc3OCC(F)(F)F)CC2)c1=O ; Cn1c(=O)n(CCCN2CCN(c3ccccc3OCC(F)(F)F)CC2)c(=O)c2ccccc21]

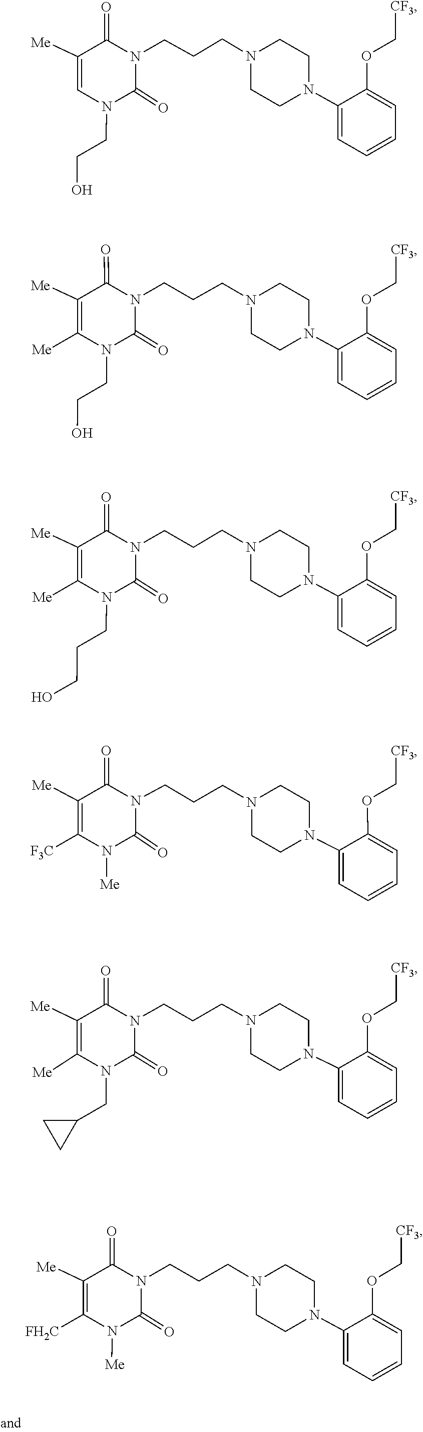

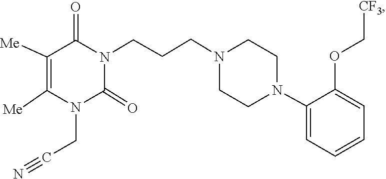 [SMILES: Cc1c(C)n(CC#N)c(=O)n(CCCN2CCN(c3ccccc3OCC(F)(F)F)CC2)c1=O]

or pharmaceutically acceptable salts thereof.

In some embodiments, the radiolabeled compound is 6-methyl-N-methyl-RS100329 (“ARMI”),

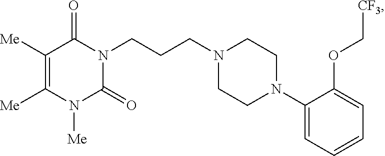 [SMILES: Cc1c(C)n(C)c(=O)n(CCCN2CCN(c3ccccc3OCC(F)(F)F)CC2)c1=O]

or a pharmaceutically acceptable salt thereof.

As used herein, “radiolabeled compound of Formula (I)” refers to a compound according to Formula (I) wherein one or more atoms defining one of the substituents R¹, R², R³, R⁴, or R⁵ has been substituted with a radioactive isotope.

In some embodiments, the radiolabeled compound comprises a carbon-11 (¹¹C), a carbon-14 (¹⁴C), a tritium (³H), or fluorine-18 (¹⁸F) radiolabeled compound. The radiolabeled compound of Formula (I) may be radiolabeled, for instance, at any one of the R¹ position, the R² position, the R³ position, the R⁴ position, the R⁵ position, or combinations thereof. In some embodiments, the radiolabeled compound is radiolabeled at the R³ position. In some embodiments, the radiolabeled compound is radiolabeled at the R⁴ position. For instance, without limitation, Compound 6 may be radiolabeled with a carbon-11 at the R³ position methyl and/or a fluorine-18 at the R⁴ position, whereas Compound 11 may be radiolabeled with either one or more carbon-11 and/or fluorine-18 atoms at the R³ position ethyl fluoride and/or a fluorine 18 at the R⁴ position. The above examples should be understood as illustrative and not limiting, and that additional radiolabeling may be done at other positions for the above example compounds.

In some embodiments, a carbon of the alkyl, haloalkyl, hydroxyl alkyl, cycloalkyl, or alkyl-cycloalkyl group of R³ of the radiolabeled compound is ¹¹C.

In some embodiments, a halide of the haloalkyl group of R⁴ of the radiolabeled compound is ¹⁸F.

In some embodiments, a halide of the haloalkyl group of R² or a halide of the linear or branched haloalkyl group of R³ of the radiolabeled compound is ¹⁸F.

In some aspects, the present disclosure relates to a pharmaceutical composition comprising any of the radiolabeled compounds of Formula (I) described herein and a pharmaceutically acceptable carrier.

### Synthesis and Characterization of 6-Methyl-N-Methyl-Rs100329 HCL (“ARMI”)

Example compound ARMI has the below structure:

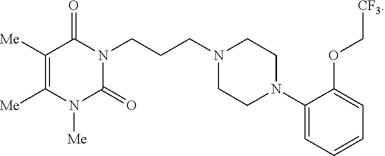 [SMILES: Cc1c(C)n(C)c(=O)n(CCCN2CCN(c3ccccc3OCC(F)(F)F)CC2)c1=O]

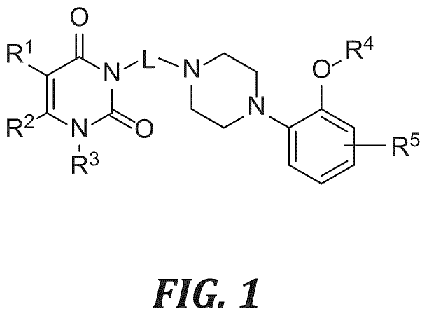

In this regard, ARMI is an example of the general structure described in FIG. 1.

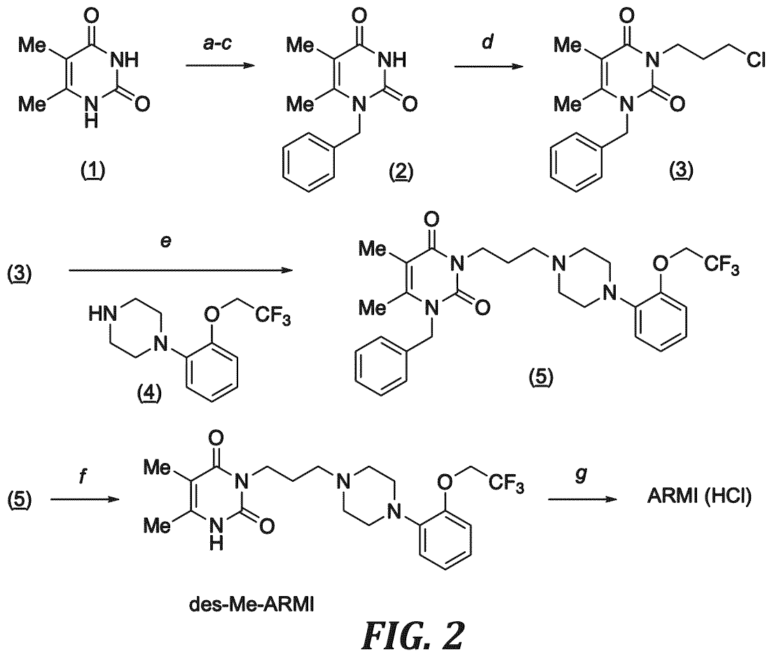

ARMI was synthesized according to the overall reaction scheme illustrated in FIG. 2. In this regard, FIG. 2 describes the synthesis of ARMI including intermediates and relevant reaction conditions. Each step of the synthesis of ARMI is described in more detail below. A radiolabeled isotope of ARMI was also synthesized as described below.

### Preparation of the 1,5,6-Trimethyl-2,4(1H,3H)-Pyrimidinedione Starting Material (FIG. 2, Species (0.1))

Under argon, a room temperature, stirred suspension of 5,6-dimethyluracil (10.00 g, 71 mmol) in HMDS (100 mL) was cautiously treated with concentrated H₂SO₄ (5 drops), then refluxed overnight. Subsequently, the volatile materials were atmospherically distilled (bath 145° C.), under argon, to afford a residual oil. The oil was further concentrated (20 min, 115° C., 2.4 Torr), then allowed to air-cool and crystallize under vacuum. A preliminary 15 mmol scale reaction, with air-bath, vacuum distillation (<1 Torr) of the solid residue (mp~110° C.), provided>95% yield of the NMR-pure bis-OTMS-5,6-dimethyluracil, which is quite sensitive to atmospheric moisture.

Under argon, the vacuum dried (1 Torr/100° C.), crude bis-OTMS-5,6-dimethyluracil (solid) was treated, in situ, with a solution of iodomethane (29.8 g, 1.5 eq) in dry MeCN (100 mL) and the mixture was heated overnight (oil bath 60° C.). Subsequently, the volatile materials were atmospherically distilled off and the residual oil was further concentrated under vacuum (110° C./2 Torr, 30 min). The crude oil was treated with MeOH (200 mL), which rapidly produced a suspension. After 10 min of agitation, the collected solid was rinsed with MeOH and air-dried. The tan colored solid was suspended in water and thiosulfate (100 mg) was added portion-wise to decolorize the mixture. The collected solid was washed liberally with water. The air-dried material (17.65 g) was vacuum dried (125° C./0.5 Torr, 4 h) to provide 14.45 g (mp>215° C.) of a white solid. The process also produced some sublimed colorless crystals (0.14 g, mp~250° C.). The larger mass was recrystallized (EtOH) to provide 11.14 g (51% yield), with mp 255.0-256.7° C. The mother liquors were concentrated and recrystallized (EtOH) to further provide 1.55 g, with mp 252-254° C. The NMR-spectral features (¹H/¹³C) of (1) were consistent with the mono-N-methylated-5,6-dimethyluracil assignment. The measured melting point for (1) markedly differs from literatures values [31111-40-7]: mp's (EtOH) 172-174° C.; >300° C., and for the alternative N(3)methyl regio-isomer [31408-09-0]: 220.5-222° C. (water).

¹H-NMR (500 MHz, d⁶-DMSO) d: 1.79 (3H, s), 2.20 (3H, s), 3.26 (3H, s), 11.135 (1H, s).

¹³C-NMR (125 MHz, d⁶-DMSO) d: 10.71, 16.35, 30.63, 105.71, 149.70, 151.03, 163.16.

HRMS(ESI⁺) m/z: [M+H]⁺ calcd for C₇H₁₁N₂O₂, 155.0821; found, 155.0815.

The schematic reaction chart below depicts the steps of the synthesis of the 1,5,6-Trimethyl-2,4 (1H,3H)-pyrimidinedione (1) starting material.

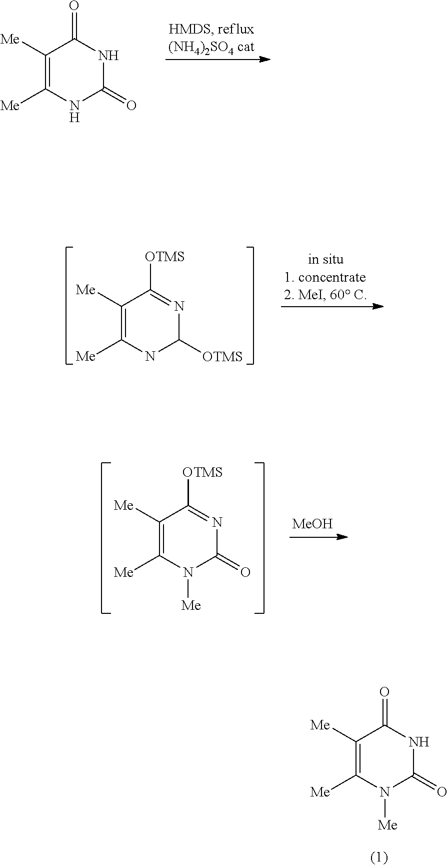

### Preparation of 5,6-Dimethyl-1-(Phenylmethyl)-2,4(1H,3H)-Pyrimidinedione (FIG. 2, Species (2))

As for the synthesis of compound (2), a similarly scalcd reaction was undertaken. Under argon, crude bis-OTMS-5,6-dimethyluracil (107 mmol) was treated, in situ, with a solution freshly distilled benzyl bromide (21.05 g, 1.15 eq) in dry MeCN (100 mL), and the mixture was refluxed for 24 h. The volatile materials were atmospherically distilled off and the residual oil was further concentrated under vacuum (110° C./2 Torr, 30 min). The crude oil was treated with MeOH (300 mL) and the deposited gummy mass was manually worked until a white suspension had fully formed. The solid was washed liberally with MeOH and air-dried. The filtrate and washings were concentrated to a slush. The slush was diluted (portion-wise) with dry ether (150 mL) and the suspension was filtered. The collected solid was liberally washed with ether and air-dried. The combined, isolated solids were vacuum dried (125° C./0.6 Torr, 20 min) to provide 18.19 g (74% yield): mp 198.0-199.9° C. A previous batch of recrystallized material (10% iPrOH/MeCN) exhibited mp 200.0-200.6° C.

¹H-NMR (500 MHz, d⁶-DMSO) δ: 1.81 (3H, s), 2.08 (3H, s), 5.10 (2H, s), 7.17 (2H, d, 7.5 Hz), 7.24 (1H, t, J 7.3 Hz), 7.33 (2H, t, 7.3 Hz), 11.40 (1H, s).

¹³C-NMR (125 MHz, d⁶-DMSO) δ: 10.74, 16.06, 46.38, 106.82, 125.89, 127.14, 128.74, 137.36, 149.02, 151.45, 163.23.

HRMS(ESI⁺) m/z: [M+H]+ calcd for C₁₃H₁₄N₂O₂, 231.11280; found, 231.11206.

The schematic reaction chart below depicts the steps of the synthesis of the 5,6-Dimethyl-1-(phenylmethyl)-2,4(1H,3H)-pyrimidinedione (2) material.

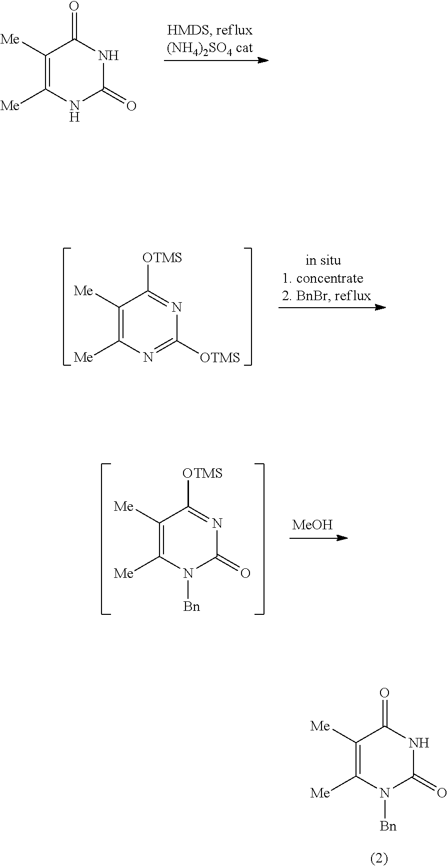

### Preparation of 3-(3-Chloropropyl)-5,6-Dimethyl-1-Methyl-2,4(1H,3H)-Pyrimidinedione (FIG. 2, Species (3))

Under argon, a stirred mixture of (1) (10.00 g, 64.86 mmol), potassium carbonate (44.82 g, 324.3 mmol), benzyl triethylammonium chloride (1.48 g, 6.49 mmol) and 3-iodo-1-chloropropane (39.78 g, 194.6 mmol), in dry DMF (65 mL), was stirred at room temperature for 16 h. The reaction was monitored by TLC ((3) R_{f} 0.69; SiO₂: 10% MeOH/DCM). The mixture was then poured into a stirred mix of water and hexanes (7:1, 800 mL), which precipitated a solid after 5 min. The collected solid was successively washed with water (50 mL) and hexanes (50 mL), then dissolved in EtOAc (100 mL). Separately, the aqueous portion of the filtrate was extracted with hexane (100 mL). The combined hexanes solutions were discarded. Additionally, the aqueous layer was extracted with 1:1 ethyl acetate/hexanes (5×100 mL). All EtOAc containing solutions were combined, then dried (MgSO₄) and concentrated to provide a pale-yellow solid. The crude product was recrystallized Tol/Hex (2:3, 100 mL). The solid was washed with Tol/Hex (1:9, 50 mL), then dried (30° C., <5 Torr) to provide white needles 9.56 g, with mp 83.9-84.6° C. A second crop was recrystallized from Tol (6 mL) layered with Hex (20 mL). The crop was washed with 5% Et₂O/Hex, to provide 2.30 g of pale-yellow crystals, with mp 82.5-83.5° C. The combined yield was 11.86 g (79%).

¹H-NMR (500 MHz, CDCl₃) δ: 1.98 (3H, s), 2.12 (2H, pent, J 6.8 Hz), 2.28 (3H, s), 3.45 (3H, s), 3.57 (2H, t, J 6.8 Hz), 4.10 (2H, t, J 6.8 Hz).

¹³C-NMR (125 MHz, CDCl₃) δ: 11.57, 16.61, 30.84, 32.03, 39.48, 42.59, 106.94, 147.09, 151.71, 162.83.

HRMS(ESI⁺) m/z: [M+H]⁺ calcd for C₁₀H₁₅C₁N₂O₂, 231.08948; found, 231.08830.

The schematic reaction chart below depicts the steps of the synthesis of the 5,6-Dimethyl-1-(phenylmethyl)-2,4(1H,3H)-pyrimidinedione (3) material.

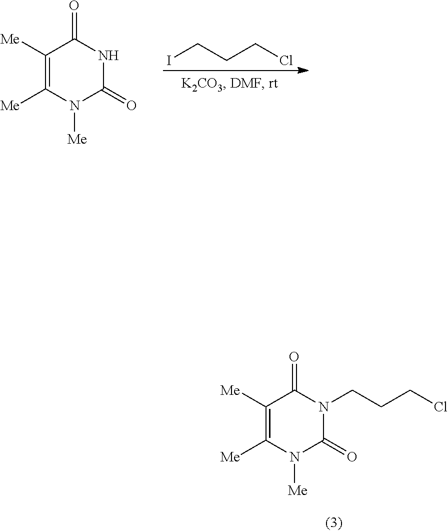

### Preparation of 3-(3-Chloropropyl)-5,6-Dimethyl-1-(Phenylmethyl)-2,4(1H,3H)-Pyrimidinedione (FIG. 2, Species (4); 186387-58-6)

As for the synthesis of (4), a scalcd reaction of (2) (15.0 g, 65 mmol, 19 h, room temperature) provided crude (4), which was recrystallized from Tol/Hex (1:5, 120 mL) to afford 16.48 g (82% yield), with mp 92.3-93.3° C.

¹H-NMR (500 MHz, CDCl₃) δ: 1.97 (3H, s), 2.17 (3H, s), 2.17 (2H, pent), 3.59 (2H, t, J 6.8 Hz), 4.16 (2H, t, J 7.0 Hz) 5.18 (2H, s), 7.16 (2H, d, J 7.2 Hz), 7.27 (1H, t, J 7.0 Hz), 7.34 (2H, t, J 7.6 Hz).

¹³C-NMR (125 MHz, CDCl₃) δ: 11.17, 16.44, 30.98, 39.88, 42.70, 48.31, 107.79, 126.12, 127.70, 129.05, 136.57, 147.08, 152.21, 162.95.

HRMS(ESI⁺) m/z: [M+H]⁺ calcd for C₁₆H₁₉ClN₂O₂, 307.12078; found, 307.11829.

The schematic reaction chart below depicts the steps of the synthesis of the 3-(3-Chloropropyl)-5,6-dimethyl-1-(phenylmethyl)-2,4(1H,3H)-pyrimidinedione (4) material.

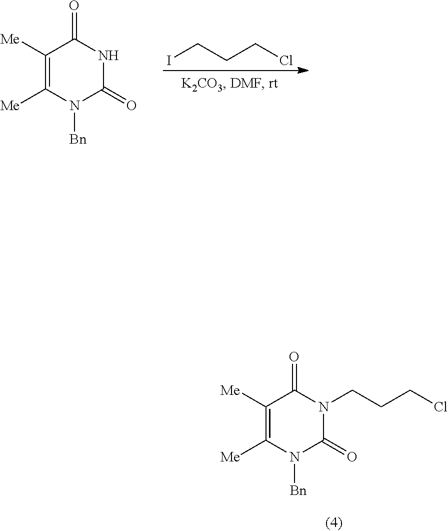

### Preparation of 5,6-Dimethyl-1-(Phenylmethyl)-3-[3-[4-[2-(2,2,2-Trifluoroethoxy)Phenyl]-1-Piperazinyl]Propyl]-2,4(1H,3H)-Pyrimidinedione (FIG. 2, Species (5))

Under argon, a mixture (4) (14.81 g, 48.27 mmol), 1-[2-(2,2,2-trifluoroethoxy)phenyl]piperazine dihydrochloride (as shown in synthesis of 7) (20.9 g, 62.76 mmol), potassium carbonate (20.01 g, 144.8 mmol) and potassium iodide (40.1 g, 241.4 mmol), in dry MeCN, (150 mL), was refluxed for 22 h. After cooling to room temperature, EtOAc (150 mL) was added, and the mixture was stirred and filtered. The filtrate was concentrated in vacuo. The collected solid was taken up into hot EtOAc/Hex (1:1, 60 mL) and filtered once more. The combined filtrates were passed through a wide column of silica gel (150 mL, previously equilibrated with 10% EtOAc/Hex. Subsequently, the column was eluted with 50% EtOAc/Hex, then EtOAc. Selected column fractions were combined and concentrated to afford a vacuum dried, white crystalline solid, 17.99 g (70% yield), with mp 100.4-101.4° C.

¹H-NMR (500 MHz, CDCl₃) δ: 1.92 (2H, pent, J 7.2 Hz), 1.98 (3H, s), 2.15 (3H, s), 2.51 (2H, t, J 6.9 Hz), 2.62 (4H, s (br, 60 Hz), 3.08 (4H, s (br, 60+Hz), 4.11 (2H, dd, 14.8 Hz wide), 4.39 (2H, q, ³J_{HF} 8.4 Hz), 5.17 (2H, s (br, 25+Hz), 6.89 (1H, dd, J 8.4, 1.4 Hz), 6.936 (2H, m), 7.03 (1H, td, J 8.3, 1.6 Hz), 7.16 (2H, d(br), 7.2 Hz), 7.26 (1H, m), 7.33 (2H, d, 7.8 Hz).

¹³C-NMR (125 MHz, CDCl₃) δ: 11.76, 16.33, 24.85, 40.46, 48.18, 50.63, 53.48, 56.08, 66.79 (q, ²J_{CF} 35.5 Hz), 107.78, 116.87, 118.93, 122.64, 123.67 (q, ¹J_{CF} 278.1 Hz), 124.08, 126.08, 127.62, 128.98, 136.67, 143.02, 146.67, 150.27, 152.21, 162.98.

HRMS(ESI⁺) m/z: [M+H]⁺ calcd for C₂₈H₃₃F₃N₄O₃, 531.25775; found, 531.25404.

The compound structure of the 5,6-Dimethyl-1-(phenylmethyl)-3-[3-[4-[2-(2,2,2-trifluoroethoxy)phenyl]-1-piperazinyl]propyl]-2,4(1H,3H)-pyrimidinedione (5) material is provided below.

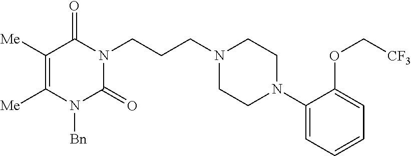 [SMILES: Cc1c(C)n(Cc2ccccc2)c(=O)n(CCCN2CCN(c3ccccc3OCC(F)(F)F)CC2)c1=O]

### Preparation of 5,6-Dimethyl-3-[3-[4-[2-(2,2,2-Trifluoroethoxy)Phenyl]-1-Piperazinyl]Propyl]-2,4(1H,3H)-Pyrimidinedione (FIG. 2, Species (6); Des-Methyl-ARMI)

Under argon, a mixture of (5_) (17.71 g, 33.38 mmol) and NH₄HCO₂ (21.05 g, 334 mmol), in methanol (180 mL), was chilled and a slurry of 5 wt % Pd/C (1.78 g) in MeOH (40 mL) was added. The reaction mixture was subsequently refluxed for 18 h. The mixture was treated with Celite (10 g) and EtOAc (200 mL) at RT, and then filtered through Celite (25 g). The filtered solids were rinsed with water (50 mL) and the combined filtrates were concentrated to a white residue. The filter cake was further rinsed with water (100 mL) and EtOAc (100 mL). The biphasic filtrate was combined with the white solid and saturated bicarbonate (20 mL) was added. The mixture was partitioned, and the aqueous phase was further extracted with EtOAc (3×100 mL). The combined organic solutions were washed with brine, dried (MgSO₄) and concentrated to a crystalline solid (14.4 g), which was recrystallized from hot MeCN (50 mL). The crystals were successively washed with MeCN (50 mL), EtOAc (10 mL) and Hex (20 mL), followed by vacuum drying, to provide colorless crystals (11.14 g), with mp 155.9-157.3° C. A second crop was recrystallized from MeCN (12 mL) to provide 1.52 g of large translucent crystals, with 155.1-156.5° C. The combined yield of (6) was 12.66 g (86%), as the free-base.

1H-NMR (500 MHz, CDCl₃) δ: 1.88 (2H, pent, J 7.1 Hz), 1.92 (3H, s), 2.16 (3H, s), 2.51 (2H, t, J 7.0 Hz), 2.62 (4H, s (br, 60 Hz)), 3.08 (4H, br s, 60 Hz wide), 4.03 (2H, dd, 14.8 Hz wide), 4.39 (2H, dd, J 16.7, 8.4 Hz), 6.90 (1H, dd, J 7.7, 1.2 Hz), 6.95 (2H, m (21.1 Hz wide)), 7.04 (1H, td, 7.6, 1.6 Hz), 10.72 (1H, s).

¹³C-NMR (125 MHz, CDCl₃) δ: 10.55, 16.67, 24.79, 39.35, 50.60, 53.36, 55.98, 66.70 (q, J 35.1 Hz), 106.52, 116.59, 118.88, 122.73, 123.66 (q, J 278.3 Hz), 124.07, 142.87, 144.62, 150.16, 152.98, 164.03.

¹⁹F-NMR (470 MHz, CDCl₃) δ: −73.96.

HRMS(ESI⁺) m/z: [M+H]⁺ calcd for C₂₁H₂₇F₃N₄O₃, 441.2108; found, 441.2071.

The compound structure for 5,6-Dimethyl-3-[3-[4-[2-(2,2,2-trifluoroethoxy)phenyl]-1-piperazinyl]propyl]-2,4(1H,3H)-pyrimidinedione is provided below. (6; des-methyl-ARMI) material.

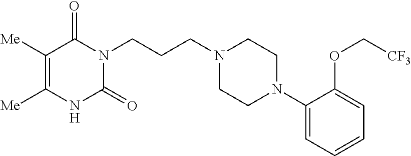 [SMILES: Cc1[nH]c(=O)n(CCCN2CCN(c3ccccc3OCC(F)(F)F)CC2)c(=O)c1C]

### Preparation of 1,5,6-Trimethyl-3-[3-[4-[2-(2,2,2-Trifluoroethoxy)Phenyl]-1-Piperazinyl]Propyl]-2,4(1H,3H)-Pyrimidinedione (FIG. 2, Species (7); ARMI)

### Method A

Under argon, a room temperature mixture of (6) (5.00 g, 11.35 mmol), Cs₂CO₃ (7.40 g, 22.70 mmol), and dry DMF (23 mL) was stirred for 20 minutes, then treated, in one portion, with methyl iodide (1.77 g, 12.49 mmol). After 2 h, the mixture was poured into water (400 mL) and the solution was extracted with 1:1 EtOAc/Hex (5×100 mL). The combined organic layers were washed with brine (100 mL), dried with MgSO₄, and concentrated to a residual yellow oil. The oil was dissolved in DCM (100 mL) and the solution was washed with 2M NaOH (2×25 mL) and brine (25 mL), then dried (MgSO₄) and evaporated to an off-white gummy foam, which crystallized on standing (4.33 g). This material was taken up into dry dioxane (20 mL) and the mixture treated with 4N hydrogen chloride (2.3 mL, 1 eq) in dioxane. The salt was precipitated with Et₂O (100 mL) and the solvents were evaporated. The amine-salt was recrystallized from MeCN (12 mL). A small crystal sample was assayed by HPLC (>98% pure). The remaining crystals and mother liquor were basified with NaHCO₃ (100 mL) and the mixture was extracted with EtOAc (3×100 mL). The dried and concentrated extract afforded an oil. The material was dry loaded on silica gel (10 g), then chromatographed over silica gel (80 g), gradient eluted with EtOAc/Hex (85-100%). Concentration of selected fractions afforded (7) (2.72 g, 53% yield) as white crystals, with mp 110.6-111.2° C. For long-term storage, (2) was subsequently converted to its mono-hydrochloride salt. (7) (2.60 g) was in i-PrOH (10 mL), was treated with 4N HCl-dioxane (1.5 mL, 1 eq). Removal of the volatile materials gave a white solid, after a final evaporation from MeCN. The solid was crystallized from hot MeCN (8 mL) to afford (2-HCl) (2.52 g) after vacuum drying (<1 Torr/60° C./1 h). The melting point of the prepared material, 216.3-217.3° C., was recorded after prematurely melting the sample (out-gassing), letting it cool, to resolidify, followed by a final remelt. Fully out-gassed crystalline (7-HCl) was obtained after further vacuum drying the bulk material at 125° C. (0.5 Torr/5 h), mp 216.3-217.3° C.

The schematic reaction chart below depicts the steps of the synthesis of the 1,5,6-Trimethyl-3-[3-[4-[2-(2,2,2-trifluoroethoxy)phenyl]-1-piperazinyl]propyl]-2,4(1H,3H)-pyrimidinedione (2; ARMI) material.

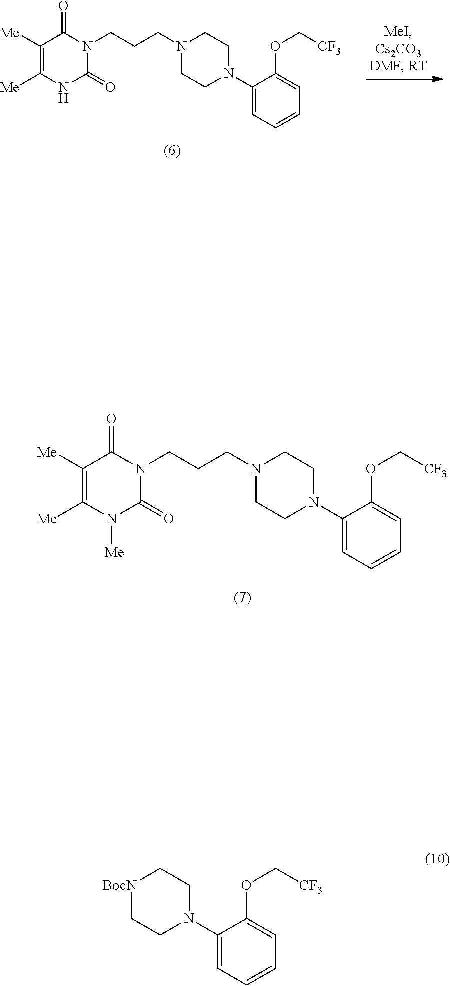 [SMILES: Cc1[nH]c(=O)n(CCCN2CCN(c3ccccc3OCC(F)(F)F)CC2)c(=O)c1C ; Cc1c(C)n(C)c(=O)n(CCCN2CCN(c3ccccc3OCC(F)(F)F)CC2)c1=O ; CC(C)(C)OC(=O)N1CCN(c2ccccc2OCC(F)(F)F)CC1]

### Method B

Under argon, a stirred mixture of (3) (1.30 g, 5.7 mmol), (11-2 HCl) (2.65 g, 1.4 eq), potassium carbonate (3.5 g, 3 eq), and potassium iodide (4.7 g, 4 eq), in dry MeCN (40 mL) was refluxed overnight to complete the reaction of (3) (HPLC). Excess (11) was converted to its N-Boc-derivative (10) by treatment of the reaction mixture with BOC₂O (0.75 g). The mixture was diluted with EtOAc (50 mL), filtered and the filtrate was evaporated. The residue was taken up into EtOAc/Hex (2.1). (7-HCl) was extracted out by washing with 0.5 M HCl aq (3×100 mL). During the extraction, some (7-HCl) formed, as white crystals. The combined aqueous extract and crystals were treated with portions of bicarbonate to achieve pH 8. (7) was extracted out with successive portions of EtOAc (total 125 mL). The combined extract was successively washed with bicarbonate and brine, then dried (MgSO₄). Concentration gave and oil, which crystallized on standing to provide 3.04 g. Recrystallization of the solid from hot i-PrOH (10 mL) to afford (7), as white rectangular prisms, with mp 110.6-111.7° C., which was the same for (7) prepared by Method A (mp 110.6-111.2° C.), and identical by HPLC.

The schematic reaction chart below depicts the steps of the synthesis of the 1,5,6-Trimethyl-3-[3-[4-[2-(2,2,2-trifluoroethoxy)phenyl]-1-piperazinyl]propyl]-2,4(1H,3H)-pyrimidinedione (7; ARMI) material.

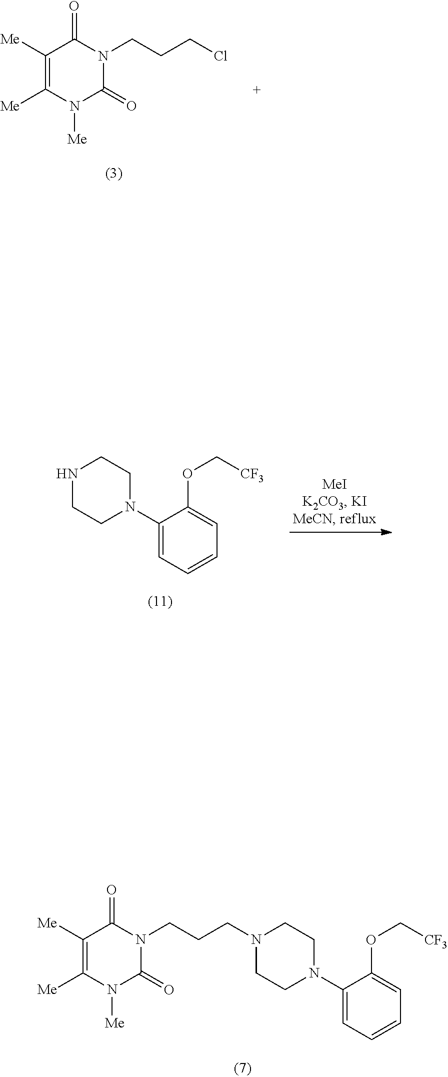 [SMILES: Cc1c(C)n(C)c(=O)n(CCCCl)c1=O ; FC(F)(F)COc1ccccc1N1CCNCC1 ; Cc1c(C)n(C)c(=O)n(CCCN2CCN(c3ccccc3OCC(F)(F)F)CC2)c1=O]

### Spectroscopic Characterization of ARMI

(7): ¹H-NMR: (500 MHz, d⁶-DMSO) d: 1.88 (2H, pent, average J 7.3 Hz, —CH₂CH₂CH₂—), 1.99 (3H, s, C₅-Me), 2.24 (3H, s, C₆-Me), 2.51 (2H, t, J 7.2 Hz, N3-CH₂CH₂CH₂—N), 2.63 (4H, br s, 28 Hz, —CH₂N—), 3.09 (4H, br s, 30 Hz, —CH₂NAr), 3.43 (3H, s, N1-Me), 4.05 (2H, dd, average J 7.3 Hz, N3-CH₂—), 4.39 (2H, q, ²J_{HF} 8.3 Hz, —CH₂CF₃), 6.89 (1H, m, ArH), 6.95 (2H, m, ArH), 7.04 (1H, m, ArH).

¹³C NMR: (125 MHz, d⁶-DMSO) d: 11.71 (uracil(C6)-methyl), 16.62 (uracil(C5)-methyl), 24.69 (uracil(N3)-CH₂CH₂CH₂—), 32.04 (uracil(N1)-methyl), 40.31 (CH₂CH₂CH₂N), 50.58 (2×CH₂—NAr), 53.32 (2×NCH₂(piperazine ring), 55.99 (uracil(N3)-CH₂CH₂CH₂—), 66.67 (CH₂CF₃ (q, ²J_{CF} 35.1 Hz), 107.13 (uracil(C5) a-vinyl), 116.67 (ArCH: ortho-to oxygen), 118.86 (ArCH: meta (or meta′) to oxygen), 122.61 (ArCH: meta (or meta′) to oxygen), 123.63 (CF₃ (q, ¹J_{CF} 227.8 Hz), 124.02 (ArCH: para-O), 142.91 (ArC—N), 146.67 (uracil(C6) b-vinyl), 150.13 (ArC—OR), 151.89 (uracil(C₂)-carbonyl), 160.03 (uracil(C4)-carbonyl).

¹⁹F-NMR (470 MHz, CDCl₃) d −73.96 ppm.

HRMS(ESI⁺) m/z: [M+H]⁺ calcd for C₂₂H₃₀N₄O₃F₃ 455.2264; found 455.2261.

HPLC: Phenomenex-Gemini column (100×3 mm, 3 micron particle C18); eluted with 45% MeCN/55% 25 mM NH₄OAc (pH 8.5); 1 mL/min; UV (280 nm), 30° C.; Rt 3.16 min.

### Spectroscopic Characterization of the HCL Salt of ARMI

(7-HCl): ¹H-NMR: (500 MHz, d⁶-DMSO) d: 1.88 (3H, s, C(5)-Me), 2.06 (2H, m, CH₂—CH₂—CH₂), 2.25 (3H, s, C(6)Me), 3.06-3.23 (6H, overlapping multiplets, piperazine ring (4H) and alkyl-CH₂—N(piperazine)), 3.36 (3H, s, NMe), 3.436 (2H, br d, 11.9 Hz, piperazine ring), 3.52 (2H, br d, 10.9 Hz, piperazine), 3.89 (2H, t, 6.9 Hz, N(3)CH₂—), 4.74 (2H, q, ³J_{HF} 8.8 Hz, ArOCH₂CF₃), 6.96-7.04 (3H, m, ArH), 7.06 (1H, m, ArH) 11.52 (1H, br s, R₃NH⁺).

¹³C NMR: (125 MHz, d⁶-DMSO) d: 11.40, 16.39, 21.95, 31.81, 38.45, 46.71, 50.90, 53.27, 65.30 (q, ²J_{CF} 33.0 Hz), 105.11, 115.14, 118.74, 123.07, 123.26, 124.10 (q, ¹J_{CF} 273.0 Hz), 140.19, 148.60, 149.38, 151.15, 162.16.

¹⁹F NMR: (470 MHz, d⁶-DMSO) d: −72.587.

HRMS(ESI⁺) m/z: [M]⁺ calcd for C₂₂H₃₀F₃N₄O₃, 455.2264; found 455.2262.

Radiolabeling of ARMI with [¹¹C]MEI to Form [C-11]ARMI

Radiolabeling of ARMI with [C-11]MeI was performed stepwise:
(1) production of [C-11]CO₂ by 11-MeV proton bombardment of pressurized nitrogen (99.9999% N₂/1-2% oxygen) gas;
[symbol] Connversion of delivered [C-11]CO₂, into [C-11]MeI, using a [symbol] (e.g. GE TracerLab FX2 MeI);
[symbol] er of [C-11]MeI/He, into a solution of des-Me-ARMI (0.5-1.6 [symbol] e, ~150 uL), basified by Cs₂CO₃ pre-treatment, followed by a [symbol] nin);
[symbol] ,C purification of [C-11]ARMI (1:1 MeCN/0.1M NH₄OH-pH
(5) solid phase extraction (C18-SPE) of HPLC-captured [C-11]ARMI;
(6) reformulation of [C-11]ARMI by sequential elution of the C18-SPE with: USP-water, air, EtOH and USP normal saline, followed by collective terminal sterilization (0.2 um micron-filtration).

The schematic reaction chart below depicts the steps of the synthesis of the radiolabeled ARMI.

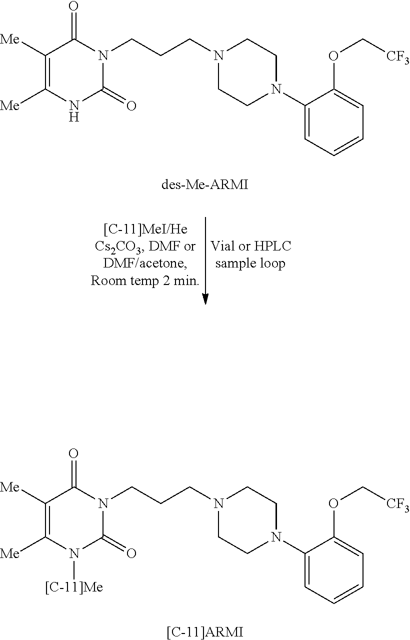

Typically, >5.6 GBq (150 mCi) of [C-11]ARMI (HPLC-collection) was available (e.g. <45 min), from 74 GBq (2 Ci) of [C-11]CO₂ (EOB), with a specific activity (Aₘ, 148-185 GBq/umol (4-8 Ci/umol), EOB). The methylation reaction chamber was either a septum sealed, low volume glass vial (e.g. 1 mL), or a similarly charged empty stainless-steel HPLC sample injection loop (e.g. 2-5 mL, 1/16 in OD×0.04 ID), interconnected with the semi-preparative HPLC column.

Radiolabeling with [18F]Fluoride to Form [¹⁸F] Compound 12

Radiolabeling of [¹⁸F] Compound 12 with [¹⁸F]fluoride was performed stepwise:
(1) production of [¹⁸F]fluoride by proton bombardment of ¹⁸O-enriched water;
(2) capture of [¹⁸F]fluoride by pushing the target water through a Chromafix ¹⁸F separation cartridge with helium pressure using a GE Tracerlab FXFN automated synthesis unit;
(3) elution of the concentrated ¹⁸F⁻ off the cartridge with aqueous potassium carbonate into the reaction vessel;
(4) azeotropically drying the eluted fluoride with a solution of Kryptofix[2,2,2] in anhydrous acetonitrile under heat and reduced pressure into the reaction vessel;
(2) addition of 100 μL of a 20 mg/mL solution of labeling precursor in anhydrous DMSO and heating the mixture for 20-25 min at 120 to 140° C.;
(4) C-18 HPLC purification of [¹⁸F] Compound 12 (6:4 MeCN/0.1M NH₄OH-pH 9.6 (HOAc);
(5) solid phase extraction (C18-SPE) of HPLC-captured [¹⁸F] Compound 12;
(6) reformulation of [¹⁸F] Compound 12 by sequential elution of the C18-SPE with: USP-water, air, EtOH and USP normal saline, followed by collective terminal sterilization (0.2 um micron-filtration).

The schematic reaction chart below depicts the steps of the synthesis of the radiolabeled Compound 12.

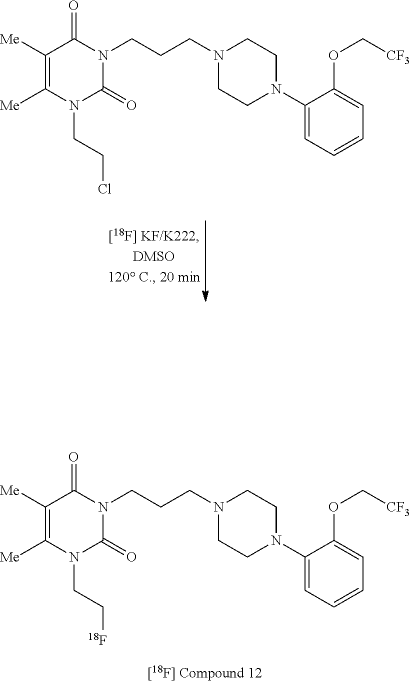

Typically, >2 GBq (50 mCi) of [¹⁸F] Compound 12 (HPLC-collection) was available (<45 min), from 11 GBq (300 mCi) of [¹⁸F]fluoride (SOS), with a specific activity (Aₘ, 40-90 GBq/umol (1-2.5 Ci/umol), EOS). The reaction vessel was typically a low volume glass vial (e.g. 2 mL) with conical point to facilitate drying.

The synthesis of the labeling precursor follows.

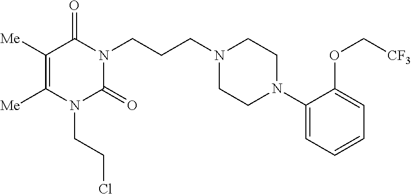 [SMILES: Cc1c(C)n(CCCl)c(=O)n(CCCN2CCN(c3ccccc3OCC(F)(F)F)CC2)c1=O]

5,6-Dimethyl-3-[3-[4-[2-(2,2,2-trifluoroethoxy)phenyl]-1-piperazinyl]propyl]-2,4(1H,3H)-pyrimidinedione (628.2 mg, 1.43 mmol) and cesium carbonate (929.3 mg, 2.85 mmol) were combined in a 25 mL RBF with magnetic stir bar. The flask was purged under argon for one minute then DMF (4.8 mL) was added. The suspension stirred for 15 minutes at 60° C. 2-Chloroethyl p-toluenesulfonate (298 μL, 1.64 mmol) was added in one portion and the reaction continued to stir at 60° C. for 90 minutes.

The reaction mixture was poured into 100 mL of water. The mixture was extracted with 4×50 mL portions of 1:1 ethyl acetate/hexanes. The combined organic layers were dried with MgSO4, filtered, and concentrated under reduced pressure.

The residue was purified with 40:60:1 ethyl acetate/hexanes/triethylamine and a 25 g silica gel cartridge. The product was collected as a white solid (365.2 mg, 0.726 mmol, 51% yield).

¹H NMR (500 MHz, CDCl₃) δ 7.10-7.00 (m, 1H), 6.97-6.91 (m, 2H), 6.90-6.71 (m, 1H), 4.38 (q, J=8.4 Hz, 2H), 4.17 (t, J=6.4 Hz, 2H), 4.02 (t, J=7.5 Hz, 2H), 3.77 (t, J=6.4 Hz, 2H), 3.07 (br s, 4H), 2.60 (br s, 4H), 2.48 (t, J=7.2 Hz, 2H), 2.30 (s, 3H), 1.99 (s, 3H), 1.86 (p, J=7.4 Hz, 2H).

¹³C NMR (126 MHz, CDCl₃) δ 162.95, 151.56, 150.25, 146.12, 142.99, 124.13, 123.72 (q, J=278.4 Hz), 122.75, 119.00, 116.75, 107.97, 66.79 (q, J=35.0 Hz), 56.10, 53.46, 50.68, 46.66, 41.15, 40.33, 24.80, 16.83, 12.06.

### Synthesis of Other Exemplary Compounds

In an aspect, radiolabeled compounds according to embodiments of the present disclosure may be synthesized according to the above reaction conditions and according to the reaction schematic for Reaction I, presented below.

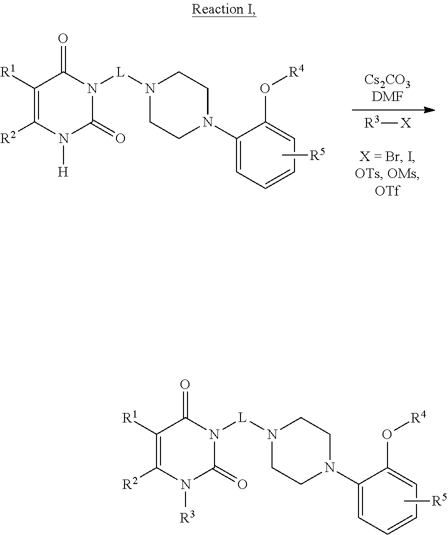

wherein R³ is a substituent selected from the group consisting of: a carbon-11 (¹¹C), carbon-14 (¹⁴C), or tritium (³H) labeled C₁-C₆ alkyl; a carbon-11 (¹¹C), carbon-14 (¹⁴C), tritium (3H), or fluorine-18 (¹⁸F) labeled C₁-C₆ haloalkyl; a carbon-11 (¹¹C), carbon-14 (¹⁴C), or tritium (³H) labeled C₁-C₆ hydroxyl alkyl; a carbon-11 (¹¹C), carbon-14 (¹⁴C), or tritium (³H) labeled C₃-C₆ cycloalkyl; a carbon-11 (¹¹C), carbon-14 (¹⁴C), or tritium (³H) labeled C₁-C₆ alkylnitrile; and a carbon-11 (¹¹C), carbon-14 (¹⁴C), or tritium (³H) labeled C₁-C₆ alkylcycloalkyl.

Compounds according to embodiments of the present disclosure are provided in Table 1 below.

TABLE 1
Exemplary Compounds

| Com- | |
|---|---|
| pound | |
| # | Structure |
| 0 | 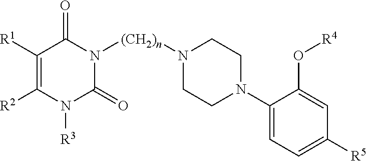 |
| 1 | 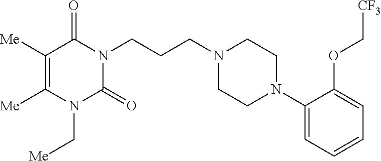 [SMILES: CCn1c(C)c(C)c(=O)n(CCCN2CCN(c3ccccc3OCC(F)(F)F)CC2)c1=O] |
| 2 | 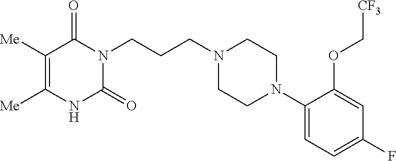 [SMILES: Cc1[nH]c(=O)n(CCCN2CCN(c3ccc(F)cc3OCC(F)(F)F)CC2)c(=O)c1C] |
| 3 | 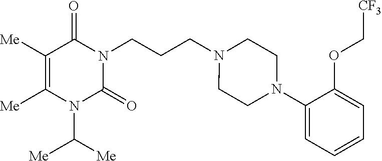 [SMILES: Cc1c(C)n(C(C)C)c(=O)n(CCCN2CCN(c3ccccc3OCC(F)(F)F)CC2)c1=O] |
| 4 | 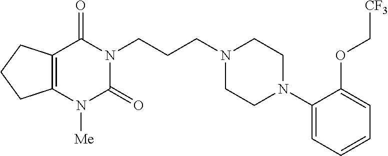 [SMILES: Cn1c2c(c(=O)n(CCCN3CCN(c4ccccc4OCC(F)(F)F)CC3)c1=O)CCC2] |
| 5 | 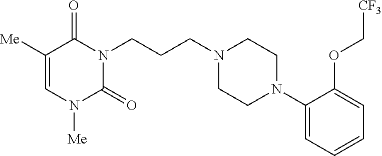 [SMILES: Cc1cn(C)c(=O)n(CCCN2CCN(c3ccccc3OCC(F)(F)F)CC2)c1=O] |
| 6 | 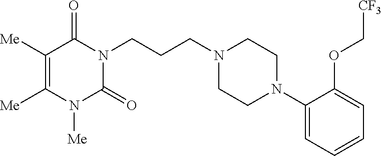 [SMILES: Cc1c(C)n(C)c(=O)n(CCCN2CCN(c3ccccc3OCC(F)(F)F)CC2)c1=O] |
| 7 | 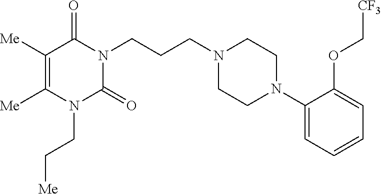 [SMILES: CCCn1c(C)c(C)c(=O)n(CCCN2CCN(c3ccccc3OCC(F)(F)F)CC2)c1=O] |
| 8 | 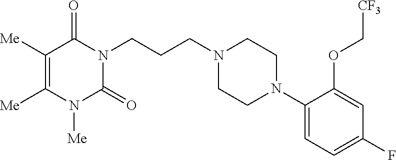 [SMILES: Cc1c(C)n(C)c(=O)n(CCCN2CCN(c3ccc(F)cc3OCC(F)(F)F)CC2)c1=O] |
| 9 | 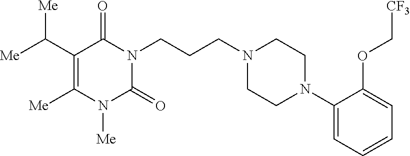 [SMILES: Cc1c(C(C)C)c(=O)n(CCCN2CCN(c3ccccc3OCC(F)(F)F)CC2)c(=O)n1C] |
| 10 | 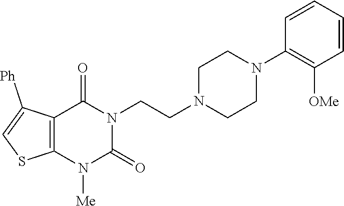 [SMILES: COc1ccccc1N1CCN(CCn2c(=O)c3c(-c4ccccc4)csc3n(C)c2=O)CC1] |
| 11 | 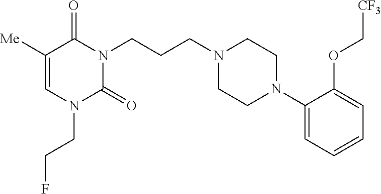 [SMILES: Cc1cn(CCF)c(=O)n(CCCN2CCN(c3ccccc3OCC(F)(F)F)CC2)c1=O] |
| 12 | 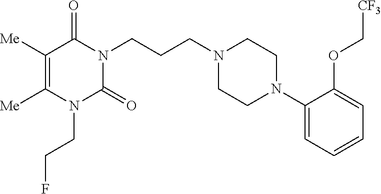 [SMILES: Cc1c(C)n(CCF)c(=O)n(CCCN2CCN(c3ccccc3OCC(F)(F)F)CC2)c1=O] |
| 13 | 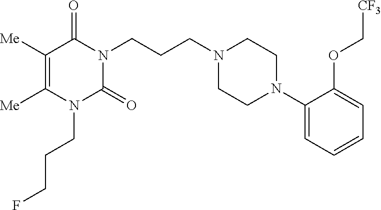 [SMILES: Cc1c(C)n(CCCF)c(=O)n(CCCN2CCN(c3ccccc3OCC(F)(F)F)CC2)c1=O] |
| 14 | 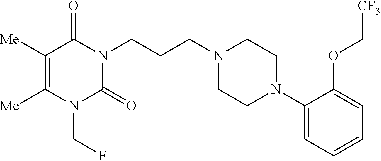 [SMILES: Cc1c(C)n(CF)c(=O)n(CCCN2CCN(c3ccccc3OCC(F)(F)F)CC2)c1=O] |
| 15 | 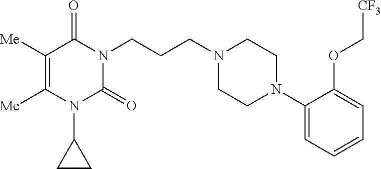 [SMILES: Cc1c(C)n(C2CC2)c(=O)n(CCCN2CCN(c3ccccc3OCC(F)(F)F)CC2)c1=O] |
| 16 | 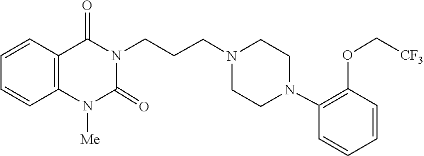 [SMILES: Cn1c(=O)n(CCCN2CCN(c3ccccc3OCC(F)(F)F)CC2)c(=O)c2ccccc21] |
| 17 | 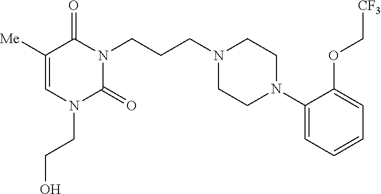 [SMILES: Cc1cn(CCO)c(=O)n(CCCN2CCN(c3ccccc3OCC(F)(F)F)CC2)c1=O] |
| 18 | 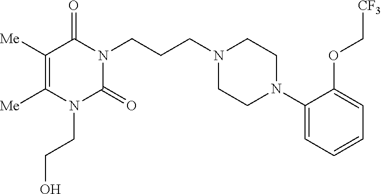 [SMILES: Cc1c(C)n(CCO)c(=O)n(CCCN2CCN(c3ccccc3OCC(F)(F)F)CC2)c1=O] |
| 19 | 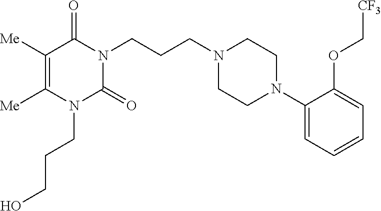 [SMILES: Cc1c(C)n(CCCO)c(=O)n(CCCN2CCN(c3ccccc3OCC(F)(F)F)CC2)c1=O] |
| 20 | 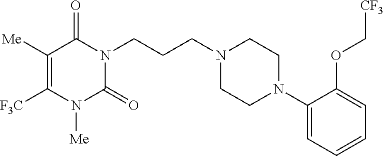 [SMILES: Cc1c(C(F)(F)F)n(C)c(=O)n(CCCN2CCN(c3ccccc3OCC(F)(F)F)CC2)c1=O] |
| 21 | 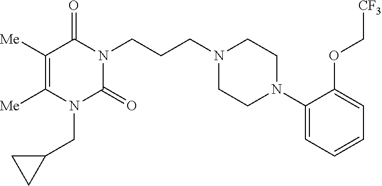 [SMILES: Cc1c(C)n(CC2CC2)c(=O)n(CCCN2CCN(c3ccccc3OCC(F)(F)F)CC2)c1=O] |
| 22 | 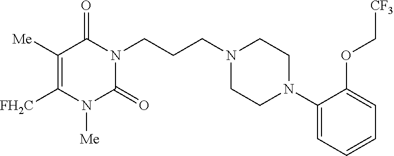 |
| 23 | 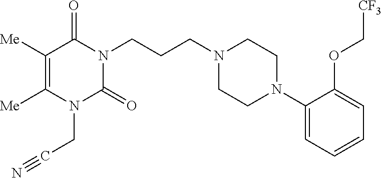 [SMILES: Cc1c(C)n(CC#N)c(=O)n(CCCN2CCN(c3ccccc3OCC(F)(F)F)CC2)c1=O] |

### Synthesis of Compound 1

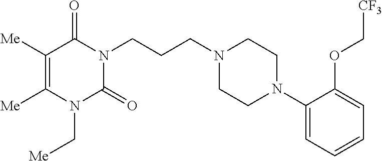 [SMILES: CCn1c(C)c(C)c(=O)n(CCCN2CCN(c3ccccc3OCC(F)(F)F)CC2)c1=O]

5,6-Dimethyl-3-[3-[4-[2-(2,2,2-trifluoroethoxy)phenyl]-1-piperazinyl]propyl]-2,4(1H,3H)-pyrimidinedione (208.2 mg, 0.473 mmol) and cesium carbonate (308.0 mg, 0.945 mmol) were combined in a 10 mL RBF with magnetic stir bar. The flask was purged under argon for one minute then DMF (1.6 mL) was added. The suspension stirred for 10 minutes at room temperature. Iodoethane (43.7 μL, 0.544 mmol) was added in one portion and the reaction continued to stir at room temperature for 2.5 hours.

The reaction mixture was poured into 30 mL of water. The mixture was extracted with 3×30 mL portions of 1:1 ethyl acetate/hexanes. The combined organic layers were dried with MgSO₄, filtered, and concentrated under reduced pressure.

The residue was purified with 20:80:1 to 50:50:1 ethyl acetate/hexanes/triethylamine and a 12 g silica gel cartridge. The product was collected as a thick oil (185.4 mg, 0.396 mmol, 84% yield)

Rf=0.125, 80:20:1 Ethyl acetate/hexanes/triethylamine.

¹H NMR (500 MHz, CDCl₃) δ 6.96 (t, J=7.6 Hz, 1H), 6.90-6.84 (m, 2H), 6.84-6.78 (m, 1H), 4.32 (q, J=8.4 Hz, 2H), 3.97 (t, J=7.5 Hz, 2H), 3.87 (q, J=7.1 Hz, 2H), 3.00 (br s, 4H), 2.54 (br s, 4H), 2.43 (t, J=7.2 Hz, 2H), 2.19 (s, 3H), 1.91 (s, 3H), 1.80 (p, J=7.3 Hz, 2H), 1.20 (t, J=7.2 Hz, 3H).

¹³C NMR (126 MHz, CDCl₃) δ 163.15, 151.49, 150.24, 146.17, 143.01, 124.12, 123.72 (q, J=278.3 Hz), 122.72, 118.99, 116.79, 107.38, 66.79 (q, J=35.1 Hz), 56.15, 53.44, 50.66, 40.46, 40.25, 24.82, 15.97, 14.30, 11.83.

¹⁹F NMR (470 MHz, CDCl₃) δ −73.96 (t, J=8.6 Hz). (unreferenced, 1H coupled)

### Synthesis of Compound 2

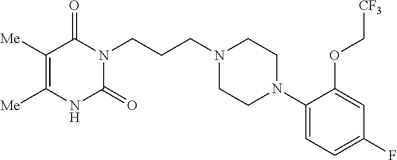 [SMILES: Cc1[nH]c(=O)n(CCCN2CCN(c3ccc(F)cc3OCC(F)(F)F)CC2)c(=O)c1C]

5,6-Dimethyl-1-(phenylmethyl)-3-[3-[4-[2-(2,2,2-trifluoroethoxy)-4-fluorophenyl]-1-piperazinyl]propyl]-2,4(1H,3H)-pyrimidinedione (644.9 mg, 1.18 mmol) and ammonium formate (741.3 mg, 11.8 mmol) were sparged under argon in a 50 mL RBF with a magnetic stir bar and condenser. Methanol (10 mL) was added. At −78° C., 5 wt % palladium on carbon (125.1 mg, 0.0588 mmol Pd) was slurried with chilled methanol (10 mL) and added to the reaction mixture against argon flow. The reaction was refluxed for six hours.

The reaction was chilled to −78° C. Celite (1.5 g), water (5 mL), and ethyl acetate (20 mL) were added to the vessel. The suspension was filtered through celite (15 mL). The filtrate was concentrated under reduced pressure (to remove methanol). Half saturated bicarbonate (50 mL) was added, and the mixture was extracted with 3×50 mL portions of ethyl acetate. The combined organic layers were washed with brine, dried with magnesium sulfate, filtered, and concentrated under reduced pressure.

The obtained solid was recrystallized from hot ethyl acetate (3 mL), collected on filter paper, and washed with chilled ethyl acetate (2 mL) and hexanes (10 mL). The product was obtained as white crystals (405.9 mg, 0.885 mmol, 75% yield).

MP: Melts and resolidifies when soaked at 155° C., and then melts from 170.9-171.6° C.

¹H NMR (500 MHz, CDCl₃) δ 10.79 (s, 1H), 6.85 (dd, J=8.9, 5.8 Hz, 1H), 6.71 (ddd, J=8.8, 7.8, 2.8 Hz, 1H), 6.62 (dd, J=9.5, 2.8 Hz, 1H), 4.36 (q, J=8.3 Hz, 2H), 3.99 (t, J=7.4 Hz, 2H), 2.98 (br s, 4H), 2.58 (br s, 4H), 2.47 (t, J=7.2 Hz, 2H), 2.13 (s, 3H), 1.89 (s, 3H), 1.84 (p, J=7.3 Hz, 2H).

¹³C NMR (126 MHz, CDCl₃) δ 164.08, 158.47 (d, J=242.0 Hz), 153.08, 150.82 (d, J=9.8 Hz), 144.76, 139.17, 123.46 (q, J=277.8 Hz), 119.43 (d, J=9.4 Hz), 109.74 (d, J=21.4 Hz), 106.55, 104.56 (d, J=25.9 Hz), 66.74 (q, J=35.3 Hz), 55.98, 53.36, 50.98, 39.31, 24.83, 16.69, 10.58.

### Synthesis of Compound 3

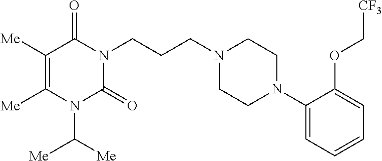 [SMILES: Cc1c(C)n(C(C)C)c(=O)n(CCCN2CCN(c3ccccc3OCC(F)(F)F)CC2)c1=O]

5,6-Dimethyl-3-[3-[4-[2-(2,2,2-trifluoroethoxy)phenyl]-1-piperazinyl]propyl]-2,4(1H,3H)-pyrimidinedione (305.4 mg, 0.693 mmol) and cesium carbonate (451.8 mg, 1.39 mmol) were combined in a 10 mL RBF with a magnetic stir bar and purged under argon. Dimethylformamide (2.3 mL) was added and the suspension stirred for 5 minutes. 2-Iodopropane (90.0 μL, 0.901 mmol) was added in one portion. The reaction was heated to 60° C. and stirred for 18 hours. The reaction was then charged with an additional portion of isopropyl iodide (90.0 μL, 0.901 mmol) and cesium carbonate (451.8 mg, 1.39 mmol) at 10:40 and stirred at 60° C. for four more hours.

The reaction mixture was poured into 50 mL of water. The mixture was extracted with 3×50 mL portions of 1:1 ethyl acetate/hexanes. The combined organic layers were washed with 20 mL of brine, dried with MgSO₄, filtered, and concentrated under reduced pressure.

The residue was purified with 20:80:1 to 50:50:1 ethyl acetate/hexanes/triethylamine and a 25 g silica gel cartridge. The product was obtained as a clear oil (54.6 mg, 0.113 mmol, 16% yield).

Rf=0.2 in 100:1 EtOAc/Et3N

¹H NMR (500 MHz, CDCl₃) δ 7.08-6.98 (m, 1H), 6.97-6.90 (m, 2H), 6.88-6.85 (m, 1H), 4.41 (br s, 1H, overlaps with trifluoroethyoxy quartet at 4.37 ppm), 4.37 (q, J=8.4 Hz, 2H), 3.99 (t, J=7.4 Hz, 2H), 3.06 (br s, 4H), 2.60 (br s, 4H), 2.48 (t, J=7.4 Hz, 2H), 2.22 (s, 3H), 1.96 (s, 3H), 1.85 (p, J=7.3 Hz, 2H), 1.52 (d, J=6.8 Hz, 6H).

¹³C NMR (126 MHz, CDCl₃) δ 163.22, 150.94, 150.23, 146.59, 142.99, 124.12, 123.70 (q, J=277.8 Hz), 122.72, 119.00, 116.77, 107.40, 66.78 (q, J=34.8 Hz), 56.21, 53.45, 50.98, 50.64, 39.81, 24.89, 20.16, 16.96, 12.12.

¹⁹F NMR (470 MHz, CDCl₃) δ −73.96 (t, J=8.2 Hz). (unreferenced, 1H coupled)

### Synthesis of Compound 4

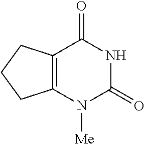 [SMILES: Cn1c2c(c(=O)[nH]c1=O)CCC2]

An oven dried 25 mL flask fitted with a magnetic stir bar and west condenser purged under argon was charged with 6,7-dihydro-1H-cyclopenta[d]pyrimidine-2,4(3H,5H)-dione (501.6 mg, 3.30 mmol), acetonitrile (8.2 mL), and N,O-Bis(trimethylsilyl)acetamide (2.01 mL, 8.24 mmol). The suspension was stirred at room temperature for 3 hours. Methyl iodide (226 μL, 3.63 mmol) was added to the reaction. The reaction was refluxed for 20 hours. An additional portion of methyl iodide (82.1 μL, 1.32 mmol) and the reaction was refluxed for two hours.

The reaction mixture was poured into 30 mL of water and stirred at 0° C. for 15 minutes. The resulting precipitate was collected on filter paper and washed with cold diethyl ether, 20 mL. The solid was dried at 800 mTorr at 100° C. for one hour. The product was obtained as a white powder (365.1 mg, 2.20 mmol, 67% yield).

MP 250.6-251.7° C.

¹H NMR (500 MHz, DMSO) δ 10.99 (s, 1H), 3.20 (s, 3H), 2.86 (t, J=7.7 Hz, 2H), 2.51 (t, J=7.4 Hz, 2H), 1.97 (p, J=7.5 Hz, 2H).

¹³C NMR (126 MHz, DMSO) δ 161.03, 157.82, 152.28, 110.32, 31.71, 31.40, 27.14, 20.61.

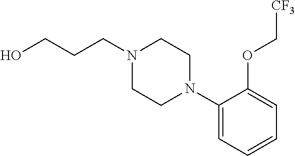 [SMILES: OCCCN1CCN(c2ccccc2OCC(F)(F)F)CC1]

1-[2-(2,2,2-Trifluoroethoxy)phenyl]piperazine dihydrochloride (1.00 g, 3.00 mmol) was purged under argon in a 50 mL RBF with magnetic stir bar. Dimethylformamide (10.0 mL) and potassium carbonate (829.6 mg, 6.00 mmol) were added, and the resulting suspension stirred for 15 minutes at room temperature. 2-(3-bromopropoxy)tetrahydro-2H-pyran (737.3 mg, 3.30 mmol) was added in one portion. The reaction stirred at room temperature for one day. Di-tert-butyl decarbonate (196.5 mg, 0.900 mmol) was added and the reaction stirred for 30 minutes at room temperature.

The reaction mixture was poured into 190 mL of water and extracted with 2×60 mL portions of 1:1 ethyl acetate/hexanes. The combined organic layers were sequentially extracted with 20 mL 1 M HCl and 10 mL of water and then discarded. The aqueous extracts were incubated for 30 minutes and were then basified with 10 mL of 2 M NaOH to give a milky white emulsion. The emulsion was extracted with 2×40 mL of dichloromethane. The combined DCM layers were washed with 10 mL of brine, dried with MgSO4, filtered, and concentrated under reduced pressure.

The crude product was recrystallized from 1.2 mL of hot acetonitrile. The crystals were washed with 1 mL each diethyl ether and hexanes and dried under reduced pressure. The product was obtained as white crystals (501.5 mg, 1.58 mmol, 53% yield).

MP 96.8-98.1° C.

¹H NMR (500 MHz, DMSO) δ 7.11-6.86 (m, 4H), 4.68 (q, J=8.8 Hz, 2H), 4.49 (br s, 1H), 3.47 (t, J=6.3 Hz, 2H), 2.97 (br s, 4H), 2.51 (br s, 4H), 2.39 (t, J=7.3 Hz, 2H), 1.62 (p, J=6.8 Hz, 2H).

¹³C NMR (126 MHz, DMSO) δ 149.57, 141.97, 124.14 (q, J=278.1 Hz), 123.08, 122.31, 118.59, 115.11, 65.24 (q, J=33.9 Hz), 59.46, 55.21, 53.10, 50.15, 29.58.

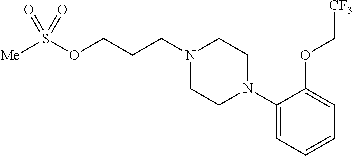 [SMILES: CS(=O)(=O)OCCCN1CCN(c2ccccc2OCC(F)(F)F)CC1]

4-[2-(2,2,2-Trifluoroethoxy)phenyl]-1-piperazinepropanol was purged under argon in an oven dried 10 mL round bottom flask fitted with a rubber septum. The substrate was dissolved in dichloromethane, and the solution was cooled to 0° C. Triethylamine was added in one portion and then the methanesulfonyl chloride was added dropwise. The reaction stirred at 0° C. for 1 hour.

Saturated sodium carbonate, 2.5 mL, was added and the mixture stirred for 5 minutes. The organic layer was separated, and the aqueous layer was extracted with an additional 2×2 mL portions of dichloromethane. The combined DCM layers were dried with MgSO4, filtered, and concentrated under reduced pressure. Obtained 651.3 mg (1.64 mmol, assumed quantitative yield) of crude product. An aliquot was taken for NMR and the remainder was dissolved in 1,4-dioxane to give a 25 wt % solution.

¹H NMR (500 MHz, CDCl₃) δ 7.10-7.01 (m, 1H), 7.01-6.93 (m, 2H), 6.92-6.84 (m, 1H), 4.39 (q, J=8.4 Hz, 2H), 4.33 (t, J=6.3 Hz, 2H), 3.09 (br s, 4H), 3.02 (s, 3H), 2.62 (br s, 4H), 2.53 (t, J=7.0 Hz, 2H), 1.97 (p, J=6.6 Hz, 2H).

¹³C NMR (126 MHz, CDCl₃) δ 150.14, 142.65, 124.01, 123.61 (q, J=278.1 Hz), 122.86, 118.96, 116.40, 68.42, 66.68 (q, J=35.1 Hz), 54.00, 53.44, 50.53, 37.28, 26.46.

¹⁹F NMR (470 MHz, CDCl₃) δ −73.95 (t, J=8.3 Hz). (unreferenced, 1H coupled)

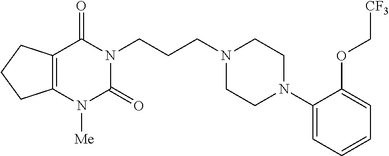 [SMILES: Cn1c2c(c(=O)n(CCCN3CCN(c4ccccc4OCC(F)(F)F)CC3)c1=O)CCC2]

6,7-dihydro-1-methyl-1H-Cyclopentapyrimidine-2,4(3H,5H)-dione (75.5 mg, 0.454 mmol), cesium carbonate (296.1 mg, 0.909 mmol), and dimethylformamide (1.5 mL) were combined in an oven-dried 10 mL RBF with magnetic stir bar. The suspension stirred at room temperature for 15 minutes. A 25 wt. % solution of mesylate in 1,4-dioxane (937 mg, 0.591 mmol) was added and the reaction was heated to 60° C. for 2 hours.

The reaction mixture was poured into 30 mL of water and extracted with 3×30 mL portions of 1:1 ethyl acetate/hexanes. The combined organic layers were washed with 10 mL of brine, dried with MgSO₄, filtered, and concentrated under reduced pressure.

The crude product was purified with 10:90:1 to 50:50:1 ethyl acetate/hexanes/triethylamine and a 25 g silica gel cartridge. The product was repurified by prep-TLC with 5% methanol in dichloromethane on a 20 cm×20 cm×1000 μm silica plate. The product was obtained as a clear oil (109.2 mg, 0.240 mmol, 53% yield).

¹H NMR (500 MHz, CDCl₃) δ 7.05-6.95 (m, 1H), 6.94-6.89 (m, 2H), 6.88-6.81 (m, 1H), 4.35 (q, J=8.4 Hz, 2H), 3.99 (t, J=7.4 Hz, 2H), 3.31 (s, 3H), 3.04 (br s, 4H), 2.81 (t, J=1.7 Hz, 2H), 2.71 (t, J=7.5 Hz, 2H), 2.58 (br s, 4H), 2.47 (t, J=7.2 Hz, 2H), 2.05 (p, J=7.5 Hz, 2H), 1.83 (p, 2H).

¹³C NMR (126 MHz, CDCl₃) δ 161.01, 154.74, 152.84, 150.15, 142.92, 124.04, 123.65 (q, J=278.2 Hz), 122.64, 118.88, 116.68, 111.56, 66.68 (q, J=35.0 Hz), 55.97, 53.34, 50.60, 39.89, 32.82, 32.43, 27.91, 24.81, 20.96.

¹⁹F NMR (470 MHz, CDCl₃) δ −73.96. (unreferenced, proton decoupled).

### Synthesis of Compound 5

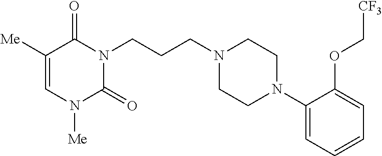 [SMILES: Cc1cn(C)c(=O)n(CCCN2CCN(c3ccccc3OCC(F)(F)F)CC2)c1=O]

RS-100329 (179.1 mg, 0.420 mmol) and cesium carbonate (410.5 mg, 1.26 mmol) were combined in a 25 mL RBF with a magnetic stir bar and purged under argon. Dimethylformamide (4.2 mL) was added and the suspension stirred for 5 minutes. Methyl iodide (28.8 μL, 0.462 mmol) was added in one portion and the reaction stirred at room temperature for 3 hours.

The reaction mixture was poured into 60 mL of water. The mixture was extracted with 5×40 mL portions of 3:1 ethyl acetate/hexanes. The combined organic layers were dried with MgSO₄, filtered, and concentrated under reduced pressure.

The residue was purified with 50:50:1 to 100:0:1 ethyl acetate/hexanes/triethylamine and a 12 g silica gel cartridge. The product was obtained as a viscous yellow oil (165.8 mg, 0.376 mmol, 90% yield).

¹H NMR (500 MHz, CDCl₃) δ 6.99-6.93 (m, 1H), 6.92 (s, 1H), 6.89-6.84 (m, 2H), 6.83-6.78 (m, 1H), 4.32 (q, J=8.4 Hz, 2H), 3.97 (t, J=7.4 Hz, 2H), 3.27 (s, 3H), 3.00 (br s, 4H), 2.54 (br s, 4H), 2.43 (t, J=7.2 Hz, 2H), 1.84 (s, 3H), 1.80 (p, J=7.2 Hz, 2H).

¹³C NMR (126 MHz, CDCl₃) δ 163.80, 151.61, 150.02, 142.76, 139.09, 123.92, 123.56 (q, J=278.3 Hz), 122.53, 118.78, 116.57, 109.46, 66.55 (q, J=34.9 Hz), 55.84, 53.20, 50.43, 39.81, 36.43, 24.52, 12.83.

### Synthesis of Compound 7

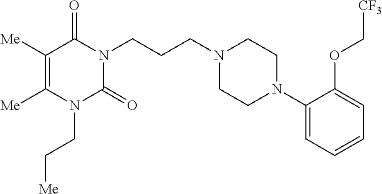 [SMILES: CCCn1c(C)c(C)c(=O)n(CCCN2CCN(c3ccccc3OCC(F)(F)F)CC2)c1=O]

5,6-Dimethyl-3-[3-[4-[2-(2,2,2-trifluoroethoxy)phenyl]-1-piperazinyl]propyl]-2,4(1H,3H)-pyrimidinedione (205.9 mg, 0.468 mmol) and cesium carbonate (304.6 mg, 0.935 mmol) were combined in a 10 mL RBF with magnetic stir bar. The flask was purged under argon for one minute then DMF (1.6 mL) was added. The suspension stirred for 10 minutes at room temperature. 1-Iodopropane was added in one portion and the reaction continued to stir at room temperature for 4 hours.

The reaction mixture was poured into 30 mL of water. The mixture was extracted with 3×30 mL portions of 1:1 ethyl acetate/hexanes. The combined organic layers were dried with MgSO₄, filtered, and concentrated under reduced pressure.

The residue was purified with 20:80:1 to 45:55:1 ethyl acetate/hexanes/triethylamine and a 12 g silica gel cartridge. The product was collected as a white solid (154.4 mg, 0.320 mmol, 68% yield)

Rf=0.175, 80:20:1 Ethyl acetate/hexanes/triethylamine

MP 104.3-106.1° C.

¹H NMR (500 MHz, CDCl₃) δ 6.99-6.92 (m, 1H), 6.91-6.84 (m, 2H), 6.83-6.76 (m, 1H), 4.32 (q, J=8.4 Hz, 2H), 3.97 (t, J=7.4 Hz, 2H), 3.74 (t, J=7.9 Hz, 2H), 3.00 (br s, 4H), 2.54 (br s, 4H), 2.43 (t, J=7.2 Hz, 2H), 2.17 (s, 3H), 1.91 (s, 3H), 1.80 (p, J=7.3 Hz, 2H), 1.60 (h, J=7.5 Hz, 2H), 0.89 (t, J=7.4 Hz, 3H).

¹³C NMR (126 MHz, CDCl₃) δ 163.12, 151.68, 150.25, 146.31, 143.03, 124.13, 123.72 (q, J=278.3 Hz), 122.72, 118.99, 116.80, 107.34, 66.79 (q, J=35.1 Hz), 56.17, 53.45, 50.68, 46.91, 40.30, 24.83, 22.46, 16.14, 11.87, 11.17.

¹⁹F NMR (470 MHz, CDCl₃) δ −73.96 (t, J=8.3 Hz). (unreferenced, 1H coupled)

### Synthesis of Compound 8

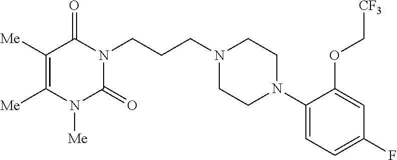 [SMILES: Cc1c(C)n(C)c(=O)n(CCCN2CCN(c3ccc(F)cc3OCC(F)(F)F)CC2)c1=O]

5,6-Dimethyl-3-[3-[4-[2-(2,2,2-trifluoroethoxy)-4-fluorophenyl]-1-piperazinyl]propyl]-2,4(1H,3H)-pyrimidinedione (196.6 mg, 0.429 mmol) and cesium carbonate (279.4 mg, 0.858 mmol) were combined in a 10 mL RBF with magnetic stir bar. The flask was purged under argon then DMF (2.9 mL) was added. The suspension stirred for 20 minutes at room temperature. Iodomethane (29.4 μL, 0.472 mmol) was added in one portion and the reaction continued to stir at room temperature for 1 hour.

The reaction mixture was poured into 30 mL of water. The mixture was extracted with 3×30 mL portions of 1:1 ethyl acetate/hexanes. The combined organic layers were washed with brine, dried with MgSO₄, filtered, and concentrated under reduced pressure.

The residue was purified with 30:70:1 to 75:25:1 ethyl acetate/hexanes/triethylamine and a 12 g silica gel cartridge. The product was collected as an off white solid (181.5 mg, 0.384 mmol, 90% yield).

Rf=0.14 in ethyl acetate/triethylamine 100:1

MP 149.6-151.7° C.

¹H NMR (500 MHz, CDCl₃) δ 6.75 (dd, J=8.9, 5.9 Hz, 1H), 6.59 (ddd, J=8.7, 7.9, 2.8 Hz, 1H), 6.52 (dd, J=9.5, 2.8 Hz, 1H), 4.27 (q, J=8.4 Hz, 2H), 3.91 (t, J=7.5 Hz, 2H), 3.29 (s, 3H), 2.87 (br s, 4H), 2.47 (br s, 4H), 2.36 (t, J=7.2 Hz, 2H), 2.11 (s, 3H), 1.85 (s, 3H), 1.73 (p, J=7.3 Hz, 2H).

¹³C NMR (126 MHz, CDCl₃) δ 162.93, 158.25 (d, J=241.9 Hz), 151.80, 150.65, 146.63, 139.15 (d, J=3.1 Hz), 123.35 (q, J=278.1 Hz), 119.30 (d, J=9.3 Hz), 109.58 (d, J=21.3 Hz), 107.03, 104.54 (d, J=25.5 Hz), 66.61 (q, J=35.4 Hz), 55.85, 53.19, 50.86, 40.19, 31.93, 24.61, 16.51, 11.60.

HRMS (ESI-TOF) m/z: [M+H]+ calcd for C₂₂H₂₉F₄N₄O₃, 473.2176; found, 473.2170.

### Synthesis of Compound 9

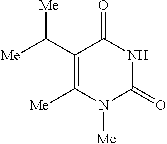 [SMILES: Cc1c(C(C)C)c(=O)[nH]c(=O)n1C]

An oven dried 10 mL flask fitted with a magnetic stir bar and west condenser purged under argon was charged with 5-isopropyl-6-methyluracil (184.1 mg, 1.09 mmol), acetonitrile (2.7 mL), and N,O-Bis(trimethylsilyl)acetamide (669 μL, 2.74 mmol). The suspension was stirred at room temperature for 3 hours. Methyl iodide (102 μL, 1.64 mmol) was added to the reaction. The reaction was refluxed for 15 hours. An additional portion of methyl iodide (102 μL, 1.64 mmol) and the reaction was refluxed for 4 hours.

The reaction mixture was poured into 50 mL of water and extracted with 3×25 mL portions of dichloromethane. The combined organic layers were dried with MgSO4, filtered, and concentrated under reduced pressure with 500 mg of silica gel.

The adsorbed residue was purified using a 12 g silica gel cartridge and 5 to 70% ethyl acetate in hexanes. The product was obtained as a white solid (168.1 mg, 0.922 mmol, 84% yield).

Rf=0.14 in 1:1 ethyl acetate/hexanes

¹H NMR (500 MHz, CDCl₃) δ 9.49 (s, 1H), 3.38 (s, 3H), 3.03 (hept, J=7.1 Hz, 1H), 2.26 (s, 3H), 1.25 (d, J=7.1 Hz, 6H).

¹³C NMR (126 MHz, CDCl₃) δ 162.63, 151.65, 148.49, 117.40, 31.39, 27.51, 20.54, 16.54.

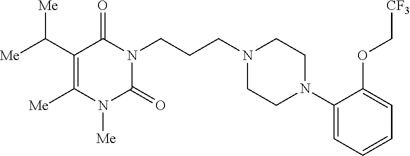 [SMILES: Cc1c(C(C)C)c(=O)n(CCCN2CCN(c3ccccc3OCC(F)(F)F)CC2)c(=O)n1C]

1-methyl-5-isopropyl-6-methyluracil (63.9 mg, 0.351 mmol), cesium carbonate (228.5 mg, 0.701 mmol), and dimethylformamide (1.2 mL) were combined in an oven-dried 10 mL RBF with magnetic stir bar. The suspension stirred at 70° C. for 15 minutes. A 25 wt. % solution of mesylate in 1,4-dioxane (723 mg, 0.456 mmol) was added and the reaction was stirred at 70° C. for two hours.

The reaction mixture was poured into 30 mL of water and extracted with 3×30 mL portions of 1:1 ethyl acetate/hexanes. The combined organic layers were washed with 10 mL of brine, dried with MgSO₄, filtered, and concentrated under reduced pressure.

The crude product was purified with 10:90:1 to 60:40:1 ethyl acetate/hexanes/triethylamine and a 25 g silica gel cartridge. The product was obtained as a pale yellow oil (138.8 mg, 0.288 mmol, 82% yield).

Rf=0.12 in 50:50:1 hexanes/ethyl acetate/triethylamine on TLC

¹H NMR (500 MHz, CDCl₃) δ 7.02 (ddd, J=8.4, 7.2, 1.6 Hz, 1H), 6.98-6.90 (m, 2H), 6.88 (dd, J=8.3, 1.6 Hz, 1H), 4.38 (q, J=8.4 Hz, 2H), 4.01 (t, J=7.3 Hz, 2H), 3.41 (s, 3H), 3.17-2.87 (m, 5H, overlapping isopropyl methine and piperazine methylene resonances), 2.60 (br s, 4H), 2.49 (t, J=7.3 Hz, 2H), 2.25 (s, 3H), 1.86 (p, 2H), 1.27 (d, J=7.0 Hz, 6H).

¹³C NMR (126 MHz, CDCl₃) δ 161.93, 152.04, 150.28, 146.29, 143.04, 124.15, 123.74 (q, J=278.3 Hz), 122.75, 119.01, 116.82, 116.63, 66.82 (q, J=35.0 Hz), 56.24, 53.48, 50.72, 40.11, 32.16, 27.74, 24.89, 20.66, 16.47.

¹⁹F NMR (470 MHz, CDCl₃) δ −73.96 (t, J=8.2 Hz). (unreferenced, proton coupled)

### Synthesis of Compound 10

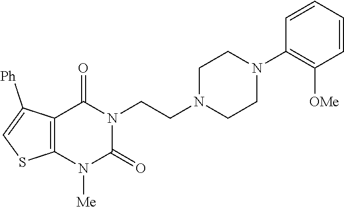 [SMILES: COc1ccccc1N1CCN(CCn2c(=O)c3c(-c4ccccc4)csc3n(C)c2=O)CC1]

A-119637 formate (154.5 mg, 0.304 mmol) and cesium carbonate (198.0 mg, 6.08 mmol) were combined in a 10 mL RBF with a magnetic stir bar and purged under argon. Dimethylformamide (1.0 mL) was added and the suspension stirred for 5 minutes. Methyl iodide (20.8 μL, 0.334 mmol) was added in one portion and the reaction stirred at room temperature for 1 hour.

The reaction mixture was poured into 25 mL of water. The mixture was extracted with 3×30 mL portions of 1:1 ethyl acetate/hexanes. The combined organic layers were dried with MgSO₄, filtered, and concentrated under reduced pressure with 500 mg silica gel.

The adsorbed residue was purified with 20:80:1 to 60:40:1 ethyl acetate/hexanes/triethylamine and a 12 g silica gel cartridge. The product was obtained as a viscous yellow oil (103.2 mg, 0.217 mmol, 71% yield).

¹H NMR (500 MHz, CDCl₃) δ 7.52-7.46 (m, 2H), 7.43-7.32 (m, 3H), 6.96 (ddd, J=7.9, 6.8, 2.2 Hz, 1H), 6.94-6.86 (m, 2H), 6.83 (dd, J=8.0, 1.4 Hz, 1H), 6.69 (s, 1H), 4.20 (t, J=7.1 Hz, 2H), 3.83 (s, 3H), 3.56 (s, 3H), 3.06 (br s, 4H), 2.76 (br s, 4H), 2.69 (t, J=7.1 Hz, 2H).

¹³C NMR (126 MHz, CDCl₃) δ 157.87, 154.88, 152.24, 150.69, 141.95, 141.45, 134.99, 129.25, 128.02, 127.78, 122.73, 120.94, 118.13, 113.23, 112.36, 111.17, 55.53, 55.32, 53.48, 50.66, 38.76, 34.28.

### Synthesis of Compound 11

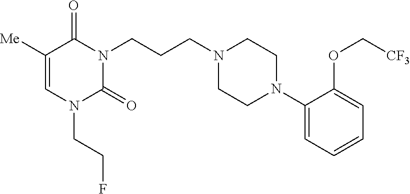 [SMILES: Cc1cn(CCF)c(=O)n(CCCN2CCN(c3ccccc3OCC(F)(F)F)CC2)c1=O]

RS-100329 (201.7 mg, 0.473 mmol) and cesium carbonate (462.3 mg, 1.42 mmol) were combined in a 25 mL RBF with a magnetic stir bar and purged under argon. Dimethylformamide (4.7 mL) was added and the suspension stirred for 5 minutes. 2-fluoroethyl nosylate (123.8 mg, 0.497 mmol) was added in one portion and the reaction stirred at room temperature for 3 hours.

The reaction mixture was poured into 60 mL of water. The mixture was extracted with 5×40 mL portions of 3:1 ethyl acetate/hexanes. The combined organic layers were dried with MgSO₄, filtered, and concentrated under reduced pressure.

The residue was purified with 30:70:1 to 70:30:1 ethyl acetate/hexanes/triethylamine and a 12 g silica gel cartridge. The product was obtained as a viscous yellow oil (190.2 mg, 0.403 mmol, 85% yield).

Rf=0.30 ethyl acetate/triethylamine 100:1

¹H NMR (500 MHz, CDCl₃) δ 7.05-6.96 (m, 2H), 6.96-6.88 (m, 2H), 6.89-6.82 (m, 1H), 4.62 (dt, J=47.3, 4.5 Hz, 2H), 4.36 (q, J=8.4 Hz, 2H), 4.04-3.93 (m, 4H), 3.05 (br s, 4H), 2.59 (br s, 4H), 2.48 (t, J=7.2 Hz, 2H), 1.89 (d, J=1.2 Hz, 3H), 1.85 (p, J=7.2 Hz, 2H).

¹³C NMR (126 MHz, CDCl₃) δ 163.76, 151.40, 150.15, 142.84, 138.98, 124.03, 123.67 (q, J=278.4 Hz), 122.70, 118.94, 116.59, 109.86, 81.72 (d, J=169.5 Hz), 66.68 (q, J=35.0 Hz), 55.98, 53.34, 50.51, 49.75 (d, J=19.3 Hz), 40.01, 24.63, 13.01.

¹⁹F NMR (470 MHz, CDCl₃) δ −73.96 (t, J=8.5 Hz), −224.09 (tt, J=47.7, 27.5 Hz). (unreferenced, 1H coupled)

### Synthesis of Compound 12

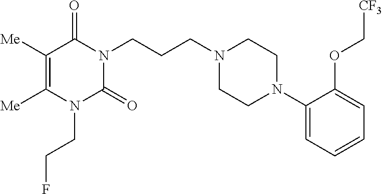 [SMILES: Cc1c(C)n(CCF)c(=O)n(CCCN2CCN(c3ccccc3OCC(F)(F)F)CC2)c1=O]

5,6-Dimethyl-3-[3-[4-[2-(2,2,2-trifluoroethoxy)phenyl]-1-piperazinyl]propyl]-2,4(1H,3H)-pyrimidinedione (100.0 mg, 0.227 mmol) and cesium carbonate (221.9 mg, 0.681 mmol) were combined in a 10 mL RBF with a magnetic stir bar and purged under argon. Dimethylformamide (2.3 mL) was added. 2-Fluoroethyl nosylate (59.4 mg, 0.238 mmol) was added in one portion and the reaction stirred at room temperature for 9 hours.

The reaction mixture was poured into 40 mL of water. The mixture was extracted with 3×40 mL portions of 1:1 ethyl acetate/hexanes. The combined organic layers were dried with MgSO₄, filtered, and concentrated under reduced pressure with 500 mg of silica gel.

The adsorbed residue was purified with 50:50 to 100:0 ethyl acetate/hexanes and a 12 g silica gel cartridge. The product was obtained as a clear oil (5.04 mg, 0.113 mmol, 50% yield).

¹H NMR (500 MHz, CDCl₃) δ 7.05-6.98 (m, 1H), 6.97-6.92 (m, 2H), 6.90-6.76 (m, 1H), 4.67 (dt, J=47.9, 4.5 Hz, 2H), 4.42-4.33 (m, 2H), 4.17 (dt, J=25.7, 4.5 Hz, 2H), 4.03 (t, J=7.3 Hz, 2H), 3.06 (br s, 4H), 2.60 (br s, 4H), 2.49 (t, J=7.2 Hz, 2H), 2.26 (s, 3H), 1.98 (s, 3H), 1.86 (p, J=7.2 Hz, 2H).

¹³C NMR (126 MHz, CDCl₃) δ 163.05, 151.72, 150.27, 146.71, 143.01, 124.14, 123.73 (q, J=278.0 Hz), 122.75, 119.00, 116.80, 107.79, 82.01 (d, J=169.3 Hz), 66.81 (q, J=35.0 Hz), 56.11, 53.44, 50.66, 45.83 (d, J=19.8 Hz), 40.34, 24.77, 16.74 (d, J=4.8 Hz), 12.02.

HRMS (ESI-TOF) m/z: [M+H]+ calcd for C₂₃H₃₁F₄N₄O₃, 487.2332; found, 487.2306.

### Synthesis of Compound 13

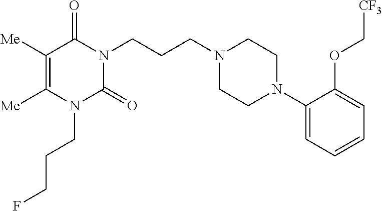 [SMILES: Cc1c(C)n(CCCF)c(=O)n(CCCN2CCN(c3ccccc3OCC(F)(F)F)CC2)c1=O]

5,6-Dimethyl-3-[3-[4-[2-(2,2,2-trifluoroethoxy)phenyl]-1-piperazinyl]propyl]-2,4(1H,3H)-pyrimidinedione (215.1 mg, 0.488 mmol) and cesium carbonate (318.2 mg, 0.977 mmol) were combined in a 10 mL RBF with magnetic stir bar. The flask was purged under argon for one minute then DMF (1.6 mL) was added. The suspension stirred for 10 minutes at room temperature. 1-iodo-3-fluoropropane was added in one portion and the reaction stirred at room temperature for 2.5 hours.

The reaction mixture was poured into 30 mL of water. The mixture was extracted with 3×30 mL portions of 1:1 ethyl acetate/hexanes. The combined organic layers were dried with MgSO₄, filtered, and concentrated under reduced pressure.

The residue was purified with 15:85:1 to 40:60:1 ethyl acetate/hexanes/triethylamine and a 12 g silica gel cartridge. The product was obtained as a white solid (179.8 mg, 0.359 mmol, 74% yield).

Rf=0.15, 80:20:1 Ethyl acetate/hexanes/triethylamine

MP 94.5-96.3° C.

¹H NMR (500 MHz, CDCl₃) δ 7.13-6.97 (m, 1H), 6.97-6.91 (m, 2H), 6.90-6.83 (m, 1H), 4.51 (dt, J=48.0, 5.1 Hz, 2H), 4.37 (q, J=8.4 Hz, 2H), 4.01 (t, J=7.3 Hz, 3H), 3.06 (br s, 4H), 2.59 (br s, 4H), 2.48 (t, J=7.2 Hz, 2H), 2.25 (s, 3H), 2.05 (dp, J=27.4, 6.2 Hz, 2H), 1.97 (s, 3H), 1.85 (p, J=7.4 Hz, 2H).

¹³C NMR (126 MHz, CDCl₃) δ 163.02, 151.68, 150.22, 146.20, 142.97, 124.10, 123.70 (q, J=278.2 Hz), 122.71, 118.96, 116.73, 107.64, 81.47 (d, J=165.0 Hz), 66.75 (q, J=35.1 Hz), 56.11, 53.43, 50.66, 42.16 (d, J=4.1 Hz), 40.29, 29.93 (d, J=19.7 Hz), 24.80, 16.07, 11.90.

¹⁹F NMR (470 MHz, CDCl₃) δ −73.96 (t, J=8.4 Hz), −220.34 (tt, J=47.3, 28.2 Hz). (unreferenced, 1H coupled)

### Synthesis of Compound 14

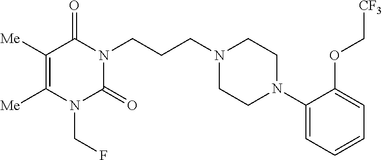 [SMILES: Cc1c(C)n(CF)c(=O)n(CCCN2CCN(c3ccccc3OCC(F)(F)F)CC2)c1=O]

5,6-Dimethyl-3-[3-[4-[2-(2,2,2-trifluoroethoxy)phenyl]-1-piperazinyl]propyl]-2,4(1H,3H)-pyrimidinedione (149.7 mg, 0.3399 mmol) and cesium carbonate (221.5 mg, 0.680 mmol) were combined in a 10 mL RBF with a magnetic stir bar and purged under argon. 660 μL of a 94.6 mg/mL fluoroiodomethane in dimethylformamide solution was added, and the reaction stirred at room temperature for 1 hour.

The reaction mixture was poured into 40 mL of water. The mixture was extracted with 3×40 mL portions of 1:1 ethyl acetate/hexanes. The combined organic layers were washed with 10 mL brine, dried with MgSO₄, filtered, and concentrated under reduced pressure with 500 mg of silica gel.

The adsorbed residue was purified with 25:75 to 75:25 ethyl acetate/hexanes and a 12 g silica gel cartridge. The product was obtained as a clear oil (113.7 mg, 0.241 mmol, 71% yield).

Rf=0.24 in ethyl acetate

¹H NMR (500 MHz, CDCl₃) δ 7.04-6.96 (m, 1H), 6.94-6.88 (m, 2H), 6.88-6.82 (m, 1H), 5.94 (d, J=52.1 Hz, 2H), 4.35 (q, J=8.4 Hz, 2H), 4.01 (t, J=7.4 Hz, 2H), 3.03 (br s, 4H), 2.57 (br s, 4H), 2.47 (t, J=7.1 Hz, 2H), 2.28 (s, 3H), 1.96 (s, 3H), 1.84 (p, J=7.2 Hz, 2H).

¹³C NMR (126 MHz, CDCl₃) δ 162.71, 151.36, 150.14, 144.60, 142.87, 124.76, 124.02, 122.65, 118.86, 116.65, 109.10, 82.18 (d, J=201.3 Hz), 66.67 (q, J=34.4 Hz), 55.92, 53.32, 50.52, 40.43, 24.45, 15.20, 11.58.

HRMS (ESI-TOF) m/z: [M+H]⁺ calcd for C₂₂H₂₉F₄N₄O₃, 473.2176; found, 473.2172.

### Synthesis of Compound 15

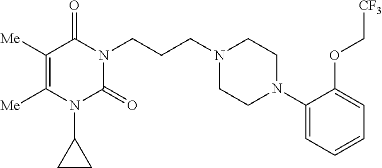 [SMILES: Cc1c(C)n(C2CC2)c(=O)n(CCCN2CCN(c3ccccc3OCC(F)(F)F)CC2)c1=O]

5,6-Dimethyl-3-[3-[4-[2-(2,2,2-trifluoroethoxy)phenyl]-1-piperazinyl]propyl]-2,4(1H,3H)-pyrimidinedione (168.7 mg, 0.383 mmol), cyclopropyl boronic acid (65.8 mg, 0.766 mmol), copper(II) acetate (69.6 mg, 0.383 mmol), 2,2′-bipyridine (47.9 mg, 0.306 mmol), and acetonitrile (3.83 mL) were combined in a 15 mL RBF with magnetic stir bar. Sodium tert-butoxide (2.0 M in tetrahydrofuran, 383 μL, 0.766 mmol) was added in one portion. The reaction vessel was fitted with a condenser and heated to 70° C. for 18 hours, open to air.

The reaction mixture was concentrated in vacuo with 1 g of silica gel. The adsorbed residue was purified with a 25 g silica gel cartridge and 10:90:1 to 60:40:1 ethyl acetate/hexanes/triethylamine. The product was obtained as a pale orange residue (132.4 mg, 0.276 mmol, 72% yield).

¹H NMR (500 MHz, CDCl₃) δ 7.04-6.97 (m, 1H), 6.96-6.89 (m, 2H), 6.88-6.84 (m, 1H), 4.36 (q, J=8.4 Hz, 2H), 3.98 (t, J=7.5 Hz, 2H), 3.05 (br s, 4H), 2.74 (tt, J=6.9, 4.0 Hz, 1H), 2.58 (br s, 4H), 2.47 (t, J=7.2 Hz, 2H), 2.34 (s, 2H), 1.92 (s, 3H), 1.83 (p, J=7.3 Hz, 2H), 1.21-1.12 (m, 2H), 0.84-0.76 (m, 2H).

¹³C NMR (126 MHz, CDCl₃) δ 163.22, 152.33, 150.20, 148.42, 142.97, 124.09, 123.68 (q, J=277.1 Hz), 122.68, 118.94, 116.74, 107.66, 66.74 (q, J=35.1 Hz), 56.13, 53.40, 50.64, 40.14, 28.40, 24.82, 16.92, 11.60, 10.70.

¹⁹F NMR (470 MHz, CDCl₃) δ −73.96. (unreferenced, 1H decoupled)

### Synthesis of Compound 16

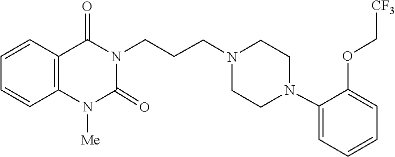 [SMILES: Cn1c(=O)n(CCCN2CCN(c3ccccc3OCC(F)(F)F)CC2)c(=O)c2ccccc21]

3-[3-[4-[2-(2,2,2-trifluoroethoxy)phenyl]-1-piperazinyl]propyl]-2,4(1H,3H)-Quinazolinedione formate (100.0 mg, 0.197 mmol) and cesium carbonate (128.1 mg, 0.393 mmol) were combined in a 10 mL RBF with a magnetic stir bar and purged under argon. Dimethylformamide (1.0 mL) was added, and the suspension was briefly heated with a heat gun and then cooled to room temperature. Methyl iodide (13.5 μL, 0.216 mmol) was added in one portion and the reaction stirred at room temperature for 90 minutes.

The reaction mixture was poured into 20 mL of water. The mixture was extracted with 3×20 mL portions of 3:2 ethyl acetate/hexanes. The combined organic layers were washed with 10 mL of brine, dried with MgSO₄, filtered, and concentrated under reduced pressure with 500 mg of silica gel.

The adsorbed residue was purified with 30:70:1 to 60:40:1 ethyl acetate/hexanes/triethylamine and a 12 g silica gel cartridge. The product was obtained as a yellow oil (55.0 mg, 0.115 mmol, 59% yield).

¹H NMR (500 MHz, CDCl₃) δ 8.22 (dd, J=7.9, 1.7 Hz, 1H), 7.66 (ddd, J=8.6, 7.2, 1.6 Hz, 1H), 7.25 (td, J=8.0, 1.3 Hz, 1H), 7.19 (d, J=8.4 Hz, 1H), 7.02 (td, J=7.6, 1.6 Hz, 1H), 6.94 (td, J=7.6, 1.6 Hz, 1H), 6.91-6.83 (m, 2H), 4.38 (q, J=8.4 Hz, 2H), 4.19 (t, J=7.3 Hz, 2H), 3.59 (s, 3H), 3.02 (br s, 4H), 2.61 (br s, 4H), 2.55 (t, J=7.1 Hz, 2H), 1.95 (p, J=7.2 Hz, 2H).

¹³C NMR (126 MHz, CDCl₃) δ 161.91, 151.11, 150.25, 142.99, 140.62, 135.07, 128.95, 124.11, 123.715 (q, J=277 Hz), 122.96, 122.73, 118.98, 116.79, 115.73, 113.54, 66.80 (q, J=35.1 Hz), 56.14, 53.45, 50.62, 40.62, 30.76, 24.84.

### Synthesis of Compound 17

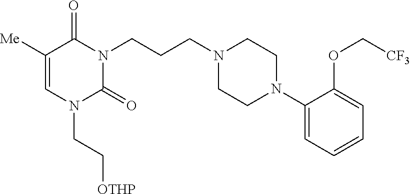

RS-100329 (267.9 mg, 0.628 mmol), cesium carbonate (0.409 mg, 1.26 mmol), and potassium iodide (104.3 mg, 0.628 mmol) were combined in a 25 mL RBF with magnetic stir bar. The flask was purged under argon then DMF (4.2 mL) was added. The suspension stirred for 5 minutes at room temperature. 2-(2-Bromoethoxy)tetrahydro-2H-pyran (123 μL, 0.817 mmol) was added in one portion, and the reaction continued to stir at room temperature for 18 hours.

The reaction mixture was poured into 60 mL of half saturated brine. The mixture was extracted with 3×40 mL 3:1 ethyl acetate/hexanes. The combined organic layers were dried with MgSO₄, filtered, and concentrated under reduced pressure.

The residue was purified with 30:70:1 to 70:30:1 ethyl acetate/hexanes/triethylamine and a 12 g silica gel cartridge. The product was collected as a viscous yellow oil (272.2 mg, 0.491 mmol, 78% yield).

Rf=0.18 100:1 ethyl acetate/triethylamine

¹H NMR (500 MHz, CDCl₃) δ 7.16 (s, 1H), 7.06-6.97 (m, 1H), 6.97-6.83 (m, 3H), 4.60-4.50 (m, 1H), 4.40 (q, J=8.4 Hz, 2H), 4.04 (t, J=7.4 Hz, 2H), 4.00-3.80 (m, 3H), 3.77-3.55 (m, 2H), 3.55-3.37 (m, 1H), 3.07 (br s, 4H), 2.60 (br s, 4H), 2.49 (t, J=7.0 Hz, 2H), 1.92 (s, 3H), 1.87 (p, J=7.5 Hz, 2H), 1.78-1.61 (m, 1H), 1.70-1.61 (m, 1H), 1.61-1.39 (m, 4H).

¹³C NMR (126 MHz, CDCl₃) δ 163.54, 151.08, 149.85, 142.63, 139.61, 123.72, 123.42 (q, J=278.4 Hz), 122.28, 118.56, 116.48, 108.31, 98.57, 66.35 (q, J=35.0 Hz), 64.86, 61.91, 55.67, 53.03, 50.27, 39.56, 30.14, 24.98, 24.41, 19.05, 12.58.

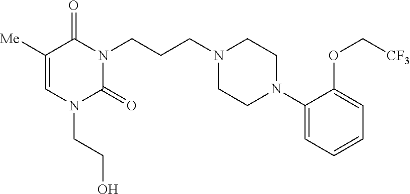 [SMILES: Cc1cn(CCO)c(=O)n(CCCN2CCN(c3ccccc3OCC(F)(F)F)CC2)c1=O]

The above 2-tetrahydropyranyl ether (77.2 mg, 0.139 mmol) was dissolved in methanol (1.0 mL). Hydrogen chloride in isopropanol (41.8 μL, 0.209 mmol) was added in one portion via micropipette. The mixture was incubated for one hour at room temperature.

The reaction mixture was concentrated under reduced pressure. Half saturated sodium bicarbonate (15 mL) was added, and the mixture was extracted with 3×25 mL portions of ethyl acetate. The combined organic layers were dried with MgSO4, filtered, and concentrated under reduced pressure.

The product was recrystallized from hot ethyl acetate (1 mL), washed with cold ethyl acetate and hexanes, dried under reduced pressure to give the product as a white solid (30.5 mg, 0.0648 mmol, 47% yield).

MP 149.2-150.1° C.

Rf=0.13 100:1 ethyl acetate/triethylamine

### Synthesis of Compound 18

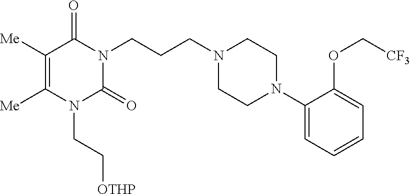

5,6-Dimethyl-3-[3-[4-[2-(2,2,2-trifluoroethoxy)phenyl]-1-piperazinyl]propyl]-2,4(1H,3H)-pyrimidinedione (301.4 mg, 0.684 mmol), cesium carbonate (445.9 mg, 1.37 mmol), and potassium iodide (113.6 mg, 0.684 mmol) were combined in a 25 mL RBF with magnetic stir bar. The flask was purged under argon then DMF (2.3 mL) was added. The suspension stirred for 15 minutes at 60° C. 2-(2-Bromoethoxy)tetrahydro-2H-pyran (134 μL, 0.890 mmol) was added in one portion, and the reaction continued to stir at 60° C. for 3.5 hours.

The reaction mixture was poured into 50 mL of half saturated brine. The mixture was extracted with 3×40 mL portions of 1:1 ethyl acetate/hexanes. The combined organic layers were dried with MgSO₄, filtered, and concentrated under reduced pressure.

The residue was purified with 5:95:1 to 50:50:1 ethyl acetate/hexanes/triethylamine and a 25 g silica gel cartridge. The product was collected as a clear oil (328.2 mg, 0.577 mmol, 84% yield).

Rf=0.21 in 100:1 EtOAc/Et3N

¹H NMR (500 MHz, CDCl₃) δ 7.07-7.00 (m, 1H), 6.98-6.92 (m, 2H), 6.92-6.88 (m, 1H), 4.60-4.51 (m, 1H), 4.39 (q, J=8.4 Hz, 2H), 4.16-4.07 (m, 2H), 4.06-4.03 (m, 2H), 3.97 (ddd, J=10.4, 6.1, 4.4 Hz, 1H), 3.79-3.60 (m, 2H), 3.54-3.35 (m, 1H), 3.08 (br s, 4H), 2.62 (br s, 4H), 2.51 (t, J=7.3 Hz, 2H), 2.34 (s, 3H), 2.00 (s, 3H), 1.88 (p, J=7.3 Hz, 2H), 1.74-1.64 (m, 2H), 1.61-1.45 (m, 4H).

¹³C NMR (126 MHz, CDCl₃) δ 163.14, 151.60, 150.19, 147.52, 142.95, 124.07, 123.67 (q, J=279.0 Hz), 122.67, 118.94, 116.76, 107.01, 99.00, 66.74 (q, J=34.9 Hz), 65.27, 62.30, 56.07, 53.38, 50.59, 45.39, 40.17, 30.45, 25.30, 24.76, 19.51, 16.77, 11.91.

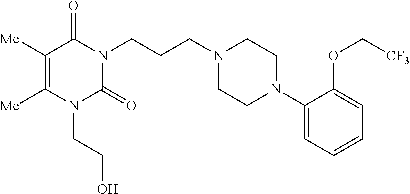 [SMILES: Cc1c(C)n(CCO)c(=O)n(CCCN2CCN(c3ccccc3OCC(F)(F)F)CC2)c1=O]

The above 2-tetrahydropyranyl ether (293.4 mg, 0.516 mmol) was dissolved in methanol (5.2 mL). Hydrogen chloride in isopropanol (155 μL, 0.774 mmol) was added in one portion via micropipette. The mixture was incubated for one hour at room temperature then triethylamine (145 μL, 1.03 mmol) was added (to quench excess HCl).

The reaction mixture was concentrated under reduced pressure. Saturated sodium bicarbonate (25 mL) was added, and the mixture was extracted with 3×25 mL portions of ethyl acetate. The combined organic layers were dried with MgSO₄, filtered, and concentrated under reduced pressure.

The product was recrystallized from 2:1 hexanes/isopropanol (4.5 mL), washed with isopropanol (0.5 mL) and hexanes (2 mL), and dried under reduced pressure to give the product as an off-white solid (167.7 mg, 0.346 mmol, 67% yield).

MP 133.4-134.5° C.

¹H NMR (500 MHz, CDCl₃) δ 7.01 (td, J=7.6, 1.5 Hz, 1H), 6.98-6.89 (m, 2H), 6.86 (dd, J=7.8, 1.6 Hz, 1H), 4.36 (q, J=8.3 Hz, 2H), 4.02 (t, J=5.2 Hz, 2H), 3.96 (t, J=7.4 Hz, 2H), 3.85 (t, J=5.2 Hz, 2H), 3.62 (s, 1H), 3.04 (br s, 4H), 2.58 (br s, 4H), 2.46 (t, J=7.2 Hz, 2H), 2.29 (s, 3H), 1.93 (s, 3H), 1.82 (p, J=7.3 Hz, 2H).

¹³C NMR (126 MHz, CDCl₃) δ 163.09, 152.18, 150.19, 147.54, 142.84, 124.07, 123.67 (q, J=277.4 Hz), 122.77, 118.93, 116.63, 107.26, 66.73 (q, J=34.9 Hz), 60.81, 56.03, 53.38, 50.53, 47.60, 40.20, 24.69, 16.89, 11.92.

### Synthesis of Compound 19

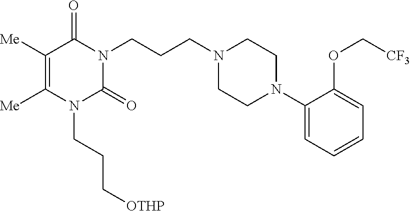

5,6-Dimethyl-3-[3-[4-[2-(2,2,2-trifluoroethoxy)phenyl]-1-piperazinyl]propyl]-2,4(1H,3H)-pyrimidinedione (429.7 mg, 0.976 mmol), cesium carbonate (635.7 mg, 1.95 mmol), and potassium iodide (161.9 mg, 0.976 mmol) were combined in a 25 mL RBF with magnetic stir bar. The flask was purged under argon then DMF (3.3 mL) was added. The suspension stirred for 10 minutes at 60° C. 2-(3-Bromopropoxy)tetrahydro-2H-pyran (265.2 mg, 1.19 mmol) was added in one portion, and the reaction continued to stir at 60° C. for 2 hours.

The reaction mixture was poured into 50 mL of half saturated brine. The mixture was extracted with 3×40 mL portions of 1:1 ethyl acetate/hexanes. The combined organic layers were dried with MgSO₄, filtered, and concentrated under reduced pressure.

The residue was purified with 5:95:1 to 50:50:1 ethyl acetate/hexanes/triethylamine and a 25 g silica gel cartridge. The product was collected as a clear oil (463.2 mg, 0.795 mmol, 81% yield)

¹H NMR (500 MHz, CDCl₃) δ 7.06-6.96 (m, 1H), 6.96-6.90 (m, 2H), 6.90-6.84 (m, 1H), 4.54 (t, J=3.9 Hz, 1H), 4.37 (q, J=8.4 Hz, 2H), 4.07-3.92 (m, 4H), 3.84-3.76 (m, 2H), 3.60-3.37 (m, 2H), 3.06 (br s, 4H), 2.59 (br s, 4H), 2.48 (t, J=7.2 Hz, 2H), 2.26 (s, 3H), 1.96 (s, 3H), 1.95-1.90 (m, 2H), 1.85 (p, J=7.4 Hz, 2H), 1.81-1.73 (m, 1H), 1.73-1.63 (m, 1H), 1.59-1.43 (m, 4H).

¹³C NMR (126 MHz, CDCl₃) δ 163.06, 151.62, 150.18, 146.57, 142.96, 124.06, 123.66 (q, J=278.4 Hz), 122.64, 118.91, 116.77, 107.24, 99.15, 66.73 (q, J=34.3 Hz), 64.68, 62.65, 56.08, 53.38, 50.61, 43.11, 40.20, 30.74, 29.21, 25.40, 24.77, 19.76, 16.01, 11.81.

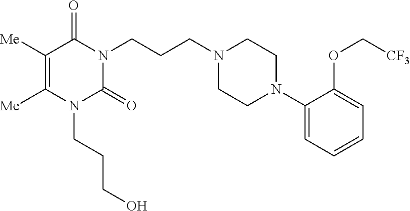 [SMILES: Cc1c(C)n(CCCO)c(=O)n(CCCN2CCN(c3ccccc3OCC(F)(F)F)CC2)c1=O]

The above 2-tetrahydropyranyl ether (377.6 mg, 0.648 mmol) was dissolved in methanol (6.5 mL). Hydrogen chloride in isopropanol (0.1944 mL, 0.972 mmol) was added in one portion via micropipette. The mixture was incubated for two hours at room temperature then triethylamine (182 μL, 1.30 mmol) was added (to quench excess HCl).

The reaction mixture was concentrated under reduced pressure. Saturated sodium bicarbonate (25 mL) was added, and the mixture was extracted with 3×25 mL portions of ethyl acetate. The combined organic layers were washed with 15 mL of brine, dried with MgSO₄, filtered, and concentrated under reduced pressure.

The product was recrystallized from 2:1 hexanes/isopropanol (4 mL), washed with hexanes (2 mL), and dried under reduced pressure to give the product as a white crystalline solid (266.2 mg, 0.534 mmol, 82% yield).

MP 115.0-115.7° C.

¹H NMR (500 MHz, CDCl₃) δ 7.06-7.01 (m, 1H), 6.97-6.93 (m, 2H), 6.91-6.88 (m, 1H), 4.39 (q, J=8.4 Hz, 2H), 4.11-4.01 (m, 4H), 3.60 (t, J=5.5 Hz, 2H), 3.39 (br s, 1H), 3.08 (br s, 4H), 2.61 (br s, 4H), 2.49 (t, J=7.3 Hz, 2H), 2.29 (s, 3H), 1.99 (s, 3H), 1.91-1.80 (m, 4H).

¹³C NMR (126 MHz, CDCl₃) δ 162.86, 152.68, 150.22, 146.39, 142.94, 124.10, 123.69 (q, J=278.5 Hz), 122.73, 118.96, 116.75, 108.10, 66.77 (q, J=35.2 Hz), 58.56, 56.04, 53.42, 50.62, 41.91, 40.42, 32.17, 24.78, 16.03, 11.87.

### Synthesis of Compound 20

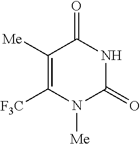 [SMILES: Cc1c(C(F)(F)F)n(C)c(=O)[nH]c1=O]

An oven dried 25 mL flask fitted with a magnetic stir bar and west condenser purged under argon was charged with 5-methyl-6-trifluoromethyluracil (350.8 mg, 1.81 mmol), acetonitrile (4.5 mL), and N,O-Bis(trimethylsilyl)acetamide (1.10 mL, 4.52 mmol). The suspension was stirred at room temperature for 3 hours. Dimethyl sulfate (686 μL, 7.23 mmol) was added to the reaction. The reaction was refluxed for 5 days.

The reaction mixture was poured into 50 mL of water and extracted with 3×25 mL portions of ethyl acetate. The combined organic layers were dried with MgSO₄, filtered, and concentrated under reduced pressure.

The residue was purified using a 12 g silica gel cartridge and 5 to 25% ethyl acetate in hexanes. The product was obtained as a white solid (62.2 mg, 0.299 mmol, 17% yield).

Rf=0.43 in 1:1 ethyl acetate/hexanes

¹H NMR (500 MHz, CDCl₃) δ 10.42 (s, 1H), 3.47 (q, J=2.2 Hz, 3H), 2.16 (q, J=4.7 Hz, 3H).

¹³C NMR (126 MHz, CDCl₃) δ 163.28, 150.88, 137.67 (q, J=32.7 Hz), 121.00 (q, J=277.8 Hz), 115.01 (q, J=2.1 Hz), 33.05 (q, J=5.0 Hz), 10.50 (q, J=4.9 Hz).

¹⁹F NMR (470 MHz, CDCl₃) δ −57.70. (unreferenced)

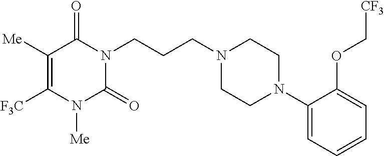 [SMILES: Cc1c(C(F)(F)F)n(C)c(=O)n(CCCN2CCN(c3ccccc3OCC(F)(F)F)CC2)c1=O]

1-methyl-6-trifluoromethyl-5-methyluracil, (50.6 mg, 0.243 mmol), cesium carbonate (158.4 mg, 0.486 mmol), and dimethylformamide (0.81 mL) were combined in an oven-dried 10 mL RBF with magnetic stir bar. The suspension stirred at 70° C. for 15 minutes. A 25 wt. % solution of mesylate in 1,4-dioxane (501 mg, 0.316 mmol) was added and the reaction was stirred at 70° C. for two hours.

The reaction mixture was poured into 20 mL of water and extracted with 3×30 mL portions of 1:1 ethyl acetate/hexanes. The combined organic layers were washed with 10 mL of brine, dried with MgSO₄, filtered, and concentrated under reduced pressure.

The crude product was purified with 10:90:1 to 40:60:1 ethyl acetate/hexanes/triethylamine and a 25 g silica gel cartridge. The product was obtained as a glassy residue (72.1 mg, 0.142 mmol, 58% yield).

¹H NMR (500 MHz, CDCl₃) δ 7.08-6.99 (m, 1H), 6.99-6.90 (m, 2H), 6.91-6.82 (m, 1H), 4.38 (q, J=8.3 Hz, 2H), 4.07 (t, J=7.3 Hz, 2H), 3.49 (q, J=2.3 Hz, 3H), 3.05 (br s, 4H), 2.59 (br s, 4H), 2.50 (t, J=7.0 Hz, 2H), 2.19 (q, J=4.5 Hz, 3H), 1.87 (p, J=7.1 Hz, 2H).

¹³C NMR (126 MHz, CDCl₃) δ 162.73, 151.24, 150.27, 142.95, 136.09 (q, J=32.6 Hz), 124.13, 123.73 (q, J=278.2 Hz), 122.81, 121.27 (q, J=277.5 Hz), 118.99, 116.70, 113.95 (q, J=2.2 Hz), 66.81 (q, J=35.0 Hz), 56.07, 53.45, 50.70, 41.42, 33.91 (q, J=5.0 Hz), 24.26, 11.30 (q, J=4.7 Hz).

¹⁹F NMR (470 MHz, CDCl₃) δ −57.44, −73.99. (unreferenced)

### Synthesis of Compound 21

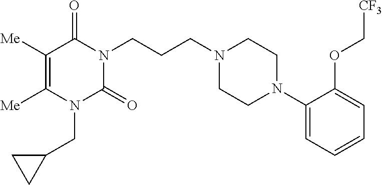 [SMILES: Cc1c(C)n(CC2CC2)c(=O)n(CCCN2CCN(c3ccccc3OCC(F)(F)F)CC2)c1=O]

5,6-Dimethyl-3-[3-[4-[2-(2,2,2-trifluoroethoxy)phenyl]-1-piperazinyl]propyl]-2,4(1H,3H)-pyrimidinedione (200.1 mg, 0.454 mmol), cesium carbonate (296 mg, 0.908 mmol), and potassium iodide (10 mg) were combined in a 10 mL RBF with magnetic stir bar. The flask was purged under argon for one minute then DMF (1.7 mL) was added. The suspension stirred for 10 minutes at room temperature. Cyclopropylmethyl bromide (74 mg, 0.545 mmol) was added in one portion and the reaction continued to stir at room temperature until the starting material was consumed.

The reaction mixture was poured into 30 mL of water. The mixture was extracted with 3×30 mL portions of 1:1 ethyl acetate/hexanes. The combined organic layers were dried with MgSO4, filtered, and concentrated under reduced pressure.

The residue was purified with 20:80:1 to 50:50:1 ethyl acetate/hexanes/triethylamine and a 12 g silica gel cartridge. The product was collected as a viscous pale yellow oil (186 mg, 0.376 mmol, 83% yield)

¹H NMR (500 MHz, CDCl₃) δ 7.03-6.98 (m, 1H), 6.95-6.90 (m, 2H), 6.90-6.84 (m, 1H), 4.37 (q, J=8.4 Hz, 2H), 4.02 (t, J=7.4 Hz, 2H), 3.82 (d, J=6.8 Hz, 2H), 3.05 (br s, 4H), 2.59 (br s, 4H), 2.48 (t, J=7.3 Hz, 2H), 2.28 (s, 3H), 1.97 (s, 3H), 1.86 (p, J=7.4 Hz, 2H), 1.08-0.95 (m, 1H), 0.61-0.50 (m, 2H), 0.47-0.39 (m, 2H).

¹³C NMR (126 MHz, CDCl₃) δ 163.12, 152.10, 150.22, 146.36, 142.99, 124.10, 123.70 (q, J=278.2 Hz), 122.70, 118.96, 116.77, 107.31, 66.76 (q, J=35.1 Hz), 56.15, 53.43, 50.64, 48.94, 40.34, 24.80, 16.43, 11.87, 10.97, 4.14.

¹⁹F NMR (470 MHz, CDCl₃) δ −73.96 (t, J=8.6 Hz). (unreferenced, 1H coupled)

### Synthesis of Compound 23

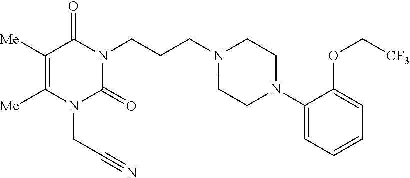 [SMILES: Cc1c(C)n(CC#N)c(=O)n(CCCN2CCN(c3ccccc3OCC(F)(F)F)CC2)c1=O]

5,6-Dimethyl-3-[3-[4-[2-(2,2,2-trifluoroethoxy)phenyl]-1-piperazinyl]propyl]-2,4(1H,3H)-pyrimidinedione (121.6 mg, 0.276 mmol) and cesium carbonate (179.9 mg, 0.552 mmol) were combined in a 10 mL RBF with magnetic stir bar. The flask was purged under argon for one minute then DMF (0.92 mL) was added. The suspension stirred for 15 minutes at room temperature. Bromoacetonitrile (25.0 μL, 0.359 mmol) was added in one portion and the reaction continued to stir at room temperature for 1 hour.

The reaction mixture was poured into 20 mL of water. The mixture was extracted with 3×20 mL portions of 1:1 ethyl acetate/hexanes. The combined organic layers were dried with MgSO₄, filtered, and concentrated under reduced pressure.

The residue was purified with 30:70:1 to 100:0:1 ethyl acetate/hexanes/triethylamine and a 12 g silica gel cartridge. The product was collected as a clear viscous oil (122.3 mg, 0.255 mmol, 92% yield).

Rf=0.24, 100:1 Ethyl acetate/triethylamine.

¹H NMR (500 MHz, CDCl₃) δ 7.07-6.97 (m, 1H), 6.98-6.89 (m, 2H), 6.90-6.81 (m, 1H), 4.83 (s, 2H), 4.36 (q, J=8.4 Hz, 2H), 4.01 (t, J=7.4 Hz, 2H), 3.05 (br s, 4H), 2.59 (br s, 4H), 2.48 (t, J=7.2 Hz, 2H), 2.34 (d, J=0.8 Hz, 3H), 1.98 (d, J=0.7 Hz, 3H), 1.85 (p, J=7.3 Hz, 2H).

¹³C NMR (126 MHz, CDCl₃) δ 162.41, 151.07, 150.14, 144.01, 142.80, 124.04, 123.66 (q, J=278.2 Hz), 122.74, 118.94, 116.56, 114.66, 109.41, 66.68 (q, J=35.0 Hz), 55.90, 53.34, 50.53, 40.75, 36.72, 32.65, 24.76, 24.48, 16.20, 11.87.

¹⁹F NMR (470 MHz, CDCl₃) δ −73.92 (t, J=8.6 Hz). (unreferenced, 1H coupled)

Administration of Compounds to α₁-ARS Receptor Sites Characterization of A_{1A}-ARS on Cardiac Myocytes

Decreased α_{1A}-AR expression has been demonstrated in myocardial cells from patients with dilated cardiomyopathy and congestive heart failure (CHF) and the α_{1A}-AR-selective agonist, A-61603 has been shown to reverse right ventricular failure in mice and block doxorubicin-induced cardiomyopathy and CHF in rats. The availability of an α1A-AR PET radiotracer may allow quantification of cardiac α_{1A}-ARs as a means of assessing disease severity, likelihood of responding to α_{1A}-AR agonist treatment, and/or assessing response to treatment over time.

### Involvement of α_{1A}-Ars in Substance Use Disorders

Blockade of brain ai-ARs with prazosin has been demonstrated to decrease the number of alcoholic drinks consumed per week and the number of heavy-drinking days per week in Veterans with alcohol use disorder, Veterans with alcohol use disorder plus PTSD, and active duty servicemembers with alcohol use disorder.

In addition, cocaine-depended individuals carrying the CC genotype of the rs1048101 variant of the α_{1A}-AR gene (ADRA1A) reported greater positive subjective effects (e.g., “desire”, “high”, “liking”, and “likely to use if given access”) than non-carriers in response to a 40 mg intravenous infusion of cocaine. As well, treatment with the ai-AR antagonist doxazosin has been shown to reduce cocaine use in dependent patients. Based on these findings, interrogation of brain ai-ARs with PET imaging in patients with alcohol use disorder or cocaine use disorder may help to elucidate the pathophysiology of these disorders.

α₁-ARS and Symptoms of Agitation and Aggression in Patients with Alzheimer'S Disease

In a double-blind, placebo-controlled study in elderly (80.6+/−11.2 years of age [mean+/−standard deviation]) patients with vs. without possible or probable Alzheimer's disease, treatment with the α₁-AR antagonist, prazosin (5.7+/−0.9 mg/day) resulted in significant reductions (vs. placebo) in symptoms of agitation and aggression, as assessed by the Brief Psychiatric Rating Scale, the Neuropsychiatric Inventory, and the Clinical Global Impression of Change scales. PET imaging of brain ai-ARs in patients with Alzheimer's disease may help to elucidate the pathophysiology of disease-related agitation and aggression, symptoms that are more likely to result in nursing home placement than cognitive impairment per se.

### α₁-ARS and Post-Traumatic Headaches

In a study of 48 veterans and active-duty servicemembers with mild traumatic brain injury related chronic headaches, treatment with prazosin in doses up to 25 mg/day resulted in a significant reduction in headache frequency and headache impact scores over a follow-up period of twelve-weeks with only a 6% drop-out rate. PET imaging of brain ai-ARs may help to elucidate the mechanism by which prazosin treatment mitigates headache pain.

α₁-ARS and Urinary Outflow Obstruction in Patients with Benign Prostatic Hypertrophy

Prazosin and other, more α1A-AR-selective antagonists have been the mainstay for treatment of urinary outflow obstruction in men with benign prostatic hypertrophy (BPH). As well, a more recent study demonstrated that prazosin sensitized PC-3 and LNCaP prostate cancer cells to docetaxel in vitro. This suggests that PET imaging of α₁-ARs may help with the diagnosis of BPH, tracking responses to treatment with α₁-AR antagonists, and determine whether prazosin, and possibly other α₁-AR antagonists, sensitize prostate cancer cells to the effects of docetaxel, and possibly other chemotherapeutic agents, in vivo.

### Other Approaches to the Problem

There have been multiple attempts to develop ai-AR PET radiotracer compounds for use in both CNS and cardiac imaging. Drugs investigated include: [¹¹C]prazosin, [¹²⁵I]HEAT (BE-2254), [¹¹C]GB67, [¹¹C]RN5, [¹²⁵I]L-762459, Tc-99m-labeled derivatives of WAY-100635, and ¹¹C-labeled derivatives of the atypical antipsychotic drugs sertindole, octoclothepin, and iloperidone. However, none of these compounds have proven suitable for development as a radiotracer due to drawbacks such as: (1) insufficient uptake into the brain; (2) high levels of non-specific binding; (3) high levels of binding to 5-HT (serotonin) 1A, 2A, 2B, and/or 2C receptors, all of which have molecular structures closely related to that of the ai-AR. Similar “off-target” binding to 5-HT receptors has prevented development of PET radiotracers based on other ai-AR antagonists.

### Methods of Use

The radiolabeled compounds described in this disclosure may be used in various methods for in vivo imaging of tissues, developing radiolabeled compounds for treatment or assessment of medical conditions, predicting responsiveness to an α1-AR-targeted drug for assessment of a treatment protocol for a medical condition, and/or assessing a treatment protocol for a subject with a medical condition.

As used herein, “subject” refers to an animal having a medical condition or who is being assessed for a treatment protocol for a medical condition. The animal may be a human animal or a non-human animal. The subject may also refer to a human patient.

In an aspect, the present disclosure provides a method for assessment of a treatment protocol for a subject with a medical condition that is treatable by targeting an al-AR receptor, the method comprising: screening the subject for prediction of responsiveness of the subject to an α1-AR-targeted drug, wherein the screening comprises: administering an effective amount of a radiolabeled compound of Formula (I), or a pharmaceutical salt thereof, to the subject, wherein Formula (I) is

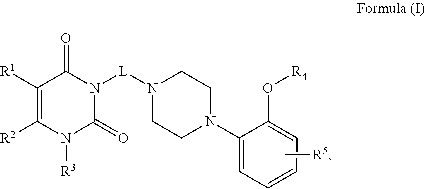

wherein: R¹ is a C₁-C₆ alkyl or a C₁-C₆ haloalkyl; R² is H, a C₁-C₆ alkyl, or a C₁-C₆ haloalkyl; or R¹ and R² together form a 5- or 6-membered cycloalkyl ring, a substituted or unsubstituted thiophenyl, or a substituted or unsubstituted phenyl; R³ is H, a C₁-C₆ alkyl, a C₁-C₆ haloalkyl, a C₁-C₆ hydroxyl alkyl, a C₃-C₆ cycloalkyl, a C₁-C₆ alkylnitrile, or an C₁-C₆ alkylcycloalkyl; R⁴ is C₁-C₆ alkyl or C₁-C₆ haloalkyl; R⁵ is H or halide; and L is a 2 to 10 carbon linking group; provided that: when R¹ is Me, R² is Me, R³ is H, R⁴ is —OCH₂CF₃, and L is —CH₂—CH₂—CH₂—, R⁵ is not H; and when R¹ is Me, R² is H, R³ is H, R⁴ is —OCH₂CF₃, and L is —CH₂—CH₂—CH₂—, R⁵ is not H; and radio-imaging at least a portion of a site of an α1-AR receptor in a subject, wherein a resultant radiograph corresponds to a predicted responsiveness of the subject to the radiolabeled compound, or a pharmaceutically acceptable salt thereof, and administering an effective amount of the α1-AR-targeted drug to the subject based on the predicted responsiveness, wherein the α1-AR-targeted drug is a non-radiolabeled derivative of the radiolabeled compound of Formula (I).

In some embodiments, R¹ is methyl or isopropyl.

In some embodiments, R² is H or Me.

In some embodiments, R² is CH₂F, CHF₂, or CF₃.

In some embodiments, R¹ and R² together form

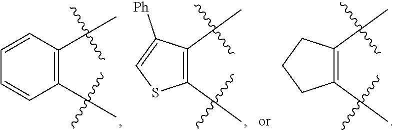 [SMILES: *c1ccccc1* ; *c1scc(-c2ccccc2)c1* ; *C1=C(*)CCC1]

In some embodiments, R³ is H, CH₂CN, methyl, ethyl, propyl, or isopropyl.

In some embodiments, R³ is fluoromethyl, fluoroethyl, or fluoropropyl.

In some embodiments, R³ is hydroxyethyl or hydroxypropyl.

In some embodiments, R³ is cyclopropyl or methyl cyclopropyl.

In some embodiments, R⁴ is Me or CH₂CF₃.

In some embodiments, R⁵ is F.

In some embodiments, L is —(CH₂)ₙ, wherein n=2 or 3. In some embodiments, L is a C₂-C₃ alkylene group which is optionally substituted by 1, 2, 3, 4, 5, or 6 carbon-containing substituents. For instance, L may be substituted with a methyl group, an ethyl group, a propyl group, an isopropyl group, a cycloalkyl group, or a phenyl group. L may be further substituted by a halo group. L may be a linear alkylene group or a branched alkylene group.

In some embodiments, the radiolabeled compound of Formula (I) is selected from the group consisting of:

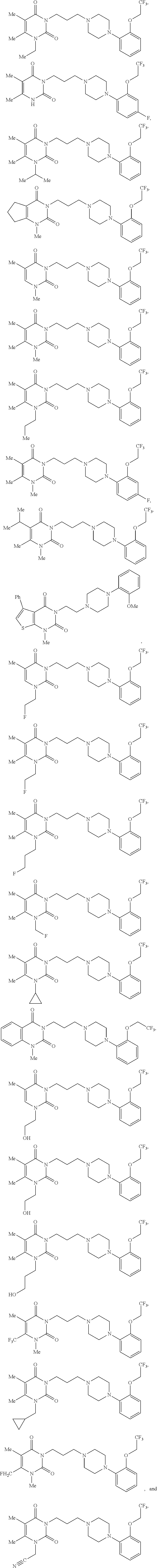

or pharmaceutically acceptable salts thereof.

In some embodiments, the radiolabeled compound comprises a carbon-11 (¹¹C), a carbon-14 (¹⁴C), a tritium (³H), or fluorine-18 (¹⁸F) radiolabel.

In some embodiments, a carbon of the alkyl, haloalkyl, hydroxyl alkyl, cycloalkyl, or alkyl-cycloalkyl group of R³ is ¹¹C.

In some embodiments, a halide of the haloalkyl group of R⁴ is ¹⁸F.

In some embodiments, a halide of the haloalkyl group of R² or a halide of the linear or branched haloalkyl group of R³ is ¹⁸F.

In some embodiments, the radio-imaging comprises positron emission tomography (PET) scanning.

In an aspect, the present disclosure provides a method for in vivo imaging of a tissue expressing an α1-AR receptor in a subject, the method comprising: administering an effective amount of a radiolabeled compound of Formula (I), or a pharmaceutical salt thereof, to the subject, wherein Formula (I) is

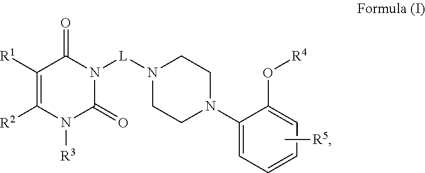

wherein: R¹ is a C₁-C₆ alkyl or a C₁-C₆ haloalkyl; R² is H, a C₁-C₆ alkyl, or a C₁-C₆ haloalkyl; or R¹ and R² together form a 5- or 6-membered cycloalkyl ring, a substituted or unsubstituted thiophenyl, or a substituted or unsubstituted phenyl; R³ is H, a C₁-C₆ alkyl, a C₁-C₆ haloalkyl, a C₁-C₆ hydroxyl alkyl, a C₃-C₆ cycloalkyl, a C₁-C₆ alkylnitrile, or an C₁-C₆ alkylcycloalkyl; R⁴ is C₁-C₆ alkyl or C₁-C₆ haloalkyl; R⁵ is H or halide; and L is a 2 to 10 carbon linking group; provided that: when R¹ is Me, R² is Me, R³ is H, R⁴ is —OCH₂CF₃, and L is —CH₂—CH₂—CH₂—, R⁵ is not H; and when R¹ is Me, R² is H, R³ is H, R⁴ is —OCH₂CF₃, and L is —CH₂—CH₂—CH₂—, R⁵ is not H; and radio-imaging at least a portion of the subject, wherein a position of a radio signal of a resultant radiograph corresponds to a site of the α1-AR receptor in the subject.

As used herein, “predicted responsiveness” refers to the degree to which the symptoms related to a medical condition of a patient administered an α1-AR-targeted drug will be treated, reduced, improved, alleviated, or otherwise affected by treatment with the al-AR-targeted drug.

Without wishing to be bound by any particular theory, methods according to this aspect may advantageously allow for a diagnostic determination of the degree to which a particular patient responds to a particular α1-AR-targeted drug. As this can vary between patients, and as responsiveness is related to effectiveness of treatment, these methods may improve patient outcomes.

In an aspect, the present disclosure provides a method for predicting responsiveness of an α1-AR receptor a subject to an α1-AR-targeted drug, for assessment of a treatment protocol for a medical condition, the method comprising: administering an effective amount of any of the radiolabeled compounds of the present disclosure, or a pharmaceutically acceptable salt thereof, to the subject; and radio-imaging at least a portion of a site of an α1-AR receptor in the subject, wherein a resultant radiograph comprises a radioactivity detection, a nuclear imaging scan, or another imaging, and corresponds to a predicted positive responsiveness of the subject to the radiolabeled compound, or a pharmaceutically acceptable salt thereof.

In some embodiments, the medical condition is selected from the group consisting of: post-traumatic stress disorder (PTSD), post-traumatic headaches, behavioral dysfunction (agitation and/or aggression) in Alzheimer's disease, alcohol use disorder, urinary dysfunction, autonomic dysreflexia in patients with spinal cord injuries, congestive heart failure (CHF), and any combination thereof.

In some embodiments, the medical condition is selected from the group consisting of: any disease characterized by autonomic instability or autonomic dysfunction or dysautonomia or adrenergic autonomic failure or Parkinsons's disease, spinal cord injury, diabetes mellitus, chronic fatigue syndrome, cancer, neuroblastoma, glioblastoma, a substance use disorder, cocaine use disorder, cannabis use disorder, a neuropsychiatric disorder, cardiac dysfunction, a disorder of prostate and/or urinary incontinence, a metabolic disorder, an evaluation of brown adipose tissue, and any combination thereof.

In some embodiments, the medical condition is selected from the group consisting of: post-traumatic stress disorder (PTSD), post-traumatic headaches, behavioral dysfunction (agitation and/or aggression) in Alzheimer's disease, alcohol use disorder, cannabis use disorder, urinary dysfunction, autonomic dysreflexia in patients with spinal cord injuries, congestive heart failure (CHF), any disease characterized by adrenergic autonomic failure, overactivity, or dysfunction, evaluation of brown adipose tissue, and any combination thereof.

In some embodiments, the tissue is selected from the group consisting of: central nervous system tissue, cardiovascular system tissue, heart tissue, bladder tissue, prostate tissue, muscle tissue, adipose tissue, brown adipose tissue, and any combination thereof.

In some embodiments, the α1-AR-targeted drug is a α1A-AR-targeted drug, an α1B-AR-targeted drug, an α1D-AR-targeted drug, or any combination thereof.

In some embodiments, the α1-AR receptor is a α1A-AR receptor, an α1B-AR receptor, or an α1D-AR receptor.

### EXAMPLES

### Example 1: Administration of [C-11]ARMI to Rats

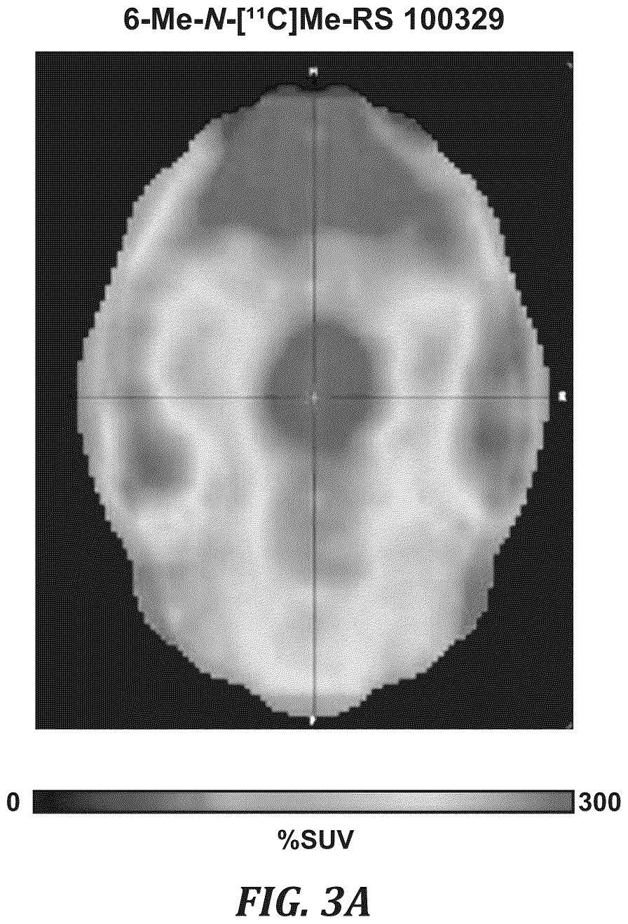

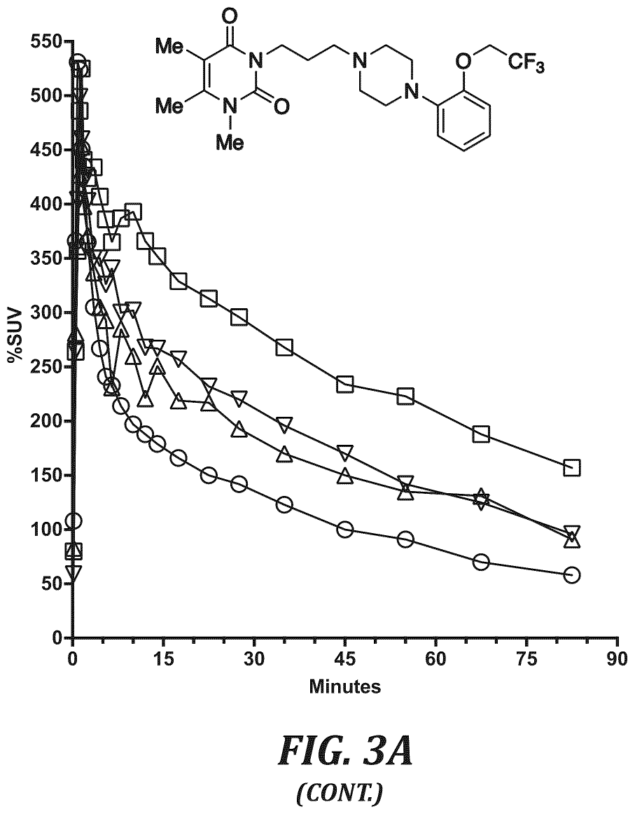

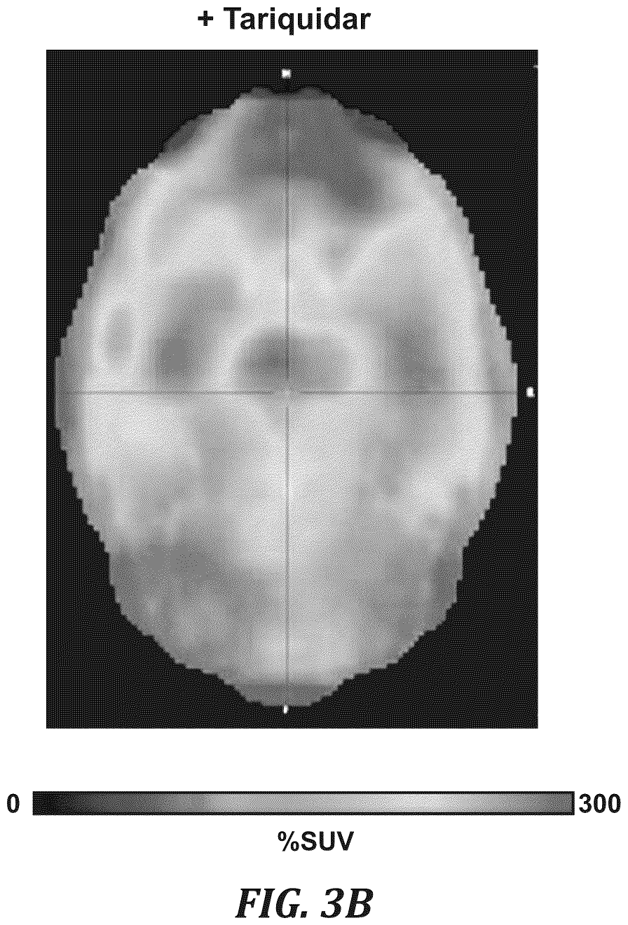

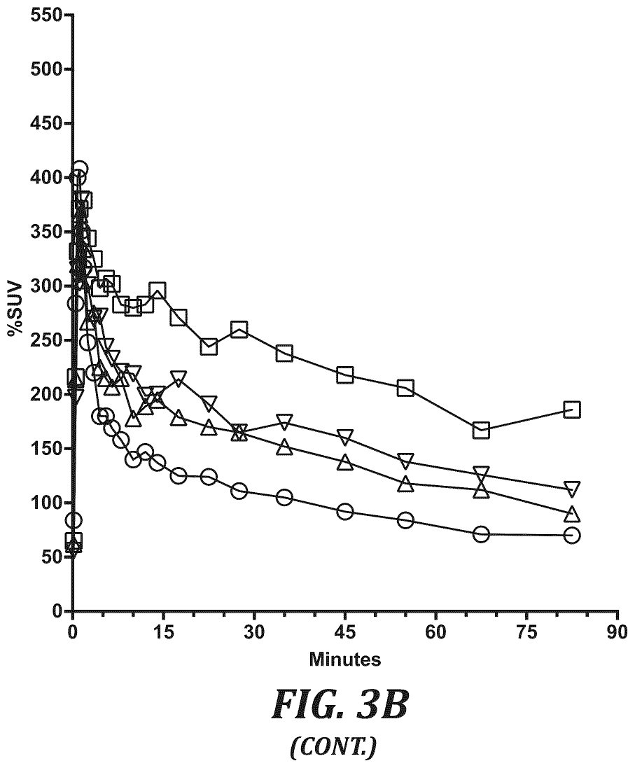

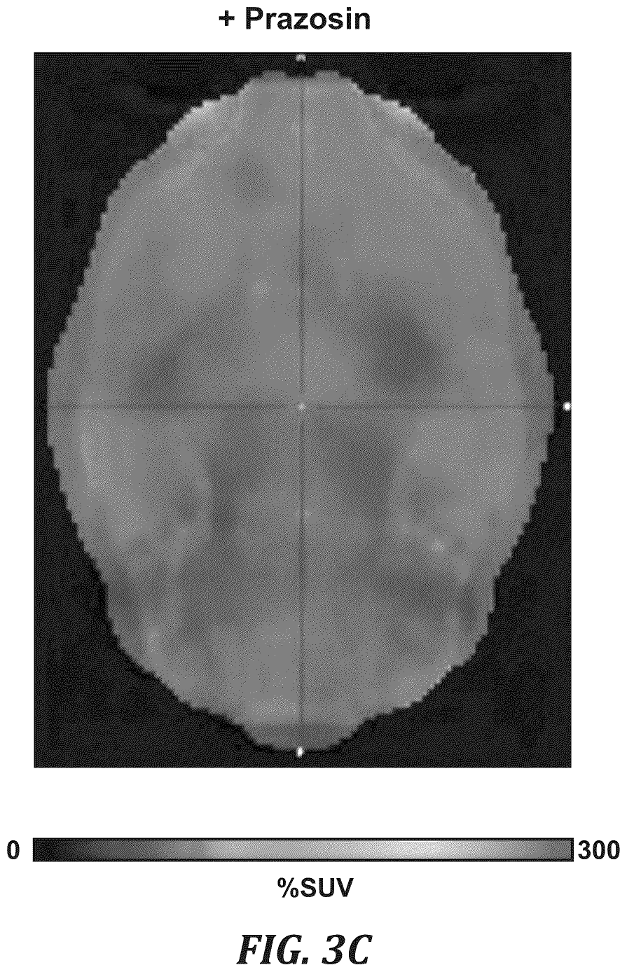

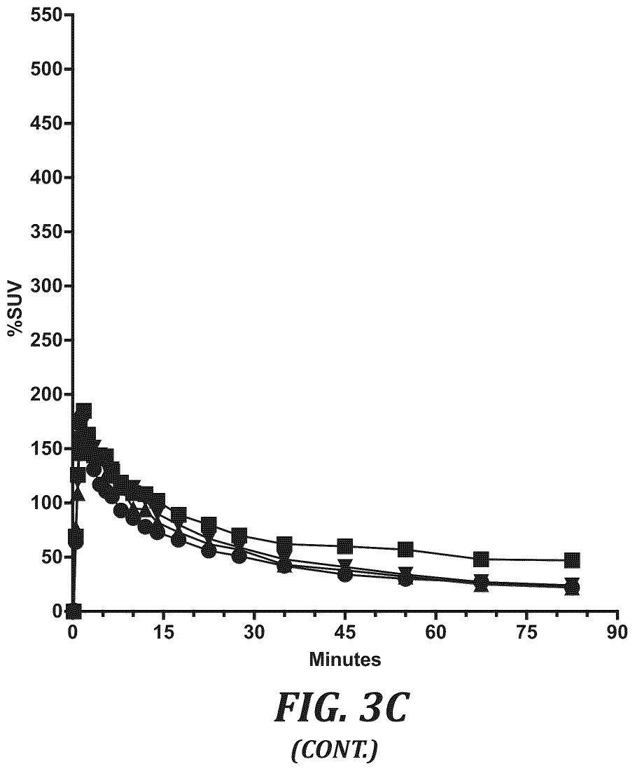

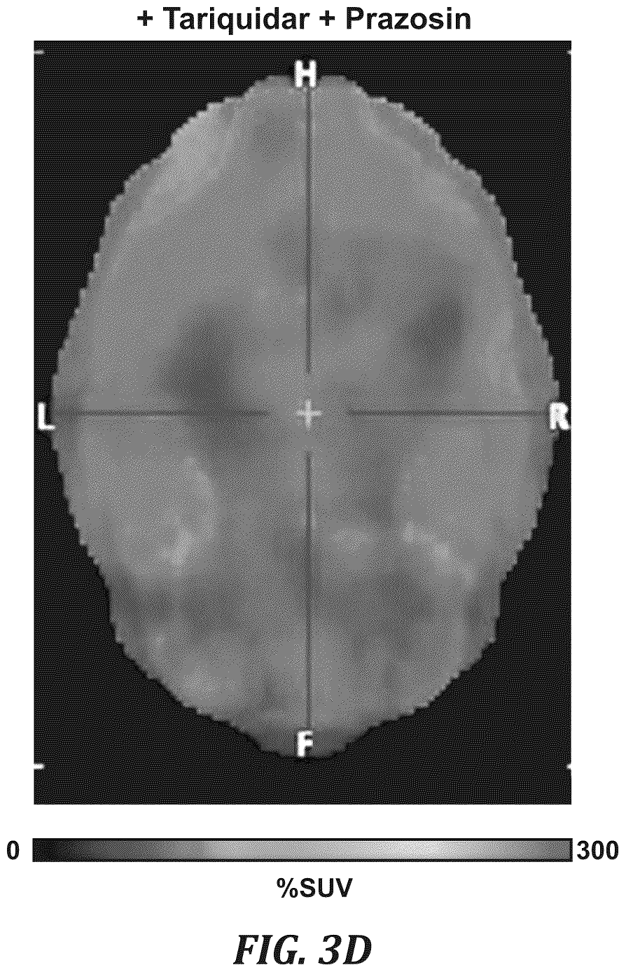

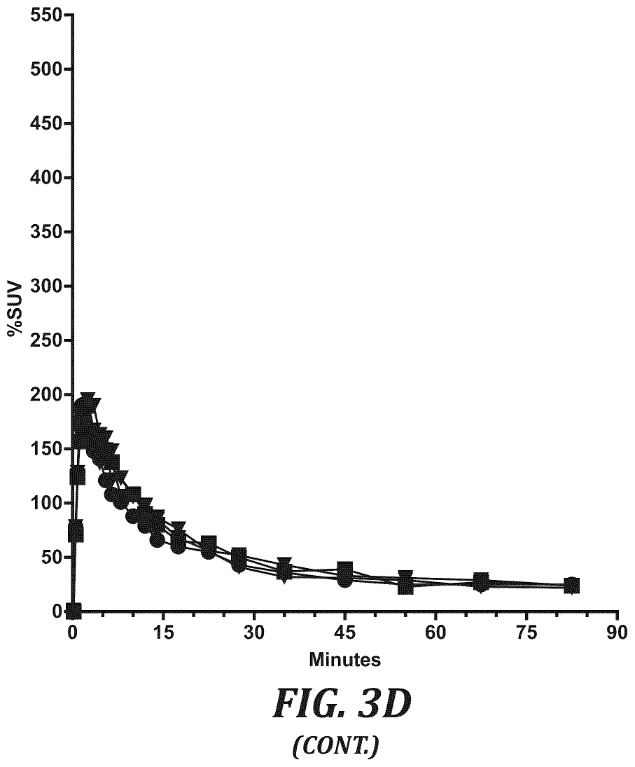

Pharmaceutically effective dosages of tariquidar, prazosin, and a mixture of Tariquidar and Prazosin were administered to rats, followed in injection of [C-11]ARMI to measure the radiolabeling of rat brain α_{1A}-ARs. FIG. 3 depicts PET images and inset time-activity curves of [C-11]ARMI in rat brains, demonstrating selectivity for ai-AR (by prazosin blockade) and lack of sensitivity for P-gp efflux transport (by tariquidar blockade). All images are summed from 0-90 minutes after radiotracer injection, normalized for body weight and injected radioactivity (standardized uptake value×100% [SUV %]), masked for extracerebral radioactivity, and are in rostral-caudal axial orientation, with frontal cortex superior to cerebellum. Insets have Time Activity Curves (TACs) of selected rat brain regions following injection of [C-11]ARMI over the course of a 90-min scan, expressed as SUV %. Regions with previously described high (frontal cortex [squares]), moderate (striatum [inverted triangles], hippocampus [triangles]), and low (cerebellum [circles]) 1A-AR expression are shown. TACs provide quantitative measures of radioactivity over time, with SUV of 100% representing even distribution of radioactivity throughout the body (i.e., no specific accumulation by receptor binding).

In this regard, FIGS. 3A-3D demonstrate the effectiveness of the radiolabeled derivatives of any of the compounds described herein for imaging α_{1A}-AR binding and analysis of the predicted responsiveness of a patient to an α_{1A}-AR-targeting drug, as in any of the methods described herein.

FIG. 3A depicts [C-11]ARMI in rat brain, demonstrating a radioactivity distribution consistent with that of reported and separately measured in vitro distribution of α_{1A}-AR. TACs reflect varying levels of radioactivity accumulation ordinal to known α_{1A}-AR distribution. Decreasing values over time suggests the radiotracer demonstrates reversible binding at the target.

FIG. 3B depicts [C-11]ARMI one hour after intraperitoneal (ip) injection of the P-glycoprotein (P-gp) efflux transporter inhibitor, tariquidar (20 mg/kg). Absence of change between baseline (FIG. 1A) and tariquidar blockade of P-gp (FIG. 1B) shows that [C-11]ARMI is not a substrate for the P-gp efflux transporter.

FIG. 3C depicts [C-11]ARMI one hour after ip injection of the subtype nonselective α₁-AR antagonist, prazosin (30 mg/kg). Prazosin blocked radioactivity distribution and accumulation suggesting [C-11]ARMI is selectively binding to one or more subtypes of the α₁-AR. This is also supported by the fact that prazosin reduced binding in all brain regions, reducing radioactivity to the same low level, demonstrating [C-11]ARMI has low non-specific binding (i.e., good specificity). Comparison of TACs in FIG. 3A and FIG. 3C implies the radiotracer has ~2/3 specific binding.

FIG. 3D depicts [C-11]ARMI one hour after ip injection of both tariquidar (20 mg/kg) and prazosin (30 mg/kg), further confirming the main finding of FIG. 3C by eliminating any potential confound by P-gp efflux transport of the radioligand or prazosin.

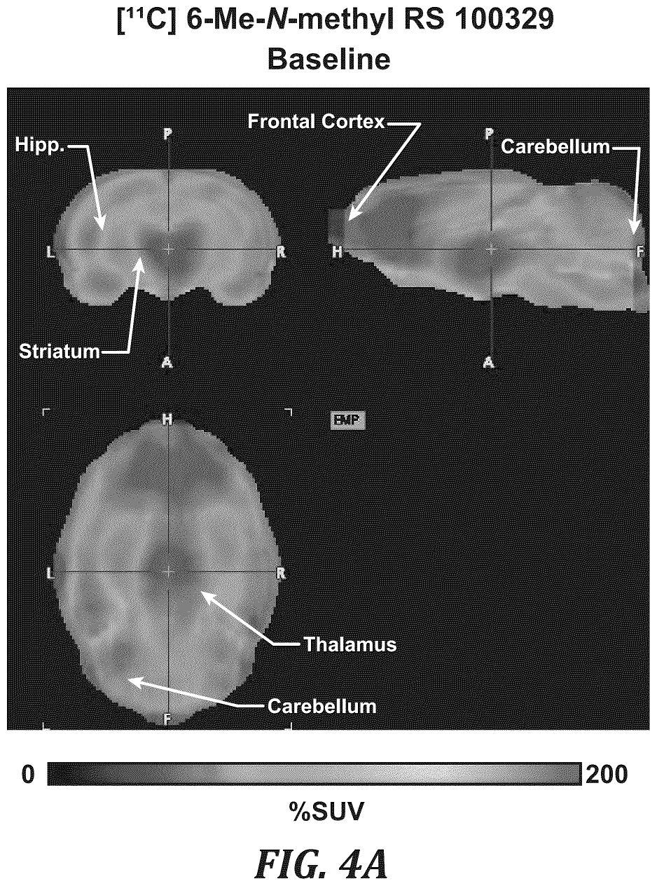

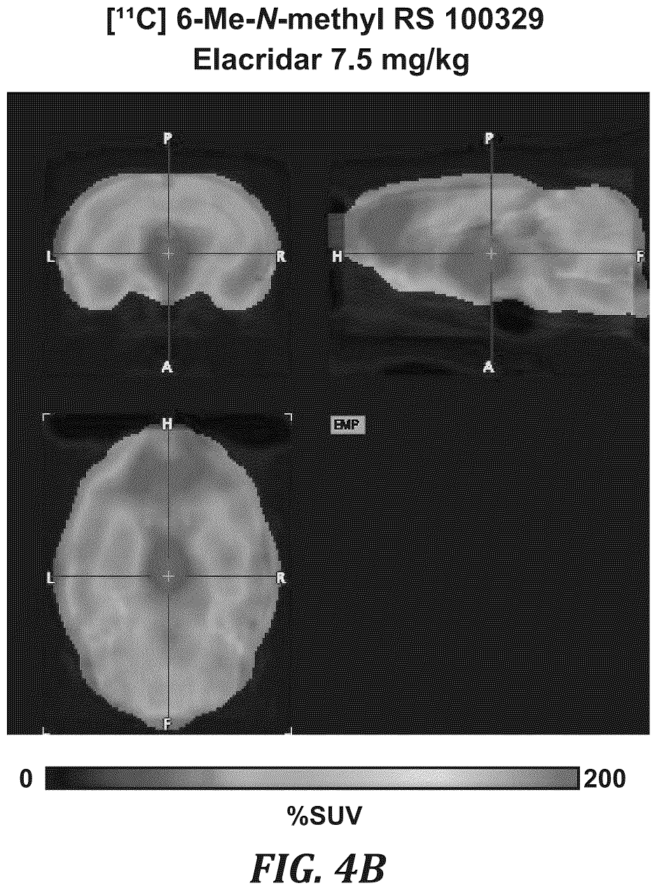

FIG. 4A and FIG. 4B demonstrate that [C-11]ARMI uptake in a rat brain is not affected by inhibition of drug-efflux transporters at the blood-brain barrier (BBB).

FIG. 4A depicts coronal, sagittal, and axial views (left to right) of [C-11]ARMI uptake and distribution under baseline conditions.

FIG. 4B depicts coronal, sagittal, and axial views of [C-11]ARMI uptake after inhibition of the dual P-glycoprotein (P-gp) and breast cancer resistance protein (BCRP) efflux transporters by ip injection of elacridar (7.5 mg/kg) one hour prior to radiotracer administration. Comparison of TACs (not shown) from baseline and elacridar-block scans is ~4%, reflecting an unsubstantial difference between conditions.

### Example 2: Binding of [C-11]ARMI in Human Embryonic Kidney (HEK) Cells

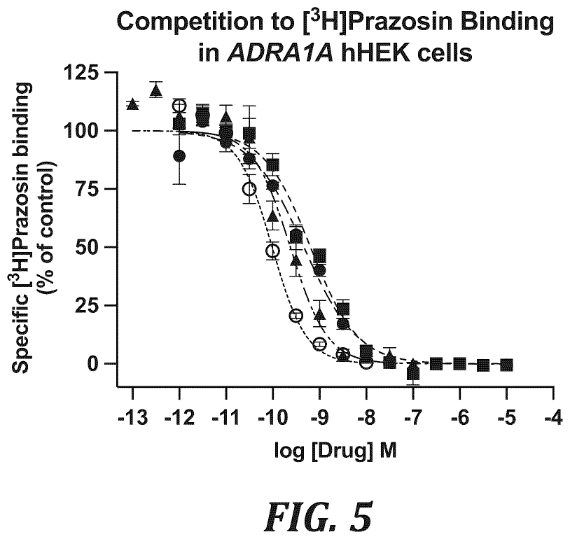

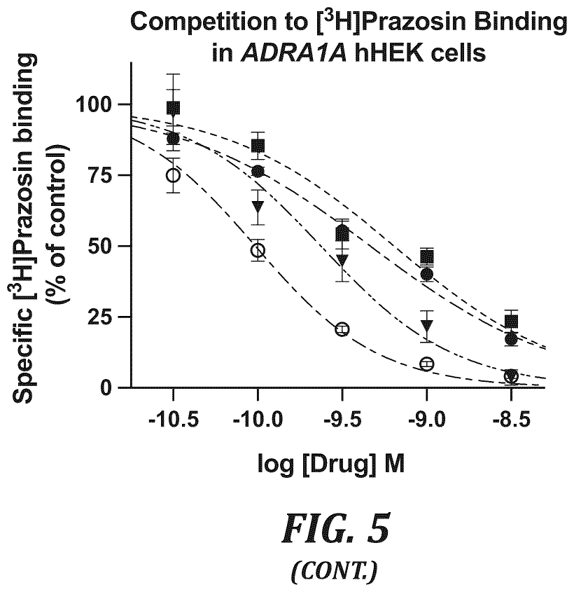

FIG. 5 depicts the inhibition of [³H]prazosin binding to human α_{1A}-ARs expressed in HEK cells by increasing concentrations of ARMI (triangles), compared to reference α₁-AR antagonists prazosin (closed circles) and tamsulosin (open circles). Also shown is inhibition of binding of our previously disclosed 1A-AR antagonist, N-Me-RS100329 (squares; Compound 5). Inset shows expanded data for clarity. See Table 2 for associated Ki values, and Table 3 for selectivity screening at potential Off-target receptors. Values are means based on four (ARMI, prazosin) or three (tamsulosin) independent experiments with duplicate determinations.

TABLE 2
Ki values for ARMI, N-Me-RS100329, and reference α₁-AR
antagonists

| Ki value (nM) | Compound |
|---|---|
| 0.0494 ± 0.0095 | ARMI-HCl |
| 0.129 ± 0.017 | N-Me-RS100329 (prior art) |
| 0.110 ± 0.023 | Prazosin (reference standard) |
| 0.0237 ± 0.0031 | Tamsulosin (reference standard |

TABLE 3
Selectivity screening at potential “Off-Target” receptors at
which ARMI inhibited binding of receptor-specific
antagonist by 50 percent or more.

| | ARMI-HCL Concentration | |
|---|---|---|
| Receptor | 10 nM | 1,000 nM |
| α_{1B}-AR | No | Yes |
| α_{1D}-AR | No | Yes |
| α_{2A}-AR | No | Yes |
| α_{2B}-AR | No | Yes |
| α_{2C}-AR | No | Yes |
| Serotonin 5HT-1A | No | Yes |
| Serotonin 5HT-2A | No | No |
| Serotonin 5HT-2B | No | Yes |
| Serotonin 5HT-2C | No | No |
| Dopamine D-1 | No | No |
| Dopamine D-2 | No | No |
| Dopamine D-3 | No | Yes |
| Dopamine D-4.4 | No | Yes |
| Norepinephrine transporter | No | No |

Less than 50% inhibition of ligand binding by ARMI-HCl (Ki<0.05 nM) at 10 nM corresponds to greater than 200-fold greater affinity for α_{1A}-AR vs. listed “off-target” receptors.

### Example 3: Binding of [F-18] Compound 12 to Rats

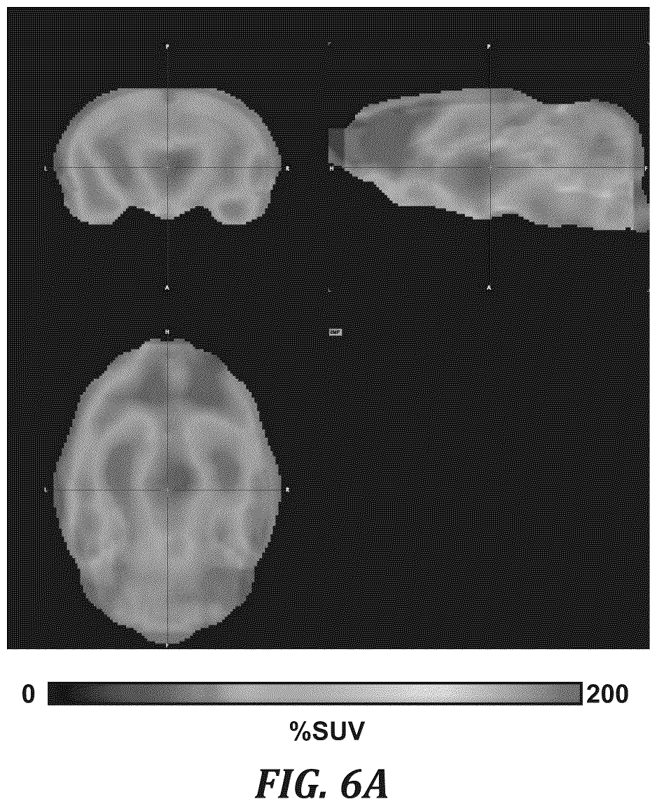

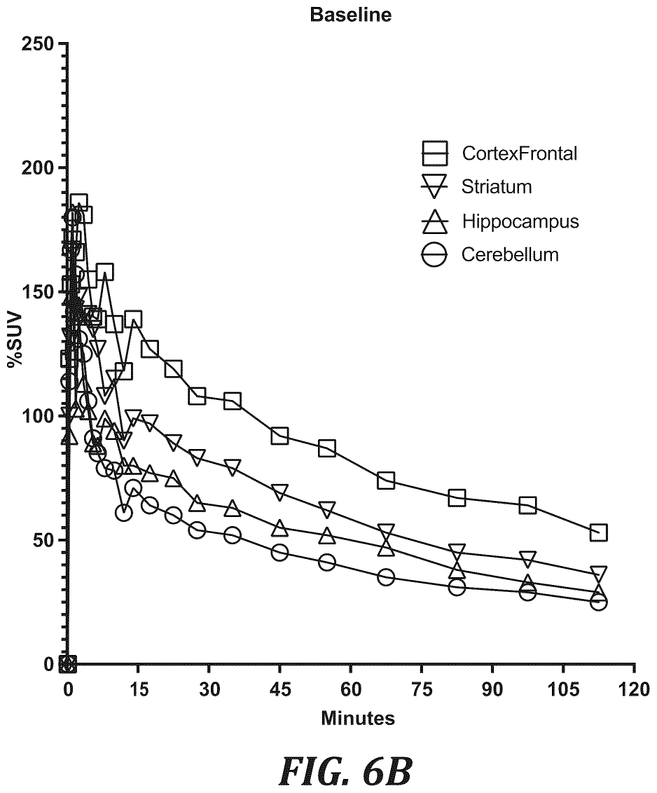

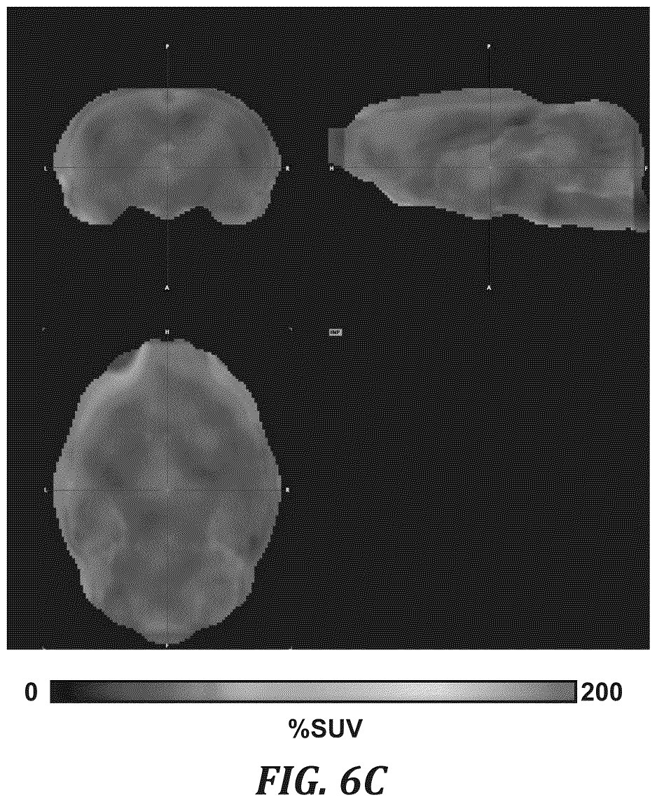

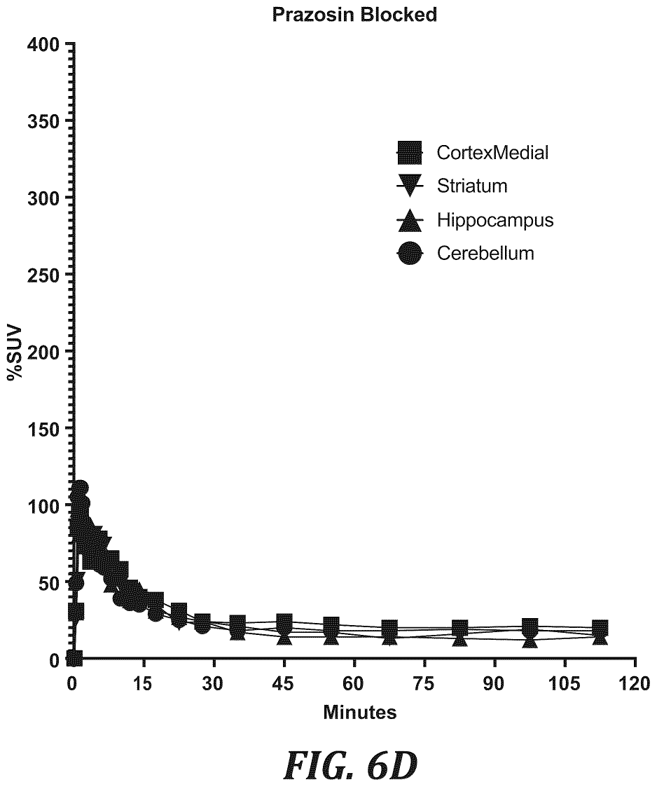

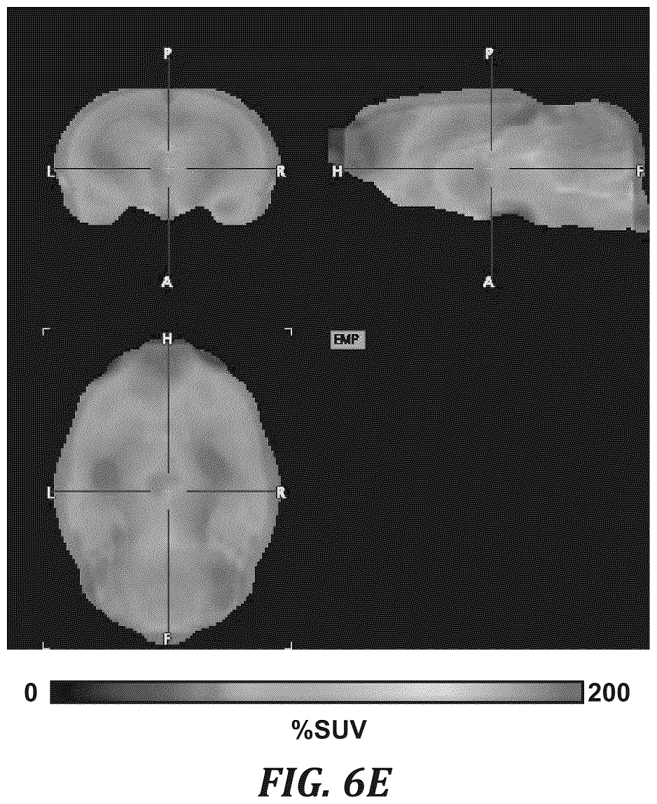

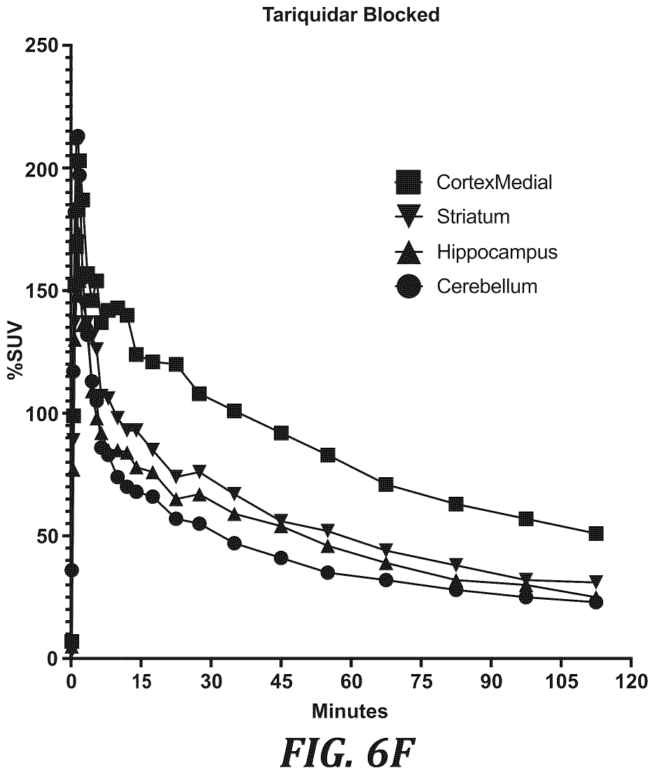

FIG. 6A depicts coronal (upper left), sagittal (upper right), and axial (lower left) views of Compound 12 (with a [F-18]fluoroethyl substituent on the amine) uptake and distribution under baseline conditions.

FIG. 6B depicts TACs of Compound 12 (with a [F-18]fluoroethyl substituent on the amine) uptake and distribution under baseline conditions, which reflect varying levels of radioactivity accumulation ordinal to known α_{1A}-AR distribution.

FIG. 6C depicts coronal (upper left), sagittal (upper right), and axial (lower left) views of Compound 12 (with a [F-18]fluoroethyl substituent on the amine) uptake and distribution under prazosin blocking conditions.

FIG. 6D depicts TACs of Compound 12 (with a [F-18]fluoroethyl substituent on the amine) uptake and distribution under prazosin blocking conditions. This demonstrates analogous concepts to that depicted in FIG. 3C. Here, the difference is 55%.

FIG. 6E depicts coronal (upper left), sagittal (upper right), and axial (lower left) views of Compound 12 (with a [F-18]fluoroethyl substituent on the amine) uptake and distribution under tariquidar blocking conditions.

FIG. 6F depicts TACs of Compound 12 (with a [F-18]fluoroethyl substituent on the amine) uptake and distribution under tariquidar blocking conditions. This demonstrates analogous concepts to FIG. 3B, and that there is no significant difference between the baseline and tariquidar.

### Non-Limiting Embodiments

While general features of the disclosure are described and shown and particular features of the disclosure are set forth in the claims, the following non-limiting embodiments relate to features, and combinations of features, that are explicitly envisioned as being part of the disclosure. The following non-limiting embodiments contain elements that are modular and can be combined with each other in any number, order, or combination to form a new non-limiting embodiment, which can itself be further combined with other non-limiting embodiments.

Embodiment 1. A method for assessment of a treatment protocol for a subject with a medical condition that is treatable by targeting an α1-AR receptor, the method comprising: screening the subject for prediction of responsiveness of the subject to an al-AR-targeted drug, wherein the screening comprises: administering an effective amount of a radiolabeled compound of Formula (I), or a pharmaceutical salt thereof, to the subject, wherein Formula (I) is

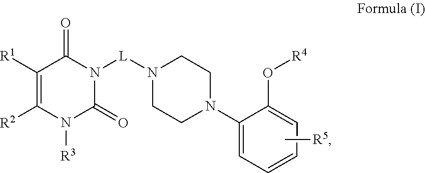

wherein: R¹ is a C₁-C₆ alkyl or a C₁-C₆ haloalkyl; R² is H, a C₁-C₆ alkyl, or a C₁-C₆ haloalkyl; or R¹ and R² together form a 5- or 6-membered cycloalkyl ring, a substituted or unsubstituted thiophenyl, or a substituted or unsubstituted phenyl; R³ is H, a C₁-C₆ alkyl, a C₁-C₆ haloalkyl, a C₁-C₆ hydroxyl alkyl, a C₃-C₆ cycloalkyl, a C₁-C₆ alkylnitrile, or an C₁-C₆ alkylcycloalkyl; R⁴ is C₁-C₆ alkyl or C₁-C₆ haloalkyl; R⁵ is H or halide; and L is a 2 to 10 carbon linking group; provided that: when R¹ is Me, R² is Me, R³ is H, R⁴ is —OCH₂CF₃, and L is —CH₂—CH₂—CH₂—, R⁵ is not H; and when R¹ is Me, R² is H, R³ is H, R⁴ is —OCH₂CF₃, and L is —CH₂—CH₂—CH₂—, R⁵ is not H; and radio-imaging at least a portion of a site of an α1-AR receptor in a subject, wherein a resultant radiograph corresponds to a predicted responsiveness of the subject to the radiolabeled compound, or a pharmaceutically acceptable salt thereof, and administering an effective amount of the α1-AR-targeted drug to the subject based on the predicted responsiveness, wherein the α1-AR-targeted drug is a non-radiolabeled derivative of the radiolabeled compound of Formula (I).

Embodiment 2. The method of Embodiment 1 or any other Embodiment, wherein R¹ is methyl or isopropyl.

Embodiment 3. The method of Embodiment 1 or Embodiment 2 or any other Embodiment, wherein R² is H or Me.

Embodiment 4. The method of Embodiments 1-3 or any other Embodiment, wherein R² is CH₂F, CHF₂, or CF₃.

Embodiment 5. The method of Embodiments 1-4 or any other Embodiment, wherein R¹ and R² together form

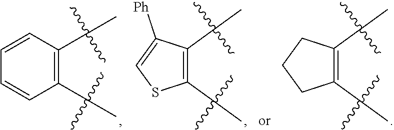 [SMILES: *c1ccccc1* ; *c1scc(-c2ccccc2)c1* ; *C1=C(*)CCC1]

Embodiment 6. The method of Embodiments 1-5 or any other Embodiment, wherein R³ is H, CH₂CN, methyl, ethyl, propyl, or isopropyl.

Embodiment 7. The method of Embodiments 1-6 or any other Embodiment, wherein R³ is fluoromethyl, fluoroethyl, or fluoropropyl.

Embodiment 8. The method of Embodiments 1-7 or any other Embodiment, wherein R³ is hydroxyethyl or hydroxypropyl.

Embodiment 9. The method of Embodiments 1-8 or any other Embodiment, wherein R³ is cyclopropyl or methyl cyclopropyl.

Embodiment 10. The method of Embodiments 1-9 or any other Embodiment, wherein R⁴ is Me or CH₂CF₃.

Embodiment 11. The method of Embodiments 1-10 or any other Embodiment, wherein R⁵ is F.

Embodiment 12. The method of Embodiments 1-13 or any other Embodiment, wherein L is —(CH₂)ₙ, wherein n=2 or 3.

Embodiment 13. The method of Embodiments 1-12 or any other Embodiment, wherein the radiolabeled compound of Formula (I) is selected from the group consisting of:

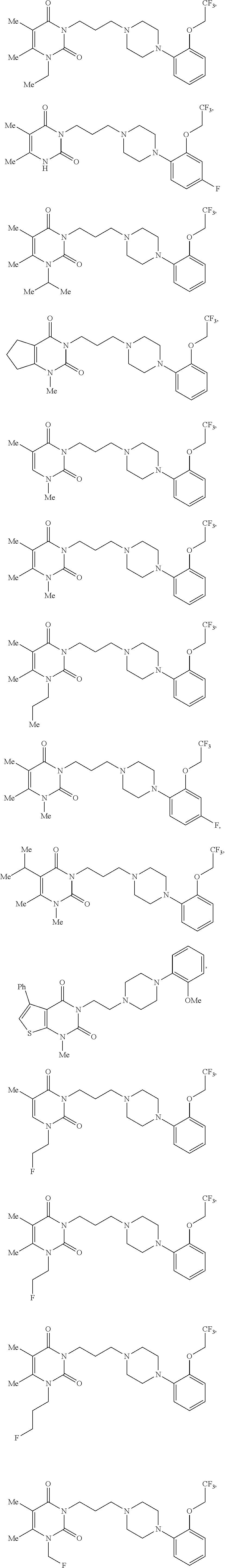

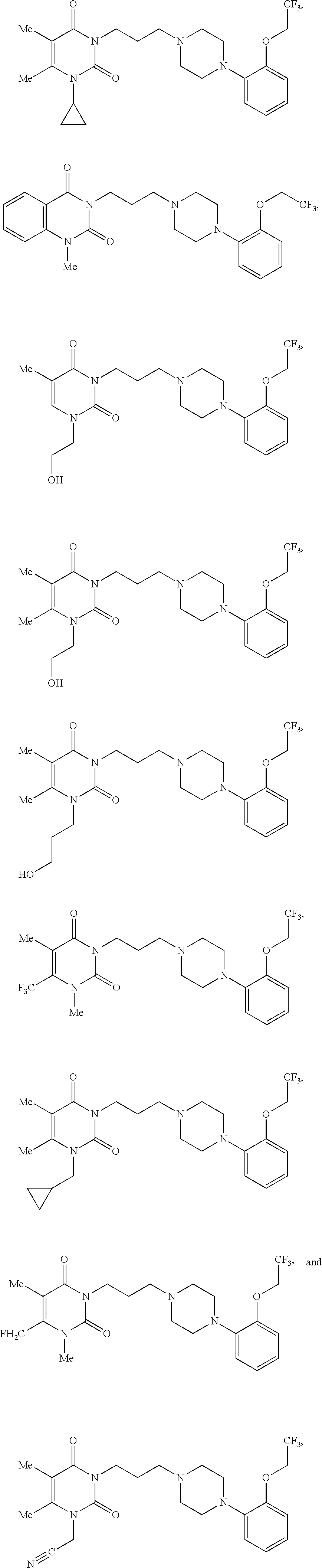

or pharmaceutically acceptable salts thereof.

Embodiment 14. The method of Embodiments 1-13 or any other Embodiment, wherein the radiolabeled compound comprises a carbon-11 (¹¹C), a carbon-14 (¹⁴C), a tritium (3H), or fluorine-18 (¹⁸F) radiolabel.

Embodiment 15. The method of Embodiments 1-14 or any other Embodiment, wherein a carbon of the alkyl, haloalkyl, hydroxyl alkyl, cycloalkyl, or alkyl-cycloalkyl group of R³ is ¹¹C.

Embodiment 16. The method of Embodiments 1-15 or any other Embodiment, wherein a halide of the haloalkyl group of R⁴ is ¹⁸F.

Embodiment 17. The method of Embodiments 1-16 or any other Embodiment, wherein a halide of the haloalkyl group of R² or a halide of the linear or branched haloalkyl group of R³ is ¹⁸F.

Embodiment 18. The method of Embodiments 1-17 or any other Embodiment, wherein the radio-imaging comprises positron emission tomography (PET) scanning.

Embodiment 19. A method for in vivo imaging of a tissue expressing an α1-AR receptor in a subject, the method comprising: administering an effective amount of a radiolabeled compound of Formula (I), or a pharmaceutical salt thereof, to the subject, wherein Formula (I) is

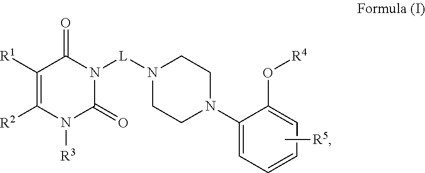

wherein: R¹ is a C₁-C₆ alkyl or a C₁-C₆ haloalkyl; R² is H, a C₁-C₆ alkyl, or a C₁-C₆ haloalkyl; or R¹ and R² together form a 5- or 6-membered cycloalkyl ring, a substituted or unsubstituted thiophenyl, or a substituted or unsubstituted phenyl; R³ is H, a C₁-C₆ alkyl, a C₁-C₆ haloalkyl, a C₁-C₆ hydroxyl alkyl, a C₃-C₆ cycloalkyl, a C₁-C₆ alkylnitrile, or an C₁-C₆ alkylcycloalkyl; R⁴ is C₁-C₆ alkyl or C₁-C₆ haloalkyl; R⁵ is H or halide; and L is a 2 to 10 carbon linking group; provided that: when R¹ is Me, R² is Me, R³ is H, R⁴ is —OCH₂CF₃, and L is —CH₂—CH₂—CH₂—, R⁵ is not H; and when R¹ is Me, R² is H, R³ is H, R⁴ is —OCH₂CF₃, and L is —CH₂—CH₂—CH₂—, R⁵ is not H; and radio-imaging at least a portion of the subject, wherein a position of a radio signal of a resultant radiograph corresponds to a site of the α1-AR receptor in the subject.

Embodiment 20. A radiolabeled compound of Formula (I)

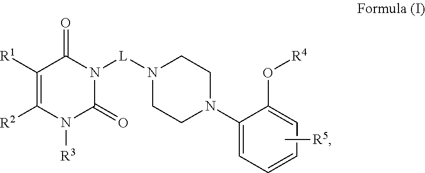

or a pharmaceutically acceptable salt thereof, wherein: R¹ is a C₁-C₆ alkyl or a C₁-C₆ haloalkyl; R² is H, a C₁-C₆ alkyl, or a C₁-C₆ haloalkyl; or R¹ and R² together form a 5- or 6-membered cycloalkyl ring, a substituted or unsubstituted thiophenyl, or a substituted or unsubstituted phenyl; R³ is H, a C₁-C₆ alkyl, a C₁-C₆ haloalkyl, a C₁-C₆ hydroxyl alkyl, a C₃-C₆ cycloalkyl, a C₁-C₆ alkylnitrile, or an C₁-C₆ alkylcycloalkyl; R⁴ is C₁-C₆ alkyl or C₁-C₆ haloalkyl; R⁵ is H or halide; and L is a 2 to 10 carbon linking group; provided that: when R¹ is Me, R² is Me, R³ is H, R⁴ is —OCH₂CF₃, and L is —CH₂—CH₂—CH₂—, R⁵ is not H; and when R¹ is Me, R² is H, R³ is H, R⁴ is —OCH₂CF₃, and L is —CH₂—CH₂—CH₂—, R⁵ is not H.

Embodiment 21. The radiolabeled compound of Embodiment 20 or any other Embodiment, wherein R¹ is methyl or isopropyl.

Embodiment 22. The radiolabeled compound of Embodiment 20 or Embodiment 21 or any other Embodiment, wherein R² is H or Me.

Embodiment 23. The radiolabeled compound of Embodiments 20-22 or any other Embodiment, wherein R² is CH₂F, CHF₂, or CF₃.

Embodiment 24. The radiolabeled compound of Embodiments 20-23 or any other Embodiment, wherein R¹ and R² together form

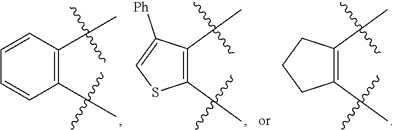 [SMILES: *c1ccccc1* ; *c1scc(-c2ccccc2)c1* ; *C1=C(*)CCC1]

Embodiment 25. The radiolabeled compound of Embodiments 20-24 or any other Embodiment, wherein R³ is H, CH₂CN, methyl, ethyl, propyl, or isopropyl.

Embodiment 26. The radiolabeled compound of Embodiments 20-25 or any other Embodiment, wherein R³ is fluoromethyl, fluoroethyl, or fluoropropyl.

Embodiment 27. The radiolabeled compound of Embodiments 20-26 or any other Embodiment, wherein R³ is hydroxyethyl or hydroxypropyl.

Embodiment 28. The radiolabeled compound of Embodiments 20-27 or any other Embodiment, wherein R³ is cyclopropyl or methyl cyclopropyl.

Embodiment 29. The radiolabeled compound of Embodiments 20-28 or any other Embodiment, wherein R⁴ is Me or CH₂CF₃.

Embodiment 30. The radiolabeled compound of Embodiments 20-29 or any other Embodiment, wherein R⁵ is F.

Embodiment 31. The radiolabeled compound of Embodiments 20-30 or any other Embodiment, wherein L is —(CH₂)ₙ, wherein n=2 or 3.

Embodiment 32. The radiolabeled compound of Embodiments 20-31 or any other Embodiment, wherein the radiolabeled compound of Formula (I) is selected from the group consisting of:

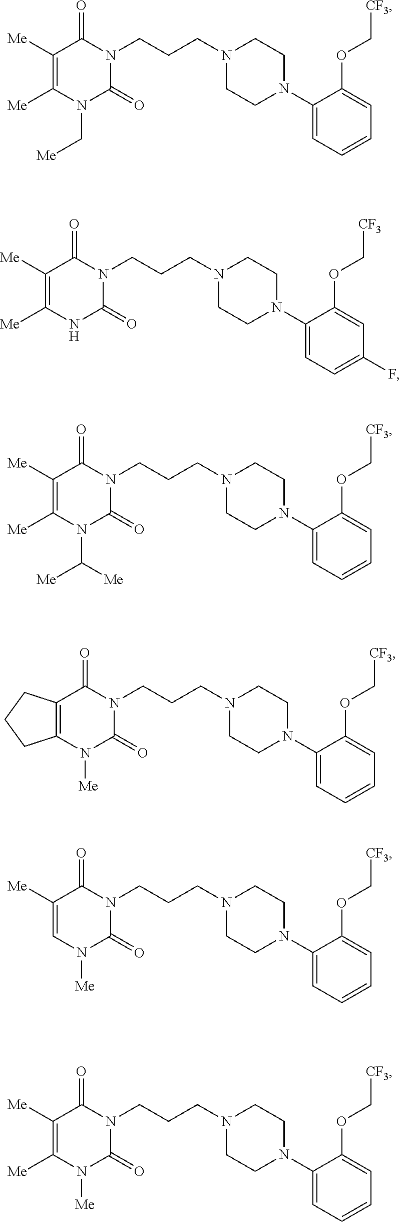 [SMILES: CCn1c(C)c(C)c(=O)n(CCCN2CCN(c3ccccc3OCC(F)(F)F)CC2)c1=O ; Cc1[nH]c(=O)n(CCCN2CCN(c3ccc(F)cc3OCC(F)(F)F)CC2)c(=O)c1C ; Cc1c(C)n(C(C)C)c(=O)n(CCCN2CCN(c3ccccc3OCC(F)(F)F)CC2)c1=O ; Cn1c2c(c(=O)n(CCCN3CCN(c4ccccc4OCC(F)(F)F)CC3)c1=O)CCC2 ; Cc1cn(C)c(=O)n(CCCN2CCN(c3ccccc3OCC(F)(F)F)CC2)c1=O ; Cc1c(C)n(C)c(=O)n(CCCN2CCN(c3ccccc3OCC(F)(F)F)CC2)c1=O]

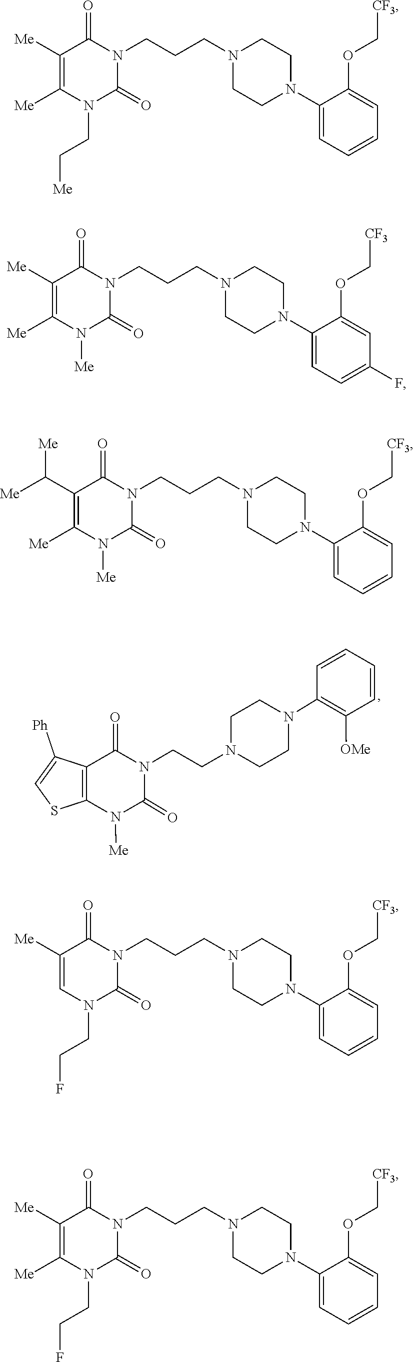 [SMILES: CCCn1c(C)c(C)c(=O)n(CCCN2CCN(c3ccccc3OCC(F)(F)F)CC2)c1=O ; Cc1c(C)n(C)c(=O)n(CCCN2CCN(c3ccc(F)cc3OCC(F)(F)F)CC2)c1=O ; Cc1c(C(C)C)c(=O)n(CCCN2CCN(c3ccccc3OCC(F)(F)F)CC2)c(=O)n1C ; COc1ccccc1N1CCN(CCn2c(=O)c3c(-c4ccccc4)csc3n(C)c2=O)CC1 ; Cc1cn(CCF)c(=O)n(CCCN2CCN(c3ccccc3OCC(F)(F)F)CC2)c1=O ; Cc1c(C)n(CCF)c(=O)n(CCCN2CCN(c3ccccc3OCC(F)(F)F)CC2)c1=O]

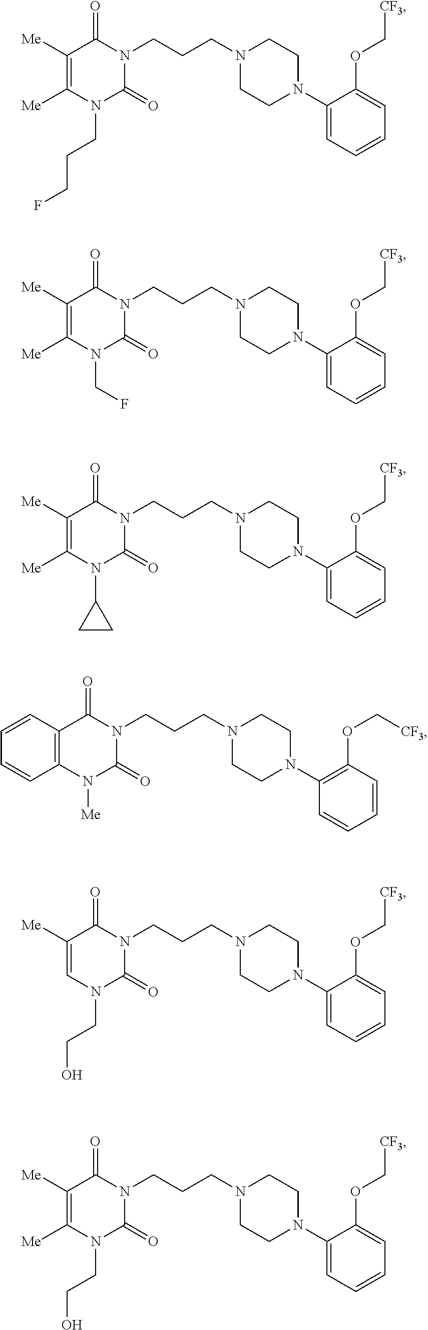 [SMILES: Cc1c(C)n(CCCF)c(=O)n(CCCN2CCN(c3ccccc3OCC(F)(F)F)CC2)c1=O ; Cc1c(C)n(CF)c(=O)n(CCCN2CCN(c3ccccc3OCC(F)(F)F)CC2)c1=O ; Cc1c(C)n(C2CC2)c(=O)n(CCCN2CCN(c3ccccc3OCC(F)(F)F)CC2)c1=O ; Cn1c(=O)n(CCCN2CCN(c3ccccc3OCC(F)(F)F)CC2)c(=O)c2ccccc21 ; Cc1cn(CCO)c(=O)n(CCCN2CCN(c3ccccc3OCC(F)(F)F)CC2)c1=O ; Cc1c(C)n(CCO)c(=O)n(CCCN2CCN(c3ccccc3OCC(F)(F)F)CC2)c1=O]

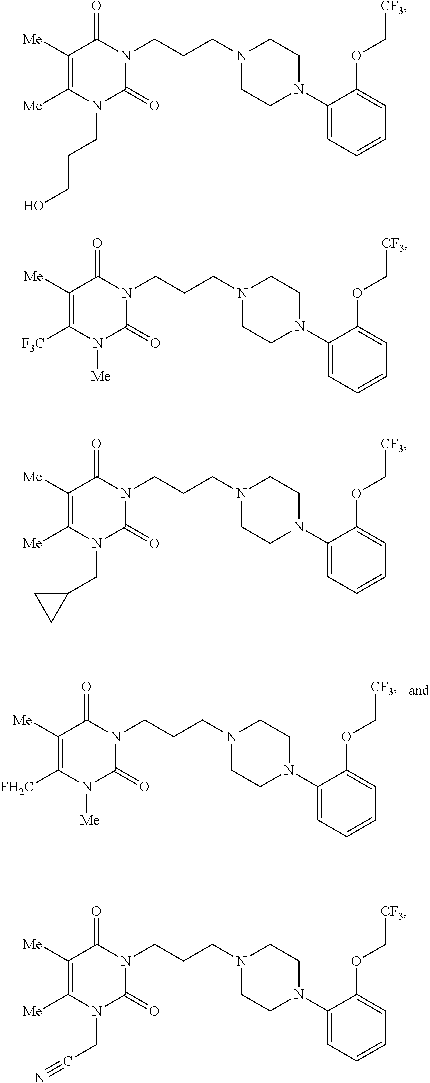

or pharmaceutically acceptable salts thereof.

Embodiment 33. The radiolabeled compound of Embodiments 20-32 or any other Embodiment, wherein the radiolabeled compound comprises a carbon-11 (¹¹C), a carbon-14 (¹⁴C), a tritium (³H), or fluorine-18 (¹⁸F) radiolabel.

Embodiment 34. The radiolabeled compound of Embodiments 20-33 or any other Embodiment, wherein a carbon of the alkyl, haloalkyl, hydroxyl alkyl, cycloalkyl, or alkyl-cycloalkyl group of R³ is ¹¹C.

Embodiment 35. The radiolabeled compound of Embodiments 20-34 or any other Embodiment, wherein a halide of the haloalkyl group of R⁴ is ¹⁸F.

Embodiment 36. The radiolabeled compound of Embodiments 20-35 or any other Embodiment, wherein a halide of the haloalkyl group of R² or a halide of the linear or branched haloalkyl group of R³ is ¹⁸F.

Embodiment 37. The radiolabeled compound of Embodiments 20-36 or any other Embodiment, wherein the present disclosure provides a pharmaceutical composition comprising any of the radiolabeled compounds described herein and a pharmaceutically acceptable carrier.

Embodiment 38. A method for synthesizing any of the radiolabeled compounds described herein, the method comprising Reaction I:

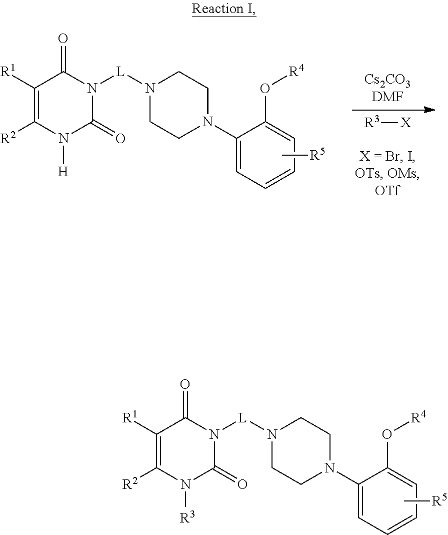

wherein R³ is a substituent selected from the group consisting of: a carbon-11 (¹¹C), carbon-14 (¹⁴C), or tritium (³H) labeled C₁-C₆ alkyl; a carbon-11 (¹¹C), carbon-14 (¹⁴C), tritium (3H), or fluorine-18 (¹⁸F) labeled C₁-C₆ haloalkyl; a carbon-11 (¹¹C), carbon-14 (¹⁴C), or tritium (³H) labeled C₁-C₆ hydroxyl alkyl; a carbon-11 (¹¹C), carbon-14 (¹⁴C), or tritium (³H) labeled C₃-C₆ cycloalkyl; a carbon-11 (¹¹C), carbon-14 (¹⁴C), or tritium (³H) labeled C₁-C₆ alkylnitrile; and a carbon-11 (¹¹C), carbon-14 (¹⁴C), or tritium (³H) labeled C₁-C₆ alkylcycloalkyl.

Embodiment 39. A method for predicting responsiveness of an al-AR receptor a subject to an α1-AR-targeted drug, for assessment of a treatment protocol for a medical condition, the method comprising: administering an effective amount of any of the radiolabeled compounds of the present disclosure, or a pharmaceutically acceptable salt thereof, to the subject; and radio-imaging at least a portion of a site of an α1-AR receptor in the subject, wherein a resultant radiograph comprises a radioactivity detection, a nuclear imaging scan, or another imaging, and corresponds to a predicted positive responsiveness of the subject to the radiolabeled compound, or a pharmaceutically acceptable salt thereof.

Embodiment 40. The method of any of Embodiments 1-19 or 38-39 or any other Embodiment, wherein the medical condition is selected from the group consisting of: post-traumatic stress disorder (PTSD), post-traumatic headaches, behavioral dysfunction (agitation and/or aggression) in Alzheimer's disease, alcohol use disorder, urinary dysfunction, autonomic dysreflexia in patients with spinal cord injuries, congestive heart failure (CHF), and any combination thereof.

Embodiment 41. The method of any of Embodiments 1-19 or 38-40 or any other Embodiment, wherein the tissue is selected from the group consisting of: central nervous system tissue, cardiovascular system tissue, heart tissue, bladder tissue, prostate tissue, muscle tissue, adipose tissue, brown adipose tissue, and any combination thereof.

Embodiment 42. The method of any of Embodiments 1-19 or 38-41 or any other Embodiment, wherein the α1-AR-targeted drug is a α1A-AR-targeted drug, an α1B-AR-targeted drug, an α1D-AR-targeted drug, or any combination thereof.

Embodiment 43. The method of any of Embodiments 1-19 or 38-42 or any other Embodiment, wherein the α1-AR receptor is a α1A-AR receptor, an α1B-AR receptor, or an α1D-AR receptor.

While illustrative embodiments have been illustrated and described, it will be appreciated that various changes can be made therein without departing from the spirit and scope of the invention.

1. A method for assessment of a treatment protocol for a subject with a medical condition that is treatable by targeting an α1-AR receptor, the method comprising:
screening the subject for prediction of responsiveness of the subject to an α1-AR-targeted drug, wherein the screening comprises:
administering an effective amount of a radiolabeled compound of Formula (I), or a pharmaceutical salt thereof, to the subject, wherein Formula (I) is 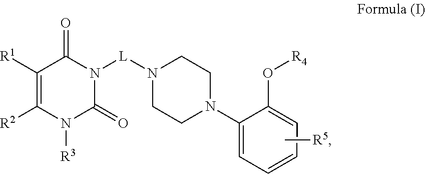
wherein:
R¹ is a C₁-C₆ alkyl or a C₁-C₆ haloalkyl;
R² is H, a C₁-C₆ alkyl, or a C₁-C₆ haloalkyl; or
R¹ and R² together form a 5- or 6-membered cycloalkyl ring, a substituted or unsubstituted thiophenyl, or a substituted or unsubstituted phenyl;
R³ is H, a C₁-C₆ alkyl, a C₁-C₆ haloalkyl, a C₁-C₆ hydroxyl alkyl, a C₃-C₆ cycloalkyl, a C₁-C₆ alkylnitrile, or an C₁-C₆ alkylcycloalkyl;
R⁴ is C₁-C₆ alkyl or C₁-C₆ haloalkyl;
R⁵ is H or halide; and
L is a 2 to 10 carbon linking group;
provided that:
when R¹ is Me, R² is Me, R³ is H, R⁴ is —OCH₂CF₃, and L is —CH₂—CH₂—CH₂—, R⁵ is not H; and
when R¹ is Me, R² is H, R³ is H, R⁴ is —OCH₂CF₃, and L is —CH₂—CH₂—CH₂—, R⁵ is not H; and
radio-imaging at least a portion of a site of an α1-AR receptor in a subject, wherein a resultant radiograph corresponds to a predicted responsiveness of the subject to the radiolabeled compound, or a pharmaceutically acceptable salt thereof, and
administering an effective amount of the α1-AR-targeted drug to the subject based on the predicted responsiveness, wherein the α1-AR-targeted drug is a non-radiolabeled derivative of the radiolabeled compound of Formula (I).

2. The method of claim 1, wherein R¹ is methyl or isopropyl.

3. The method of claim 1, wherein R² is H or Me.

4. The method of claim 1, wherein R² is CH₂F, CHF₂, or CF₃.

5. The method of claim 1, wherein R¹ and R² together form

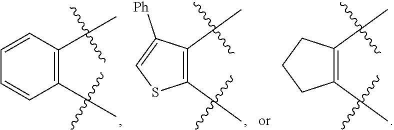 [SMILES: *c1ccccc1* ; *c1scc(-c2ccccc2)c1* ; *C1=C(*)CCC1]

6. The method of claim 1, wherein R³ is H, CH₂CN, methyl, ethyl, propyl, or isopropyl.

7. The method of claim 1, wherein R³ is fluoromethyl, fluoroethyl, or fluoropropyl.

8. The method of claim 1, wherein R³ is hydroxyethyl or hydroxypropyl.

9. The method of claim 1, wherein R³ is cyclopropyl or methyl cyclopropyl.

10. The method of claim 1, wherein R⁴ is Me or CH₂CF₃.

11. The method of claim 1, wherein R⁵ is F.

12. The method of claim 1, wherein L is —(CH₂)ₙ, wherein n=2 or 3.

13. The method of claim 1, wherein the radiolabeled compound of Formula (I) is selected from the group consisting of: 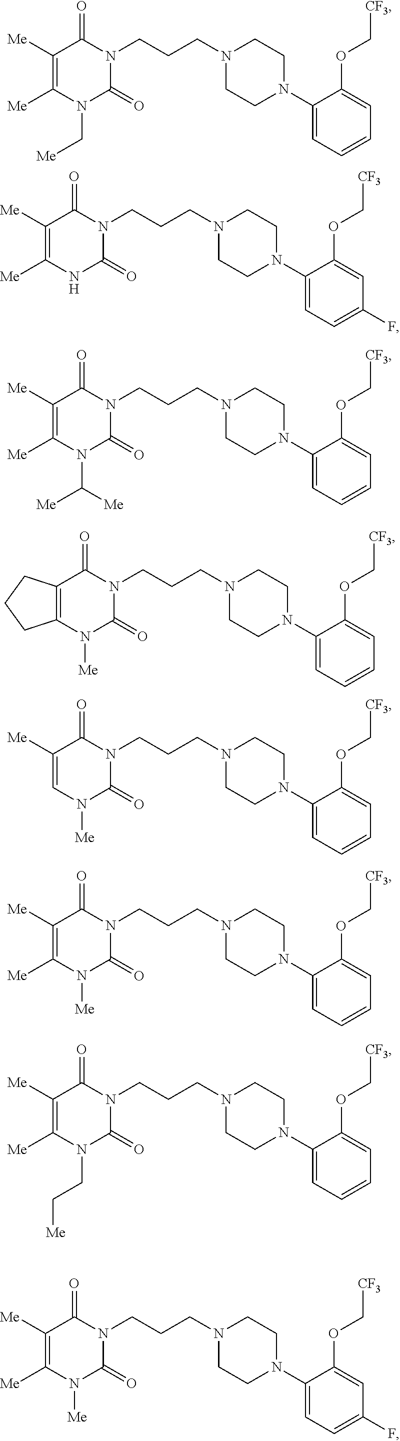 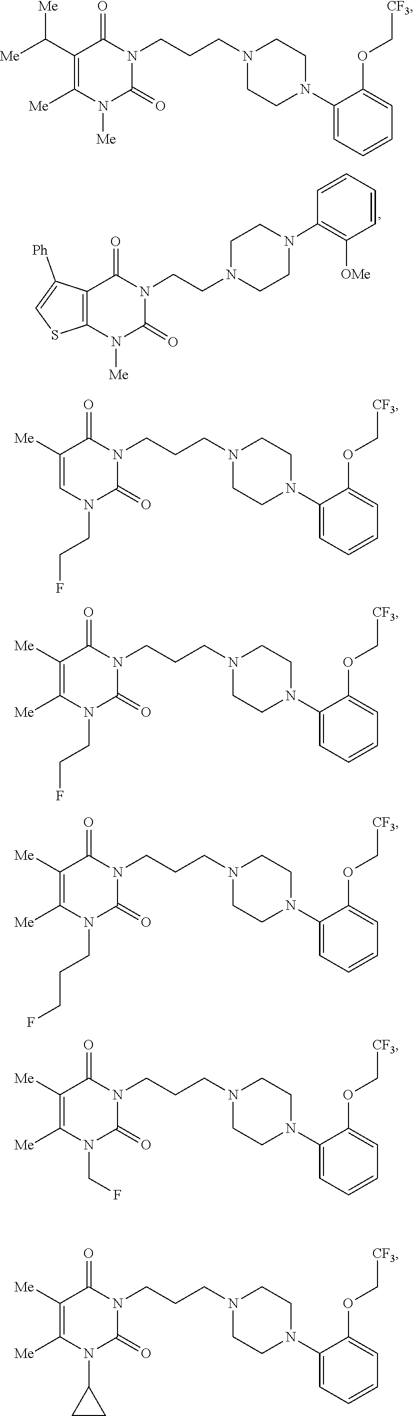 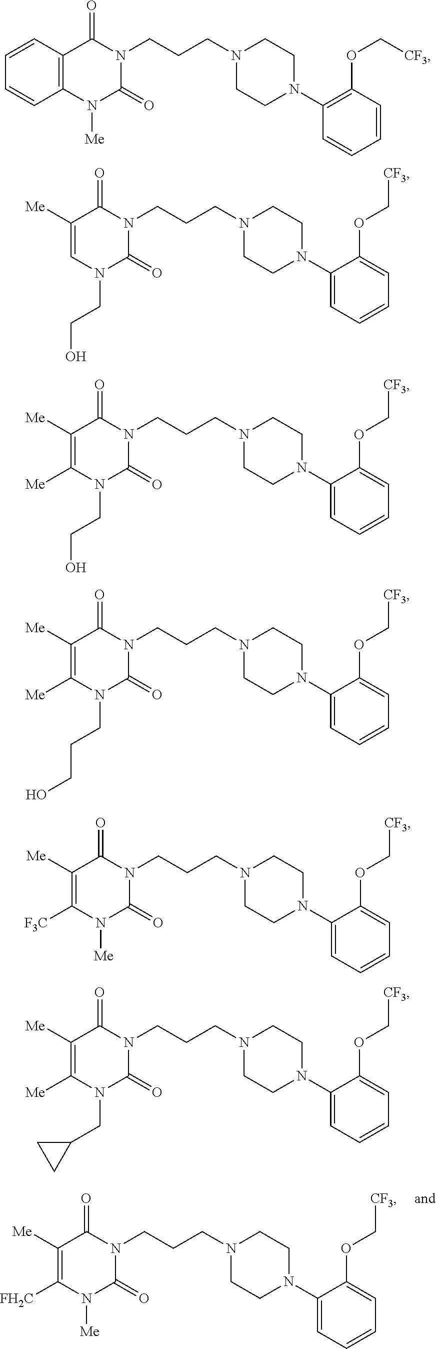 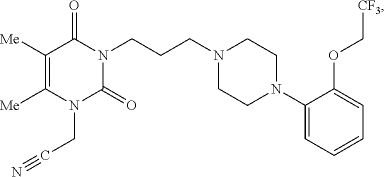 [SMILES: Cc1c(C)n(CC#N)c(=O)n(CCCN2CCN(c3ccccc3OCC(F)(F)F)CC2)c1=O]
or pharmaceutically acceptable salts thereof.

14. The method of claim 1, wherein the radiolabeled compound comprises a carbon-11 (¹¹C), a carbon-14 (¹⁴C), a tritium (³H), or fluorine-18 (¹⁸F) radiolabel.

15. The method of claim 14, wherein a carbon of the alkyl, haloalkyl, hydroxyl alkyl, cycloalkyl, or alkyl-cycloalkyl group of R³ is ¹¹C.

16. The method of claim 14, wherein a halide of the haloalkyl group of R⁴ is ¹⁸F.

17. The method of claim 14, wherein a halide of the haloalkyl group of R² or a halide of the linear or branched haloalkyl group of R³ is ¹⁸F.

18. The method of claim 1, wherein the radio-imaging comprises positron emission tomography (PET) scanning.

19. A method for in vivo imaging of a tissue expressing an α1-AR receptor in a subject, the method comprising:
administering an effective amount of a radiolabeled compound of Formula (I), or a pharmaceutical salt thereof, to the subject, wherein Formula (I) is 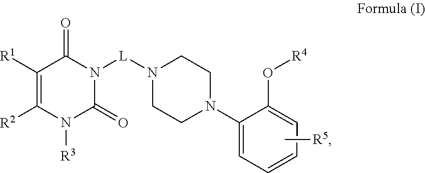
wherein:
R¹ is a C₁-C₆ alkyl or a C₁-C₆ haloalkyl;
R² is H, a C₁-C₆ alkyl, or a C₁-C₆ haloalkyl; or
R¹ and R² together form a 5- or 6-membered cycloalkyl ring, a substituted or unsubstituted thiophenyl, or a substituted or unsubstituted phenyl;
R³ is H, a C₁-C₆ alkyl, a C₁-C₆ haloalkyl, a C₁-C₆ hydroxyl alkyl, a C₃-C₆ cycloalkyl, a C₁-C₆ alkylnitrile, or an C₁-C₆ alkylcycloalkyl;
R⁴ is C₁-C₆ alkyl or C₁-C₆ haloalkyl;
R⁵ is H or halide; and
L is a 2 to 10 carbon linking group;
provided that:
when R¹ is Me, R² is Me, R³ is H, R⁴ is —OCH₂CF₃, and L is —CH₂—CH₂—CH₂—, R⁵ is not H; and
when R¹ is Me, R² is H, R³ is H, R⁴ is —OCH₂CF₃, and L is —CH₂—CH₂—CH₂—, R⁵ is not H; and
radio-imaging at least a portion of the subject, wherein a position of a radio signal of a resultant radiograph corresponds to a site of the α1-AR receptor in the subject.

20. A radiolabeled compound of Formula (I) 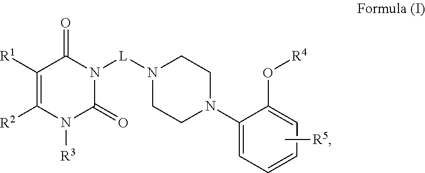
or a pharmaceutically acceptable salt thereof,
wherein:
R¹ is a C₁-C₆ alkyl or a C₁-C₆ haloalkyl;
R² is H, a C₁-C₆ alkyl, or a C₁-C₆ haloalkyl; or
R¹ and R² together form a 5- or 6-membered cycloalkyl ring, a substituted or unsubstituted thiophenyl, or a substituted or unsubstituted phenyl;
R³ is H, a C₁-C₆ alkyl, a C₁-C₆ haloalkyl, a C₁-C₆ hydroxyl alkyl, a C₃-C₆ cycloalkyl, a C₁-C₆ alkylnitrile, or an C₁-C₆ alkylcycloalkyl;
R⁴ is C₁-C₆ alkyl or C₁-C₆ haloalkyl;
R⁵ is H or halide; and
L is a 2 to 10 carbon linking group;
provided that:
when R¹ is Me, R² is Me, R³ is H, R⁴ is —OCH₂CF₃, and L is —CH₂—CH₂—CH₂—, R⁵ is not H; and
when R¹ is Me, R² is H, R³ is H, R⁴ is —OCH₂CF₃, and L is —CH₂—CH₂—CH₂—, R⁵ is not H.
